(19) United States
(12) Patent Application Publication
(10) Pub. No.: US 2026/0256726 A1
(43) Pub. Date: Sep. 3, 2026

(54) METHODS AND COMPOSITIONS RELATING TO TREATMENT AND PREVENTION OF FATTY ACID DEFICIENCIES

(71) Applicant: THE CHILDREN'S MEDICAL CENTER CORPORATION, Boston, MA (US)

(72) Inventors: Mark PUDER, Medfield, MA (US); Scott FLIGOR, Boston, MA (US); Kathleen GURA, Boston, MA (US)

(73) Assignee: THE CHILDREN'S MEDICAL CENTER CORPORATION, Boston, MA (US)

(21) Appl. No.: 18/717,477

(22) PCT Filed: Dec. 7, 2022
(86) PCT No.: PCT/US2022/052076

§ 371 Date: Jun. 7, 2024

Related U.S. Application Data
(60) Provisional application No. 63/287,564, filed on Dec. 9, 2021.

(51) Int. Cl. A61K 31/202; A61K 35/60; A61K 36/02; A61P 3/02

(52) U.S. Cl. CPC A61K 31/202; A61K 35/60; A61K 36/02; A61P 3/02

(57) ABSTRACT

Described herein are compositions and methods relating to lipid formulations (e.g, lipid emulsions) comprising a) Arachidonic acid (ARA); and b) Docosahexaenoic acid (DHA) and/or Eicosapentaenoic acid (EPA).

### CROSS-REFERENCE TO RELATED APPLICATIONS

This application claims benefit under 35 U.S.C. § 119(e) of U.S. Provisional Application No. 63/287,564 filed Dec. 9, 2021, the contents of which are incorporated herein by reference in their entirety.

### TECHNICAL FIELD

The technology described herein relates to lipid emulsions for administration to patients, e.g., orally or parenterally.

### BACKGROUND

Neonatal patients, particularly those in intensive care units (ICUs), can require parenteral nutrition. However, existing lipid emulsions are formulated for adults and therefore have issues with providing a sufficient amount of nutrients to newborns (particularly preterm infants) due to total volume, lipid content, toxicology concerns, etc. For example, fluid administration in neonates is extremely challenging due to fluid shifts, physiological changes in kidney function, and high insensible sensible losses. Standard emulsions can accelerate or hasten complications of prematurity such as retinopathy, bronchopulmonary dysplasia, liver injury, and cholestasis.

### SUMMARY

Described herein are new emulsion formulations that avoid or reduce total volume and toxicology side effects when administered to newborns. The present emulsions therefore permit method of providing necessary fatty acids/nutrients to newborn patients, particularly preterm infants.

In one aspect of any of the embodiments, described herein is a composition comprising: a) one or more omega-6 fatty acids comprising Arachidonic acid (ARA); and b) one or more omega-3 fatty acids comprising Docosahexaenoic acid (DHA) and/or Eicosapentaenoic acid (EPA). In one aspect of any of the embodiments, described herein is a composition comprising: a) Arachidonic acid (ARA); and b) Docosahexaenoic acid (DHA) and/or Eicosapentaenoic acid (EPA). In some embodiments of any of the aspects the composition comprises DHA. In some embodiments of any of the aspects, the composition comprises EPA. In some embodiments of any of the aspects, the composition comprises EPA and DHA.

In some embodiments of any of the aspects, the ratio of a) ARA to b) DHA and/or EPA is from 3:2 to 3:5 by weight. In some embodiments of any of the aspects, the ratio of a) ARA to b) DHA and/or EPA is from 1:2 to 3:5 by weight. In some embodiments of any of the aspects, the ratio of a) ARA to b) DHA and/or EPA is from 1:2 to 1:5 by weight. In some embodiments of any of the aspects, the ratio of a) ARA to b) DHA and/or EPA is from 2:1 to 1:4 by weight. In some embodiments of any of the aspects, the ratio of a) ARA to b) DHA and/or EPA is from 1:1 to 1:2 by weight.

In some embodiments of any of the aspects, the composition comprises further omega-6 fatty acids and/or omega-3 fatty acids. In some embodiments of any of the aspects, the ARA and DHA and/or EPA comprise at least 60% of the total omega-3 fatty acids and omega-6 fatty acids. In some embodiments of any of the aspects, the ARA and DHA and/or EPA comprise at least 70% of the total omega-3 fatty acids and omega-6 fatty acids. In some embodiments of any of the aspects, the ARA and DHA and/or EPA comprise at least 80% of the total omega-3 fatty acids and omega-6 fatty acids. In some embodiments of any of the aspects, the ARA and DHA and/or EPA comprise at least 90% of the total omega-3 fatty acids and omega-6 fatty acids.

In some embodiments of any of the aspects, the ARA comprises at least 20% of the total omega-6 fatty acids. In some embodiments of any of the aspects, the ARA comprises at least 30% of the total omega-6 fatty acids. In some embodiments of any of the aspects, the ARA comprises at least 40% of the total omega-6 fatty acids. In some embodiments of any of the aspects, the DHA and/or EPA comprises at least 20% of the total omega-3 fatty acids. In some embodiments of any of the aspects, the DHA and/or EPA comprises at least 30% of the total omega-3 fatty acids. In some embodiments of any of the aspects, the DHA and/or EPA comprises at least 40% of the total omega-3 fatty acids. In some embodiments of any of the aspects, the ratio of omega-6 fatty acids to omega-3 fatty acids is from 2:1 to 1:4 by weight. In some embodiments of any of the aspects, the ratio of omega-6 fatty acids to omega-3 fatty acids is from 1:1 to 1:2 by weight.

In some embodiments of any of the aspects, the composition and the components thereof are not distilled or re-esterified. In some embodiments of any of the aspects, the total triglyceride+diglyceride content of the composition comprises no more than 10% diglyceride. In some embodiments of any of the aspects, the composition comprises no diglyceride. In some embodiments of any of the aspects, the DHA and/or EPA is not distilled or re-esterified. In some embodiments of any of the aspects, the total triglyceride+diglyceride content of the DHA and/or EPA comprises no more than 10% diglyceride. In some embodiments of any of the aspects, the DHA and/or EPA comprises no diglyceride. In some embodiments of any of the aspects, the ARA is not distilled or re-esterified. In some embodiments of any of the aspects, the total triglyceride+diglyceride content of the ARA comprises no more than 10% diglyceride. In some embodiments of any of the aspects, the ARA comprises no diglyceride.

In some embodiments of any of the aspects, the composition comprises no more than 5% (w/w) sterols. In some embodiments of any of the aspects, the composition comprises no more than 2% (w/w) sterols. In some embodiments of any of the aspects, the composition comprises no more than 1.5% (w/w) sterols. In some embodiments of any of the aspects, the composition comprises no more than 120 mg/L phytosterols. In some embodiments of any of the aspects, the composition comprises no more than 70 mg/L phytosterols. In some embodiments of any of the aspects, the composition comprises no more than 35 mg/L phytosterols. In some embodiments of any of the aspects, the composition comprises no more than 4% (w/w) stigmasterol. In some embodiments of any of the aspects, the composition comprises no more than 10 mg/L stigmasterol.

In some embodiments of any of the aspects, the composition comprises, or the ARA is provided in the form of a plant or fungal oil. In some embodiments of any of the aspects, the plant or fungal oil is not distilled or re-esterified. In some embodiments of any of the aspects, the total triglyceride+diglyceride content of the plant or fungal oil comprises no more than 10% diglyceride. In some embodiments of any of the aspects, the plant or fungal oil comprises no diglyceride. In some embodiments of any of the aspects, the plant or fungal oil comprises no more than 5% (w/w) sterols, no more than 70 mg/L phytosterols, no more than 4% (w/w) stigmasterol, and/or no more than 10 mg/L stigmasterol. In some embodiments of any of the aspects, the plant oil is soybean oil or olive oil, or the fungal oil is Mortierella alpina oil.

In some embodiments of any of the aspects, the composition comprises a fish oil, and/or the DHA and/or EPA is provided in the form of fish oil. In some embodiments of any of the aspects, the fish oil is not distilled or re-esterified. In some embodiments of any of the aspects, the total triglyceride+diglyceride content of the fish oil comprises no more than 10% diglyceride. In some embodiments of any of the aspects, the fish oil comprises no diglyceride. In some embodiments of any of the aspects, the fish oil comprises no more than 5% (w/w) sterols, no more than 70 mg/L phytosterols, no more than 4% (w/w) stigmasterol, and/or no more than 10 mg/L stigmasterol.

In some embodiments of any of the aspects, the composition comprises an algal oil, and/or the DHA and/or EPA is provided in the form of algal oil. In some embodiments of any of the aspects, the algal oil is oil from Crypthecodinium cohnii. In some embodiments of any of the aspects, the algal oil is not distilled or re-esterified. In some embodiments of any of the aspects, the total triglyceride+diglyceride content of the algal oil comprises no more than 10% diglyceride. In some embodiments of any of the aspects, the algal oil comprises no diglyceride. In some embodiments of any of the aspects, the algal oil comprises no more than 5% (w/w) sterols, no more than 70 mg/L phytosterols, no more than 4% (w/w) stigmasterol, and/or no more than 10 mg/L stigmasterol.

In some embodiments of any of the aspects, the composition is a lipid emulsion. In some embodiments of any of the aspects, the emulsion is at least 10% oil in water. In some embodiments of any of the aspects, the emulsion is at least 20% oil in water. In some embodiments of any of the aspects, the emulsion is at least 30% oil in water. In some embodiments of any of the aspects, the emulsion is about 10% to about 50% oil in water. In some embodiments of any of the aspects, the emulsion is about 10% to about 40% oil in water. In some embodiments of any of the aspects, the emulsion is about 10% to about 30% oil in water. In some embodiments of any of the aspects, the emulsion is about 20% to about 40% oil in water.

In some embodiments of any of the aspects, the composition further comprises one or more of: medium chain triglycerides (MCTs); egg lecithin; sunflower seed oil; sunflower lecithin; an emulsifier obtained from sunflower seed; and krill oil. In some embodiments of any of the aspects, the composition is formulated to comprise a) ARA at a dosage of up to 300 mg/kg/day and/or b) DHA and/or EPA at a dosage of up to 500 mg/kg/day. In some embodiments of any of the aspects, the composition is formulated to comprise a) ARA at a dosage of up to 200 mg/kg/day and/or b) DHA and/or EPA at a dosage of up to 400 mg/kg/day. In some embodiments of any of the aspects, the composition is formulated to comprise a) ARA at a dosage of 20-60 mg/kg/day and/or b) DHA and/or EPA at a dosage of 40-100 mg/kg/day. In some embodiments of any of the aspects, the composition is formulated to comprise a) ARA at a dosage of 20-60 mg/kg/day and/or b) DHA and/or EPA at a dosage of 10 mg/kg/day-3 g/kg/day. In some embodiments of any of the aspects, the composition is formulated for parenteral or intravenous administration.

In one aspect of any of the embodiments, described herein is a method of providing nutrition to a subject and/or promoting neurodevelopment in a subject, the method comprising administering a composition described herein to the subject. In one aspect of any of the embodiments, described herein is a) Arachidonic acid (ARA); and b) Docosahexaenoic acid (DHA) and/or Eicosapentaenoic acid (EPA) for use in a method of providing nutrition to a subject and/or promoting neurodevelopment in a subject.

In some embodiments of any of the aspects, the composition is administered at a dose of no more than 5 g/kg/day. In some embodiments of any of the aspects, the composition is administered to provide a) ARA at a dosage of 20-60 mg/kg/day and/or b) DHA and/or EPA at a dosage of 40-100 mg/kg/day. In some embodiments of any of the aspects, the subject is an infant. In some embodiments of any of the aspects, the subject is a neonatal and/or premature infant. In some embodiments of any of the aspects, the neurodevelopment is neurodevelopment in the brain and/or eyes. In some embodiments of any of the aspects, the administration treats, prevents, or reduces the risk of one or more conditions selected from the group consisting of: retinopathy; bronchopulmonary dysplasia; and perinatal sepsis. In some embodiments of any of the aspects, the administration is parenteral and/or intravenous. In some embodiments of any of the aspects, the nutrition is parenteral nutrition or total parenteral nutrition. In some embodiments of any of the aspects, the administration is parenteral administration or total parenteral administration. In some embodiments of any of the aspects, the subject is in need of parenteral nutrition or total parenteral nutrition. In some embodiments of any of the aspects, the patient does not receive oral nutrition. In some embodiments of any of the aspects, the patient does not receive other parenteral formulations. In some embodiments of any of the aspects, the patient does not receive oral nutrition which is sufficient to maintain a nutritional balance. In some embodiments of any of the aspects, the patient does not receive other parenteral formulations which are sufficient to maintain a nutritional balance. In some embodiments of any of the aspects, the patient does not receive other nutritional sources and/or parenteral nutritional sources of fatty acids. In some embodiments of any of the aspects, the patient does not receive other nutritional sources and/or parenteral nutritional sources of essential fatty acids. In some embodiments of any of the aspects, the composition is administered as a monotherapy. In some embodiments of any of the aspects, the composition is administered as a monotherapy for nutritional needs.

In some embodiments of any of the aspects, the patient is a patient in need of treatment for a condition selected from the group consisting of: hepatic steatosis; intestinal failure; parenteral nutrition-associated liver disease (PNALD); sepsis; cystic fibrosis; sickle cell anemia; pancreatitis; inflammatory bowel disease; Crohn's disease; biliary atresia; primary sclerosis cholangitis; an inflammatory infection; an inflammatory condition; systemic inflammatory response syndrome (SIRS); hypertriglyceridemia; severe hypertriglyceridemia; severe hepatic steatosis; retinopathy of prematurity; acute tubular necrosis; IgA nephropathies; ischemia-reperfusion injury; traumatic brain injury; multi-system organ failure; respiratory distress syndrome; acute myocardial infarction; myocardial infarction; status anginosus; status asthmaticus; status epilepticus; status lacunaris; inflammatory bowel disease; regional enteritis; ulcerative colitis; severe or debilitating arthritis; arthritis; psoriasis; severe psoriasis; burns; third degree burns; pancreatitis; acute pancreatitis; intestinal failure associated liver disease (IFALD); parenteral nutrition associated cholestasis (PNAC); essential fatty acid deficiency (EFAD); parenteral nutrition dependency complicated by soy allergy; local anesthetic systemic toxicity; treatment of systemic poisoning; and need for a parenteral therapeutic drug vehicle or excipient.

### BRIEF DESCRIPTION OF THE DRAWINGS

FIGS. 1A-1H depict graphs of the fatty acid composition of plasma in 8 week old C57Bl/6J mice that received 4 weeks of ad lib fat-free high carbohydrate diet plus daily orogastric gavage of lipid emulsion (or normal rodent chow control with daily orogastric gavage of normal saline). Statistical comparisons made with one-way ANOVA and individual comparisons to chow control made with Dunnett's multiple comparisons test. *P<0.05 **P<0.01 ***P<0.001 ****P<0.0001.

FIGS. 2A-2H depict graphs of the fatty acid composition of liver in 8 week old C57Bl/6J mice that received 4 weeks of ad lib fat-free high carbohydrate diet plus daily orogastric gavage of lipid emulsion (or normal rodent chow control with daily orogastric gavage of normal saline). Statistical comparisons made with one-way ANOVA and individual comparisons to chow control made with Dunnett's multiple comparisons test. *P<0.05 **P<0.01 ***P<0.001 ****P<0.0001

FIGS. 3A-3H depict graphs of fatty acid composition of frontal cortex in 8 week old C57Bl/6J mice that received 4 weeks of ad lib fat-free high carbohydrate diet plus daily orogastric gavage of lipid emulsion (or normal rodent chow control with daily orogastric gavage of normal saline). Statistical comparisons made with one-way ANOVA and individual comparisons to chow control made with Dunnett's multiple comparisons test. *P<0.05 **P<0.01 ***P<0.001 ****P<0.0001

FIGS. 4A-4B depict graphs demonstrating 8 week old C57Bl/6J mice that received 4 weeks of ad lib fat-free high carbohydrate diet plus daily orogastric gavage of NLE A, NLE B, or NLE C do not demonstrate evidence of essential fatty acid deficiency, while mice receiving daily Intralipid orogastric gavage demonstrate elevated triene: tetraene ratios in both plasma and liver tissue concerning for essential fatty acid deficiency. Statistical comparisons made with one-way ANOVA and individual comparisons to chow control made with Dunnett's multiple comparisons test. *P<0.05 **P<0.01 ***P<0.001 ****P<0.0001

FIGS. 5A-5C demonstrate that mice receiving an ad lib fat-free high carbohydrate diet with every other day tail vein injection of saline develop biochemical liver injury marked by elevated alanine aminotransferase. Provision of every other day tail vein injection of lipid emulsion (or chow diet plus saline injection) prevents biochemical liver injury. Statistical comparison made with one way ANOVA test and Dunnett's multiple comparisons test against the saline group. Mean±SEM with individual values plotted. *P<0.05 **P<0.01 ***P<0.001

FIG. 6 demonstrates that mice receiving an ad lib fat-free high carbohydrate diet with every other day tail vein injection of saline or intralipid develop micro- and macrovesicular steatosis. Representative hematoxylin and eosin stained liver tissue is shown. Mice receiving the high carbohydrate diet with NLE B or NLE C have no steatosis, while some mice receiving NLE A demonstrate mild micro- and macro-vesicular steatosis. Mice receiving a chow diet (with saline tail vein injection) have normal histology with no steatosis.

FIG. 7 demonstrates that compared to mice receiving a high carbohydrate diet with saline tail vein injections for 19 days, mice in the NLE B, NLE C, Omegaven, and Chow groups demonstrated significantly lower lipidosis scores consistent with decreased steatosis. Mice in the Intralipid and NLE A groups did not have significantly lower lipidosis scores than the saline group. Statistical comparison made with Kruskal-Wallis test and Dunn's multiple comparisons test against the saline group. Mean±SEM with individual values plotted.*P<0.05 **P<0.01

FIGS. 8A-8F depict graphs of plasma fatty acid profiles and triene: tetraene ratio for mice receiving either chow control or a high carbohydrate diet with saline or lipid emulsion tail vein injections for 19 days. Statistical comparisons made with one-way ANOVA and individual comparisons to chow control made with Dunnett's multiple comparisons test. *P<0.05 **P<0.01 ***P<0.001 ****P<0.0001

### DETAILED DESCRIPTION

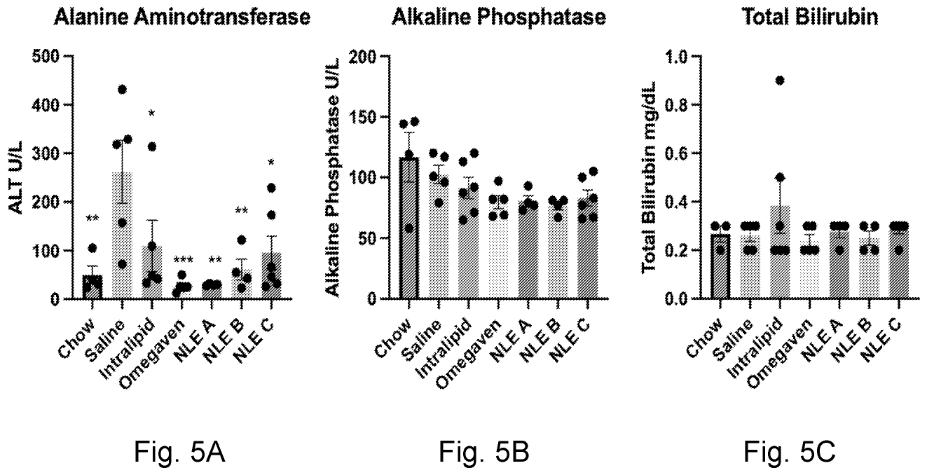

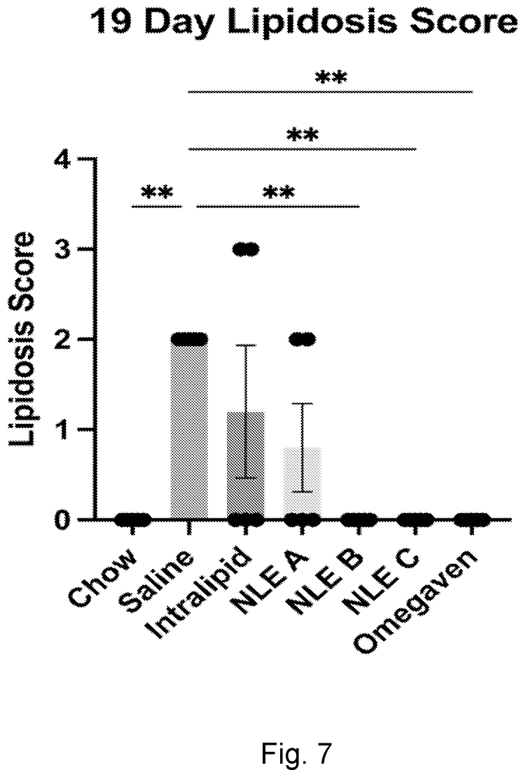

As described herein, fat-free and high-carbohydrate diets induce liver injury (e.g, as measured by alanine aminotransferase activity or steatosis). Supplementation with high omega-6 fatty acid soybean oil lipid emulsions (such as INTRALIPID) only partially reduce some aspects of the liver injury-patients receiving INTRALIPID, particularly as part of a TPN strategy, will experience liver injury at levels significantly above a normal diet and often at the same level as patients not receiving any fat (See, e.g., FIG. 5A and FIG. 7). Clinically, there is extensive evidence indicating that use of INTRALIPID leads to the development of intestinal failure-associated liver disease. Similar liver injury is seen with the use of lipid mixtures such as SMOFLIPID, which comprises soybean oil, medium chain triglycerides, olive oil, and fish oil. This liver injury can be avoided if the patient is instead provided with a high omega-3 fatty acid lipid emulsion (such as OMEGAVEN; See, e.g., FIG. 5A and FIG. 7). However, OMEGAVEN is a 10% fish oil in water emulsion, requiring relatively high total volumes of emulsion to be administered, often approaching or exceeding recommended volume limits for infants. Additionally, OMEGAVEN's total fat calories necessitate the administration of additional dextrose at doses higher than normally used to provide sufficient total calories.

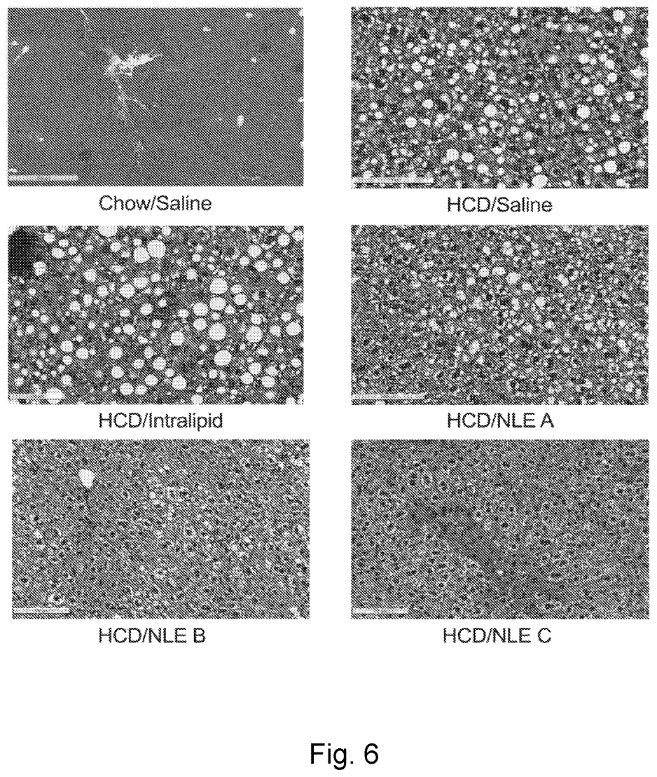

Surprisingly, the inventors have now found that this liver injury associated with administration of formulations such as INTRALIPID can be avoided by the addition of appropriate doeses of arachidonic acid (ARA). See FIGS. 5A, and 7. As shown in FIG. 7, the combination of a high DHA/EPA oil with a high ARA oil at 80% and 2.5%, respectively, (NLE A) actually increased steatosis relative to the high DHA/EPA oil alone. See FIG. 7. But when the proportion of the high ARA was increased (NLE B and NLE C), the steatosis increase was not observed. Histology confirmed this, finding that mice receiving NLE B or NLE C had no steatosis, while some mice receiving NLE A experienced mild micro- and macrovescular steatosis. See FIG. 6.

These new formulations also offer key advantages relative to fish oil emulsions such as OMEGAVEN. For example, the compositions described herein can be formulated as a greater than 10% emulsion, permitting administration of total volumes that are safer for infants. Additionally, the compositions described herein comprise sufficient fat calories to reduce the need for administration of dextrose and/or glucose (e.g., reduce the amount or frequency of dextrose administration) and provide substantially more ARA than OMEGAVEN.

Accordingly, described herein are compositions, e.g., emulsions, for administration, e.g, via Parenteral Nutrition (PN) or total parenteral nutition (TPN) that promote neurodevelopment in infants or newborns (e.g, premature infants or newborns). These compositions, e.g., emulsions, also provide a reduced risk of cholestatis, intestinal failure-associated liver disease (IFALD), retinopathy, and bronchopulmonary dysplasia. The compositions, e.g., emulsions, are particularly formulated for administration to infants, e.g. neonates and provide advantageous dosing volumes for such patients.

In one aspect of any of the embodiments, described herein is a composition comprising a) Arachidonic acid (ARA) and b) Docosahexaenoic acid (DHA) and/or Eicosapentaenoic acid (EPA). In some embodiments of any of the aspects, the composition is an emulsion. In some embodiments of any of the aspects, the composition comprises an emulsion. In some embodiments of any of the aspects, the composition comprises an emulsion of at least a) ARA and b) DHA and/or EPA. In some embodiments of any of the aspects, the composition comprises a mixture of a) an emulsion of at least ARA and b) an emulsion of at least DHA and/or EPA.

In one aspect of any of the embodiments, described herein is a composition comprising one or more omega-6 fatty acids comprising at least Arachidonic acid (ARA) and one or more omega-3 fatty acids comprising at least Docosahexaenoic acid (DHA) and/or EPA. In some embodiments of any of the aspects, the composition is an emulsion. In some embodiments of any of the aspects, the composition comprises an emulsion. In some embodiments of any of the aspects, the composition comprises an emulsion of one or more omega-6 fatty acids comprising at least Arachidonic acid (ARA) and one or more omega-3 fatty acids comprising at least Docosahexaenoic acid (DHA). In some embodiments of any of the aspects, the composition comprises an emulsion of one or more omega-6 fatty acids comprising at least Arachidonic acid (ARA) and one or more omega-3 fatty acids comprising at least EPA. In some embodiments of any of the aspects, the composition comprises an emulsion of one or more omega-6 fatty acids comprising at least Arachidonic acid (ARA) and one or more omega-3 fatty acids comprising at least DHA and EPA. In some embodiments of any of the aspects, the composition comprises a mixture of a) an emulsion of one or more omega-6 fatty acids comprising at least Arachidonic acid (ARA) and b) an emulsion of one or more omega-3 fatty acids comprising at least Docosahexaenoic acid (DHA). In some embodiments of any of the aspects, the composition comprises a mixture of a) an emulsion of one or more omega-6 fatty acids comprising at least Arachidonic acid (ARA) and b) an emulsion of one or more omega-3 fatty acids comprising at least EPA. In some embodiments of any of the aspects, the composition comprises a mixture of a) an emulsion of one or more omega-6 fatty acids comprising at least Arachidonic acid (ARA) and b) an emulsion of one or more omega-3 fatty acids comprising at least DHA and EPA.

As used herein, the term “emulsion” refers to a heterogeneous system comprising at least two or more substantially immiscible liquids, wherein one liquid is dispersed in another liquid in the form of droplets. By way of example only, emulsions can be biphasic systems comprising two immiscible liquid phases intimately mixed and dispersed with each other. Examples of an emulsion include, but are not limited to, water-in-oil emulsions, oil-in-water emulsion, water-in-water, water-in-oil-in-water emulsions, and oil-in-water-in-oil emulsions. In some embodiments of any of the aspects, the continuous phase of the emulsion is water.

In some embodiments of any of the aspects, the emulsion comprises water. In some embodiments, the emulsion is about 20% total fats in water by weight/volume. In some embodiments, the emulsion is from about 10% total fats in water by weight/volume to about 50% total fats in water by weight/volume. In some embodiments, the emulsion is about 20% total fats in water by weight/volume. In some embodiments, the emulsion is from 10% total fats in water by weight/volume to 50% total fats in water by weight/volume.

Emulsions are generally unstable mixtures and do not form spontaneously, thus, in order to mix the continuous and dispersed phases and form the emulsion, an energy input is required. This energy can be applied, for example, by shaking, stirring, homogenizing, spray processing, high pressure pumping and ultrasonic emulsification. The emulsion formulation described herein can be made by blending the fat components listed herein with any proteins, carbohydrates, and/or other additional additives, and homogenizing the mixture into a stable emulsion. In some embodiments of any of the aspects, the both the a) ARA and b) DHA and/or EPA are emulsified, e.g., not merely mixed or mixed into an emulsion of the other. Over time however, the emulsion formed may tend to revert to the stable state of separate oil and aqueous layers. Accordingly, in some embodiments, the emulsion formulation described herein can further comprise any natural or synthetic emulsifier known in the art. The addition of an emulsifier can increase the kinetic stability of emulsions so that, once formed, the emulsion does not change significantly in long term storage.

Emulsion as described herein can be prepared by a number of conventional techniques known to those skilled in the art. For example, the core lipid(s) are first mixed with one or more emulsifiers and any further ingredients or additives. The emulsion is then prepared by slowly adding this oil phase into water with constant agitation. If an osmolality modifier is being used, it is added to the water prior to mixture with the oil phase. The pH can be adjusted at this stage, if necessary, and the final volume adjusted with water, if required.

In some embodiments of any of the aspects, the particle size of the oil globules in the emulsion, e.g., when formulated for parenteral administration are within or below the size range of the naturally occurring chylomicron, which is 0.4-1.0 μm. If the particle size is larger than this, the lipid particles may be deposited in the liver, spleen and lungs resulting in significant fat load following infusion (Rahui C. M., et I al., Am. Hosp. Pharm. 1992, 49:2749-2755). Lipids with small particle sizes disperse better in the emulsion and tend to produce safer and more stable emulsions. Selection of appropriate conditions for the preparation of the emulsions according to the present invention is considered to be within the ordinary skills of a worker in the art.

In some embodiments of any of the aspects, the emulsion is a stable emulsion. As used herein, the term “stable emulsion” refers to an emulsion in which droplets remain substantially evenly dispersed throughout a continuous phase (or a carrier liquid) for an extended time period (e.g., at least about 1 month or longer), including reasonable storage and usage times. For example, the droplets do not aggregate or settle out after an extended time period (e.g., at least about 1 month or longer).

As used herein, the term “substantially immiscible” refers to two or more liquids that do not form a homogenous mixture when they are in contact with each other. In some embodiments, when two or more substantially immiscible liquids are in contact with each other, one of the liquids can have a partial solubility (e.g., no more than 10% or lower) in another substantially immiscible liquid. The term “homogenous mixture” as used herein means that all components and/or liquids in a mixture are readily present in a single phase. For instance, one or more of the components and/or liquids do not separate into different phases even when the mixture is left stationary for an extended period of time (e.g., at least about 6 hours or longer, including, e.g., at least about 12 hours, at least about 18 hours, at least about 24 hours, or longer). When referring to miscibility of the droplets and the carrier liquid, the term “substantially immiscible” refers to a liquid (e.g., a thin liquid layer) forming at least the outer surface of the droplets and the carrier liquid that do not form a homogenous mixture when they are in contact with each other.

As used herein and throughout the specification, the term “droplet” refers to a finite volume of matter comprising at least one liquid or at least one liquid phase, including, e.g., at least two or more liquids or liquid phases. The droplets can be of any dimension, configuration, and/or shape. In some embodiments of various aspects described herein, the droplets can have a droplet size that is smaller (e.g., at least 50% smaller) than the inner diameter of a needle that is used to administer an emulsion comprising the droplets. It will be understood by one of ordinary skill in the art that droplets usually exhibit a distribution of droplet sizes around the indicated “size.” Unless otherwise stated, the term “droplet size” or “size” as used herein refers to the mode of a size distribution of droplets, i.e., the value that occurs most frequently in the size distribution. Methods for measuring the droplet size are known to a skilled artisan, e.g., by dynamic light scattering (such as photo-correlation spectroscopy, laser diffraction, low-angle laser light scattering (LALLS), and medium-angle laser light scattering (MALLS)), light obscuration methods (such as Coulter analysis method), or other techniques (such as rheology, and light or electron microscopy).

While the compositions described herein are intended to be administered as emulsions, other forms of the formulations are also encompassed by the invention. For example, the compositions described herein as emulsions can also be made in powder form by increasing the percent total solids in the formula, using procedures well known to those skilled in the art. The concentrate or powder can be reconstituted for feeding by adding water (tap or deionized-sterilized water) to form an emulsion.

As used herein, the term “docosahexaenoic acid” or “DHA” is an omega-3 fatty acid, i.e., all-cis-docosa-4,7,10,13,16,19-hexa-enoic acid or 22:6(ω-3).

As used herein, the term “eicosapentaenoic acid” or “EPA” is an omega-3 fatty acid, i.e., (5Z,8Z,11Z,14Z,17Z)-Icosa-5,8,11,14,17-pentaenoic acid or 20:5(ω-3).

As used herein, the term “arachidonic acid” or “ARA” is an omega-6 fatty acid, i.e., cis-5,8,11,14-eicosatetraenoic acid or 20:4(ω-6).

As used herein, the term “fatty acid” includes fatty acids such as unsaturated (e.g., monounsaturated, polyunsaturated) or saturated fatty acids, as well as pharmaceutically-acceptable esters, free acids, mono-, di- and triglycerides, derivatives, conjugates, precursors, salts, and mixtures thereof.

As used herein, the term “omega-6 fatty acids” includes natural and synthetic omega-6 fatty acids, as well as pharmaceutically acceptable esters, free acids, triglycerides, derivatives, conjugates, precursors, salts, and mixtures thereof. Omega-6 fatty acids can include, but are not limited to, Linolenic acid (LA); Gamma-linolenic acid (GLA); calendic acid; eicosadienoic acid; dihomo-gamma-linolenic acids (DGLA); arachidonic acid (ARA); docosadienoic acid; adrenic acid; osbond acid; tetracosatetraenoic acid; and tetracosapentaenoic acid. The omega-6 fatty acids can be provided in the form of an omega-6 oil. Omega-6 oils for use in the composition (e.g., the emulsion) s described herein can have a high content of ARA. The omega-6 oils and/or omega-6 fatty acids may be from animal, plant, algal, or synthetic origin. In some embodiments of any of the aspects, the omega-6 oils and/or omega-6 fatty acids may be from plant, algal, or synthetic origin. For example, suitable sources of omega-6 oils and/or omega-6 fatty acids are flaxseed oil, canola oil, mustard seed oil, coconut oil, olive oil, soybean oil, Mortierella alpina oil, and the like.

In some embodiments of any of the aspects, the omega-6 fatty acids of a composition (e.g., an emulsion) described herein can comprise, consist of, or consist essentially of ARA.

Examples of omega-6 fatty acids and mixtures thereof encompassed by the present disclosure include the omega-6 fatty acids as defined in CAS 506-32-1; GRAS Notices GRN 730, GRN 326, GRN 94, GRN 80, GRN 41 (see fda.gov/media/112256/download); and FSANZ (Food Standards Australia New Zealand) 2003. DHASCO and ARASCO oils as sources of long-chain polyunsaturated fatty acids in infant formula: A safety assessment. Technical report series 22:1-54. Available on the world wide web at foodstandards.gov.au; which are incorporated by reference herein in their entireties.

In some embodiments of any of the aspects, the ARA and/or omega-6 fatty acids are provided in the form of ARASCO™ (DSM; Heerlen, Netherlands). In some embodiments of any of the aspects, the compositions described herein comprise ARASCO™. Suitable algal oils are also available from Cargill, Fementlag (e.g., DHA ORIGINS™), and Corbion (e.g, ALGAPRIME™). Additional commercial examples of omega-6 fatty acids suitable for the present disclosure comprise different fatty acid mixtures (e.g., that can be in the form of triglycerides (TG), ethyl esters (EE), free fatty acid form (FA) and/or as phospholipids) including, but not limited to omega-6 fatty acid preparations available from Linyi Youkang Biology Co, Yukang Biotechnology Co. Ltd., Alfrebro LLC, Archer Daniels Midland Co., BOC Sciences, Best of Chemicals Supplier, Jiangyin Healthway International Trade Co., Ltd., Penta International Corporation, TCI AMERICA, and Qingdao Free Trade Zone United International Co Ltd.

The omega-6 fatty acids used herein can be purified, e.g., to meet the quality standards for parenteral administration. In some embodiments of any of the aspects, the omega-6 fatty acids can be enriched with additional or further omega-6 fatty acids, e.g., purified or synthesized omega-6 fatty acids from another source can be added to the omega-6 fatty acids to increase the omega-6 fatty acid content. Methods of extracting and refining oils are well known in the art. It is not necessary for the oils to undergo re-esterification in order to purify, extract, or refine them.

In some embodiments of any of the aspects, the composition comprises plant or fungal oil (e.g., an omega-6 oil). In some embodiments of any of the aspects, the composition comprises plant or fungal oil comprising ARA. In some embodiments of any of the aspects, the ARA of the composition is provided in the form of a plant or fungal oil. In some embodiments of any of the aspects, the plant or fungal oil is not distilled or re-esterified. In some embodiments of any of the aspects, the total triglyceride+diglyceride content of the plant or fungal oil comprises no more than 10% diglyceride. In some embodiments of any of the aspects, the plant or fungal oil comprises no diglyceride. In some embodiments of any of the aspects, the plant or fungal oil comprises no more than 5% (w/w) sterols, no more than 70 mg/L phytosterols, no more than 4% (w/w) stigmasterol, and/or no more than 10 mg/L stigmasterol. In some embodiments of any of the aspects, the plant oil is, comprises, consists of, or consists essentially of soybean oil or olive oil. In some embodiments of any of the aspects, the fungal oil is, comprises, consists of, or consists essentially of Mortierella alpina oil.

As shown in Example 2 herein, formulations such as INTRALIPID exhibit significant toxicity (e.g., liver injury) as compared to the compositions described herein. The experiments in Example 2, which contrast soybean oil with low linoleic content with INTRALIPID indicate that linoleic acid and/or phytosterols are a significant source of the toxicity of omega-6 fatty acids such as soybean oil or INTRALIPID. Accordingly, in some embodiments, the composition (or components thereof, e.g., a) DHA and/or EPA or b) ARA) described herein comprises no more than 40% w/v linoleic acid. In some embodiments, the composition (or components thereof, e.g., a) DHA and/or EPA or b) ARA) described herein comprises no more than 30% w/v linoleic acid. In some embodiments, the composition (or components thereof, e.g., a) DHA and/or EPA or b) ARA) described herein comprises no more than 20% w/v linoleic acid. In some embodiments, the composition (or components thereof, e.g., a) DHA and/or EPA or b) ARA) described herein comprises no more than 10% w/v linoleic acid. In some embodiments, the composition (or components thereof, e.g., a) DHA and/or EPA or b) ARA) described herein comprises no more than 5% w/v linoleic acid. In some embodiments of any of the aspects, the plant or fungal oil does not comprise linoleic acid. In some embodiments of any of the aspects, a composition described herein does not comprise linoleic acid.

In some embodiments, the composition (or components thereof, e.g., a) DHA and/or EPA or b) ARA) described herein comprises, or is administered at a dose of no more than 621 mg/kg/day of linoleic acid. In some embodiments, the composition (or components thereof, e.g., a) DHA and/or EPA or b) ARA) described herein comprises, or is administered at a dose of no more than 401 mg/kg/day of linoleic acid. In some embodiments, the composition (or components thereof, e.g., a) DHA and/or EPA or b) ARA) described herein comprises, or is administered at a dose of no more than 287 mg/kg/day of linoleic acid.

INTRALIPID is utilized at a dose that provides ~2-3 g of fat per kg/day, with approximately 55% of the total fatty acids being linoleic acid. SMOFLIPID is utilized at a dose that provides ~1 g of fat per kg/day. These levels are demonstrated to exhibit liver toxicity. Accordingly, in some embodiments of any of the aspects, described herein is a formulation or method that relates to a dose that provides less than 1 g of linoleic acid per kg/day. In some embodiments of any of the aspects, described herein is a formulation or method that relates to a dose that provides less than 500 mg of linoleic acid per kg/day. in some embodiments of any of the aspects, described herein is a formulation or method that relates to a dose that provides less than 400 mg of linoleic acid per kg/day. In some embodiments of any of the aspects, described herein is a formulation or method that relates to a dose that provides less than 300 mg of linoleic acid per kg/day.

As used herein, the term “omega-3 fatty acids” includes natural and synthetic omega-3 fatty acids, as well as pharmaceutically acceptable esters, free acids, triglycerides, derivatives, conjugates, precursors, salts, and mixtures thereof. Omega-3 fatty acids can include, but are not limited to, hexadecatrienoic acid (HTA); α-Linolenic acid (ALA); Stearidonic acid (SDA); Eicosatrienoic acid (ETE); Eicosatetraenoic acid (ETA); Eicosapentaenoic acid (EPA); Heneicosapentaenoic acid (HPA); Docosapentaenoic acid (DPA); Clupanodonic acid; Docosahexaenoic acid (DHA); Tetracosapentaenoic acid; and Tetracosahexaenoic acid (Nisinic acid). Omega-3 fatty acids for use in the composition (e.g., the emulsion) s described herein can have a high content of eicosapentaenoic acid (EPA) as well as docosahexaenoic acid (DHA). The omega-3 fatty acids can be provided in the form of an omega-3 oil. The omega-3 oils and/or omega-3 fatty acids may be from animal origin. The omega-3 oils and/or omega-3 fatty acids may be from marine or synthetic origin. For example, a suitable source of omega-3 oils and/or omega-3 fatty acids is fish or seal oil. Suitable fish oil sources include deep-sea fish, shark, salmon, cod, salmon, bonito, mackerel, Atlantic mackerel, haddock, herring, mahi mahi, menhaden, mackerel, caplin, tilapia, pacific saury, krill, anchovies, pollock, trout, whitefish, tuna, smelt, shad, and sardines, cold-water fish as described elsewhere herein, and the like.

The fatty acid(s) according to the present disclosure may be derived from animal oils and/or non-animal oils. In some embodiments of the present disclosure, the fatty acid(s) are derived from at least one oil chosen from marine oil, algae oil, fungal oil, plant-based oil, and microbial oil. Marine oils include, for example, fish oil, such as tuna fish oil, krill oil, and lipid composition derived from fish. Plant-based oils include, for example, flaxseed oil, canola oil, mustard seed oil, olive oil, and soybean oil. Microbial oils include, for example, products by Martek. Fungal and algal oils incude, for example, products by DSM.

In some embodiments of any of the aspects, the omega-3 fatty acids of a composition (e.g., an emulsion) described herein can comprise, consist of, or consist essentially of DHA. In some embodiments of any of the aspects, the omega-3 fatty acids of a composition (e.g., an emulsion) described herein can comprise, consist of, or consist essentially of EPA. In some embodiments of any of the aspects, the omega-3 fatty acids of a composition (e.g., an emulsion) described herein can comprise, consist of, or consist esstentially of EPA and DHA.

Examples of omega-3 fatty acids and mixtures thereof encompassed by the present disclosure include the omega-3 fatty acids as defined in the European Pharmacopoeia Omega-3 Triglycerides, the European Pharmacopoeia Omega-3 acid Ethyl Esters 60, or the Fish oil rich in omega-3 acids monograph; which are incorporated by reference herein in their entireties.

In some embodiments of any of the aspects, the DHA, EPA, and/or omega-3 fatty acids are provided in the form of DHASCO™ (DSM; Heerlen, Netherlands). In some embodiments of any of the aspects, the compositions described herein comprise DHASCO™.

Additional commercial examples of omega-3 fatty acids suitable for the present disclosure comprise different fatty acid mixtures (e.g., that can be in the form of triglycerides (TG), ethyl esters (EE), free fatty acid form (FA) and/or as phospholipids) including, but not limited to: Incromega™ omega-3 marine oil concentrates such as Incromega™ E1070, Incromega™ TG7010 SR, Incromega™ E7010 SR, Incromega™ TG6015, Incromega™ EPA500TG SR, Incromega™ E400200 SR, Incromega™ E4010, Incromega™ DHA700TG SR, Incromega™ DHA 700E SR, Incromega™ DHA500TG SR, Incromega™ TG3322 SR, Incromega™ E3322 SR, Incromega™ TG3322, Incromega™ E3322, Incromega™ Trio TG/EE (Croda International PLC, Yorkshire, England); EPAX6000FA, EPAX5000TG, EPAX4510TG, EPAX2050TG, EPAX7010EE, EPAX5500EE, EPAX5500TG, EPAX5000EE, EPAX5000TG, EPAX6000EE, EPAX6000TG, EPAX6000FA, EPAX6500EE, EPAX6500TG, EPAX4510TG, EPAX1050TG, EPAX2050TG, EPAX 7010TG, EPAX7010EE, EPAX6015TG/EE, EPAX4020TG, and EPAX4020EE (EPAX is a wholly-owned subsidiary of Norwegian company Austevoll Seafood ASA); MEG-3® EPA/DHA fish oil concentrates (Ocean Nutrition Canada); DHA FNO “Functional Nutritional Oil” and DHA CL “Clear Liquid” (Lonza); Superba™ Krill Oil (Aker); omega-3 products comprising DHA produced by Martek; Neptune krill oil (Neptune); cod-liver oil products and anti-reflux fish oil concentrate (TG) produced by Møllers; Lysi Omega-3 Fish oil; Seven Seas Triomega® Cod Liver Oil Blend (Seven Seas); and Fri Flyt Omega-3 (Vesterålens).

The omega-3 fatty acids used herein can be purified, e.g., to meet the quality standards for parenteral administration. In some embodiments of any of the aspects, the omega-3 fatty acids can be enriched with additional or further omega-3 fatty acids, e.g., purified or synthesized omega-3 fatty acids from another source can be added to the omega-3 fatty acids to increase the omega-3 fatty acid content. Methods of extracting and refining oils are well known in the art. It is not necessary for the oils to undergo re-esterification in order to purify, extract, or refine them.

In some embodiments of any of the aspects, wherein the composition comprises a fish oil, and/or the DHA is provided in the form of fish oil. As used herein, “fish oil” refers to an oil derived from a fish or fish tissue. Fish oil is available commercially, for example 10% (wt/wt) fish oil triglycerides can be obtained from Nisshin Flour Milling Co. located in Nisshin, Japan. OMEGAVEN (Fresnius Kabi) is suitable for use in the methods and compositions described herein. In some embodiments of any of the aspects, the fish oil can comprise omega-3 fatty acids, DHA, and/or EPA. In some embodiments of any of the aspects, a fish oil can be a fish oil derived from one or more cold-water fish, which are known for having high omega-3 fatty acid content. Non-limiting examples of cold water fish can include a deep-sea fish, shark, salmon, cod, salmon, bonito, mackerel, Atlantic mackerel, haddock, herring, mahi mahi, menhaden, mackerel, caplin, tilapia, pacific saury, krill, anchovies, pollock, trout, whitefish, tuna, smelt, shad, and sardines. In some embodiments of any of the aspects, a fish oil can be a fish oil derived from one or more saltwater cold-water fish. In some embodiments of any of the aspects, a fish oil can be a fish oil derived from shark, salmon, cod, salmon, bonito, mackerel, Atlantic mackerel, haddock, herring, mahi mahi, menhaden, mackerel, caplin, tilapia, pacific saury, krill, anchovies, pollock, trout, whitefish, tuna, smelt, shad, sardines, or any combination thereof. In some embodiments of any of the aspects, the fish oil is not distilled or re-esterified. In some embodiments of any of the aspects, the total triglyceride+diglyceride content of the fish oil comprises no more than 10% diglyceride. In some embodiments of any of the aspects, the fish oil comprises no diglyceride. In some embodiments of any of the aspects, the fish oil comprises no more than 5% (w/w) sterols, no more than 70 mg/L phytosterols, no more than 4% (w/w) stigmasterol, and/or no more than 10 mg/L stigmasterol.

In some embodiments of any of the aspects, the composition comprises an algal oil, and/or the DHA and/or EPA is provided in the form of algal oil. In some embodiments of any of the aspects,

In some embodiments of any of the aspects, the algal oil is, comprises, consists of, or consists essentially of oil from Crypthecodinium cohnii. In some embodiments of any of the aspects, the algal oil is not distilled or re-esterified. In some embodiments of any of the aspects, the total triglyceride+diglyceride content of the algal oil comprises no more than 10% diglyceride. In some embodiments of any of the aspects, the algal oil comprises no diglyceride. In some embodiments of any of the aspects, the algal oil comprises no more than 5% (w/w) sterols, no more than 70 mg/L phytosterols, no more than 4% (w/w) stigmasterol, and/or no more than 10 mg/L stigmasterol.

In some embodiments of any of the aspects, the composition comprises a) plant oil which is, comprises, consists of, or consists essentially of soybean oil and b) an algal oil which is, comprises, consists of, or consists essentially of oil from Crypthecodinium cohnii. In some embodiments of any of the aspects, the composition does not comprise a source of omega-6 fatty acids or omega-3 fatty acids other than the plant oil and algal oil. In some embodiments of any of the aspects, the composition comprises a) plant oil which is, comprises, consists of, or consists essentially of soybean oil and b) a fish oil. In some embodiments of any of the aspects, the composition does not comprise a source of omega-6 fatty acids or omega-3 fatty acids other than the plant oil and fish oil.

In some embodiments of any of the aspects, the composition comprises a) plant oil which is, comprises, consists of, or consists essentially of olive oil and b) an algal oil which is, comprises, consists of, or consists essentially of oil from Crypthecodinium cohnii. In some embodiments of any of the aspects, the composition does not comprise a source of omega-6 fatty acids or omega-3 fatty acids other than the plant oil and algal oil. In some embodiments of any of the aspects, the composition comprises a) plant oil which is, comprises, consists of, or consists essentially of olive oil and b) a fish oil. In some embodiments of any of the aspects, the composition does not comprise a source of omega-6 fatty acids or omega-3 fatty acids other than the plant oil and fish oil.

In some embodiments of any of the aspects, the composition comprises a) fungal oil which is, comprises, consists of, or consists essentially of Mortierella alpina oil and b) an algal oil which is, comprises, consists of, or consists essentially of oil from Crypthecodinium cohnii. In some embodiments of any of the aspects, the composition does not comprise a source of omega-6 fatty acids or omega-3 fatty acids other than the fungal oil and algal oil. In some embodiments of any of the aspects, the composition comprises a) fungal oil which is, comprises, consists of, or consists essentially of Mortierella alpina oil and and b) a fish oil. In some embodiments of any of the aspects, the composition does not comprise a source of omega-6 fatty acids or omega-3 fatty acids other than the fungal oil and fish oil.

In some embodiments of any of the aspects, the composition does not comprise a source of omega-6 fatty acids or omega-3 fatty acids other than the fungal oil and algal oil.

In some embodiments of any of the aspects, the a) ARA and b) DHA and/or EPA present in a composition described herein are present in certain ratios, e.g., weight ratios.

In some embodiments of any of the aspects, the ratio of a) ARA and b) DHA and/or EPA is from about 1:1 to about 1:20 by weight. In some embodiments of any of the aspects, the ratio of a) ARA and b) DHA and/or EPA is from about 1:1 to about 1:15 by weight. In some embodiments of any of the aspects, the ratio of a) ARA and b) DHA and/or EPA is from about 1:2 to about 1:15 by weight. In some embodiments of any of the aspects, the ratio of a) ARA and b) DHA and/or EPA is from about 1:2.8 to about 1:12.5 by weight. In some embodiments of any of the aspects, the ratio of a) ARA and b) DHA and/or EPA is from about 3:2 to about 3:5 by weight. In some embodiments of any of the aspects, the ratio of a) ARA and b) DHA and/or EPA is from about 1:2 to about 3:5 by weight. In some embodiments of any of the aspects, the ratio of a) ARA and b) DHA and/or EPA is from about 1:2 to about 1:5 by weight. In some embodiments of any of the aspects, the ratio of a) ARA and b) DHA and/or EPA is from about 2:1 to about 1:4 by weight. In some embodiments of any of the aspects, the ratio of a) ARA and b) DHA and/or EPA is from about 1:1 to about 1:2 by weight.

In some embodiments of any of the aspects, the ratio of a) ARA and b) DHA and/or EPA is from 3:2 to 3:5 by weight. In some embodiments of any of the aspects, the ratio of a) ARA and b) DHA and/or EPA is from 1:2 to 3:5 by weight. In some embodiments of any of the aspects, the ratio of a) ARA and b) DHA and/or EPA is from 1:2 to 1:5 by weight. In some embodiments of any of the aspects, the ratio of a) ARA and b) DHA and/or EPA is from 2:1 to 1:4 by weight. In some embodiments of any of the aspects, the ratio of a) ARA and b) DHA and/or EPA is from 1:1 to 1:2 by weight.

In some embodiments of any of the aspects, the compositions described herein comprise further omega-6 fatty acids and/or omega-3 fatty acids, e.g, omega-6 fatty acids and/or omega-3 fatty acids in addition to a) ARA and b) DHA and/or EPA. In some embodiments of any of the aspects, the ARA and DHA and/or EPA comprise at least 10% of the total omega-3 fatty acids and omega-6 fatty acids. In some embodiments of any of the aspects, the ARA and DHA and/or EPA comprise at least 20% of the total omega-3 fatty acids and omega-6 fatty acids. In some embodiments of any of the aspects, the ARA and DHA and/or EPA comprise at least 30% of the total omega-3 fatty acids and omega-6 fatty acids. In some embodiments of any of the aspects, the ARA and DHA and/or EPA comprise at least 35% of the total omega-3 fatty acids and omega-6 fatty acids. In some embodiments of any of the aspects, the ARA and DHA and/or EPA comprise at least 60% of the total omega-3 fatty acids and omega-6 fatty acids. In some embodiments of any of the aspects, the ARA and DHA and/or EPA comprise at least 70% of the total omega-3 fatty acids and omega-6 fatty acids. In some embodiments of any of the aspects, the ARA and DHA and/or EPA comprise at least 80% of the total omega-3 fatty acids and omega-6 fatty acids. In some embodiments of any of the aspects, the ARA and DHA and/or EPA comprise at least 90% of the total omega-3 fatty acids and omega-6 fatty acids.

In some embodiments of any of the aspects, more than 20% (by weight) of the total omega-3 fatty acid present in the composition is DHA. In some embodiments of any of the aspects, more than 25% (by weight) of the total omega-3 fatty acid present in the composition is DHA.

In some embodiments of any of the aspects, more than 30% (by weight) of the total omega-3 fatty acid present in the composition is DHA. In some embodiments of any of the aspects, more than 35% (by weight) of the total omega-3 fatty acid present in the composition is DHA. In some embodiments of any of the aspects, more than 40% (by weight) of the total omega-3 fatty acid present in the composition is DHA. In some embodiments of any of the aspects, more than 45% (by weight) of the total omega-3 fatty acid present in the composition is DHA.

In some embodiments of any of the aspects, DHA is the predominate omega-3 fatty acid in the composition. In some embodiments of any of the aspects, there is more DHA by weight present in the composition than any other omega-3 fatty acid. In some embodiments of any of the aspects, more than 50% (by weight) of the total omega-3 fatty acid present in the composition is DHA.

In some embodiments of any of the aspects, more than 20% (by weight) of the total omega-3 fatty acid present in the composition is EPA. In some embodiments of any of the aspects, more than 25% (by weight) of the total omega-3 fatty acid present in the composition is EPA.

In some embodiments of any of the aspects, more than 30% (by weight) of the total omega-3 fatty acid present in the composition is EPA. In some embodiments of any of the aspects, more than 35% (by weight) of the total omega-3 fatty acid present in the composition is EPA. In some embodiments of any of the aspects, more than 40% (by weight) of the total omega-3 fatty acid present in the composition is EPA. In some embodiments of any of the aspects, more than 45% (by weight) of the total omega-3 fatty acid present in the composition is EPA.

In some embodiments of any of the aspects, EPA is the predominate omega-3 fatty acid in the composition. In some embodiments of any of the aspects, there is more EPA by weight present in the composition than any other omega-3 fatty acid. In some embodiments of any of the aspects, more than 50% (by weight) of the total omega-3 fatty acid present in the composition is EPA.

In some embodiments of any of the aspects, more than 20% (by weight) of the total omega-3 fatty acid present in the composition is DHA and EPA. In some embodiments of any of the aspects, more than 25% (by weight) of the total omega-3 fatty acid present in the composition is DHA and EPA.

In some embodiments of any of the aspects, more than 30% (by weight) of the total omega-3 fatty acid present in the composition is DHA and EPA. In some embodiments of any of the aspects, more than 35% (by weight) of the total omega-3 fatty acid present in the composition is DHA and EPA. In some embodiments of any of the aspects, more than 40% (by weight) of the total omega-3 fatty acid present in the composition is DHA and EPA. In some embodiments of any of the aspects, more than 45% (by weight) of the total omega-3 fatty acid present in the composition is DHA and EPA

In some embodiments of any of the aspects, more than 20% (by weight) of the total omega-6 fatty acid present in the composition is ARA. In some embodiments of any of the aspects, more than 25% (by weight) of the total omega-6 fatty acid present in the composition is ARA. In some embodiments of any of the aspects, more than 30% (by weight) of the total omega-6 fatty acid present in the composition is ARA. In some embodiments of any of the aspects, more than 35% (by weight) of the total omega-6 fatty acid present in the composition is ARA. In some embodiments of any of the aspects, more than 40% (by weight) of the total omega-6 fatty acid present in the composition is ARA. In some embodiments of any of the aspects, more than 45% (by weight) of the total omega-6 fatty acid present in the composition is ARA.

In some embodiments of any of the aspects, ARA is the predominant omega-6 fatty acid in the composition. In some embodiments of any of the aspects, there is more ARA by weight present in the composition than any other omega-6 fatty acid. In some embodiments of any of the aspects, more than 50% (by weight) of the total omega-6 fatty acid present in the composition is ARA.

In some embodiments of any of the aspects, the a) ARA and b) DHA and/or EPA are, collectively, at least 50% of the total omega-3 fatty acids and omega-6 fatty acids present in the composition, e.g., by weight. In some embodiments of any of the aspects, the a) ARA and b) DHA and/or EPA are, collectively, at least 60% of the total omega-3 fatty acids and omega-6 fatty acids present in the composition, e.g., by weight. In some embodiments of any of the aspects, the a) ARA and b) DHA and/or EPA are, collectively, at least 65% of the total omega-3 fatty acids and omega-6 fatty acids present in the composition, e.g., by weight. In some embodiments of any of the aspects, the a) ARA and b) DHA and/or EPA are, collectively, at least 70% of the total omega-3 fatty acids and omega-6 fatty acids present in the composition, e.g., by weight. In some embodiments of any of the aspects, the a) ARA and b) DHA and/or EPA are, collectively, at least 75% of the total omega-3 fatty acids and omega-6 fatty acids present in the composition, e.g., by weight. In some embodiments of any of the aspects, the a) ARA and b) DHA and/or EPA are, collectively, at least 80% of the total omega-3 fatty acids and omega-6 fatty acids present in the composition, e.g., by weight. In some embodiments of any of the aspects, the a) ARA and b) DHA and/or EPA are, collectively, at least 85% of the total omega-3 fatty acids and omega-6 fatty acids present in the composition, e.g., by weight. In some embodiments of any of the aspects, the a) ARA and b) DHA and/or EPA are, collectively, at least 90% of the total omega-3 fatty acids and omega-6 fatty acids present in the composition, e.g., by weight. In some embodiments of any of the aspects, the a) ARA and b) DHA and/or EPA are, collectively, at least 95% of the total omega-3 fatty acids and omega-6 fatty acids present in the composition, e.g., by weight.

As shown in Example 2 herein, the omega 6:omega 3 ratio of a composition correlates with the proinflammatory versus anti-inflammatory effects of the composition. Accordingly, in some embodiments of any of the aspects, the ratio of omega-6 fatty acids to omega-3 fatty acids in the composition is from about 2:1 to about 1:1 by weight. In some embodiments of any of the aspects, the ratio of omega-6 fatty acids to omega-3 fatty acids in the composition is from about 3:2 to about 3:5 by weight. In some embodiments of any of the aspects, the ratio of omega-6 fatty acids to omega-3 fatty acids in the composition is from about 1:2 to about 3:5 by weight. In some embodiments of any of the aspects, the ratio of omega-6 fatty acids to omega-3 fatty acids in the composition is from about 1:2 to about 1:5 by weight. In some embodiments of any of the aspects, the ratio of omega-6 fatty acids to omega-3 fatty acids in the composition is from about 2:1 to about 1:4 by weight. In some embodiments of any of the aspects, the ratio of omega-6 fatty acids to omega-3 fatty acids in the composition is from about 1:1 to about 1:2 by weight. In some embodiments of any of the aspects, the ratio of omega-6 fatty acids to omega-3 fatty acids in the composition is from about 8.41:1 to about 1:11.1 by weight. In some embodiments of any of the aspects, the ratio of omega-6 fatty acids to omega-3 fatty acids in the composition is from about 8.41:1 to about 1.44:1 by weight. In some embodiments of any of the aspects, the ratio of omega-6 fatty acids to omega-3 fatty acids in the composition is from about 8.41:1 to about 1:1.27 by weight. In some embodiments of any of the aspects, the ratio of omega-6 fatty acids to omega-3 fatty acids in the composition is from about 8.41:1 to about 1:2.17 by weight. In some embodiments of any of the aspects, the ratio of omega-6 fatty acids to omega-3 fatty acids in the composition is from about 2:1 to about 1:11.1 by weight. In some embodiments of any of the aspects, the ratio of omega-6 fatty acids to omega-3 fatty acids in the composition is from about 2:1 to about 1.44:1 by weight. In some embodiments of any of the aspects, the ratio of omega-6 fatty acids to omega-3 fatty acids in the composition is from about 2:1 to about 1:1.27 by weight. In some embodiments of any of the aspects, the ratio of omega-6 fatty acids to omega-3 fatty acids in the composition is from about 2:1 to about 1:2.17 by weight. In some embodiments of any of the aspects, the ratio of omega-6 fatty acids to omega-3 fatty acids in the composition is from about 8.41:1 to about 1:4 by weight. In some embodiments of any of the aspects, the ratio of omega-6 fatty acids to omega-3 fatty acids in the composition is from about 1:1 to about 1:11.1 by weight. In some embodiments of any of the aspects, the ratio of omega-6 fatty acids to omega-3 fatty acids in the composition is from about 1:1 to about 1.44:1 by weight. In some embodiments of any of the aspects, the ratio of omega-6 fatty acids to omega-3 fatty acids in the composition is from about 1:1 to about 1:1.27 by weight. In some embodiments of any of the aspects, the ratio of omega-6 fatty acids to omega-3 fatty acids in the composition is from about 8.41:1 to about 1:2 by weight. In some embodiments of any of the aspects, the ratio of omega-6 fatty acids to omega-3 fatty acids in the composition is from about 1.44:1 to about 1:11.1 by weight. In some embodiments of any of the aspects, the ratio of omega-6 fatty acids to omega-3 fatty acids in the composition is from about 1.44:1 to about 1:1.27 by weight. In some embodiments of any of the aspects, the ratio of omega-6 fatty acids to omega-3 fatty acids in the composition is from about 1.44:1 to about 1:2.17 by weight. In some embodiments of any of the aspects, the ratio of omega-6 fatty acids to omega-3 fatty acids in the composition is from about 1:1.27 to about 1:2.17 by weight.

In some embodiments of any of the aspects described herein, the omega-3 and/or omega-6 fatty acids can be highly refined, e.g., highly enriched beyond the initial content of omega-3 and/or omega-6 fatty acids and/or their triglycerin compound as part of this specific procedure. In some embodiments of any of the aspects, these compositions can comprise a minimum of 95% weight, e.g., 96% weight, 97% weight, 98% weight or greater, of monomeric triglycerides. In some embodiments of any of the aspects, these compositions can comprise less than 1% weight percent of oxidized triglycerides, less than 0.2% weight percent of trimeric and oligomeric triglycerides and less than 0.8% weight percent of dimeric polyglycerides, and less than 1.5% weight, of unemulsifiable particularly carbohydrates and sterane. In some embodimens of any of the aspects, the cholesterol content of the omega-3 and/or omega-6 fatty acids and/or the composition described herein (e.g., after refinement or enrichment) comprises less than 2500 ppm, e.g., less than 1500 ppm cholesterol.

In some embodiments of any of the aspects, the omega-3 and/or omega-6 fatty acids of the compositions described herein are natural triglycerides, e.g., they have not been distilled or re-esterified. Distillation and/or re-esterification of trigylcerides is utilized in certain compositions known in the art (e.g., to remove mysristic or pamitoleic acids), but it is specifically contemplated herein that such procedures alter the triglycerides to induce toxicity therein and increase the presence of diglyceride, monoglyceride, and free fatty acids.

In some embodiments of any of the aspects, the omega-3 and/or omega-6 fatty acids of the compositions described herein are triglycerides, e.g, they are not diglycerides as described elsewhere. In some embodiments of any of the aspects, the omega-3 fatty acids of the compositions described herein are triglycerides. In some embodiments of any of the aspects, the omega-6 fatty acids of the compositions described herein are triglycerides. In some embodiments of any of the aspects, the DHA of the compositions described herein are triglycerides. In some embodiments of any of the aspects, the ARA of the compositions described herein are triglycerides.

In some embodiments of any of the aspects, the omega-3 and/or omega-6 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the omega-3 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the omega-6 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the DHA of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the EPA of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the DHA and EPA, collectively, of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the ARA of the compositions described herein comprise no more than 10%, no more than 9%, no more than 8%, no more than 7%, no more than 6%, no more than 5%, no more than 4%, no more than 3%, no more than 2%, no more than 1%, or less, or no diglycerides, e.g., as a proportion of the total diglyceride+triglyceride content. In some embodiments of any of the aspects, the total diglyceride+triglyceride content of the compositions described herein comprises no more than 10%, no more than 9%, no more than 8%, no more than 7%, no more than 6%, no more than 5%, no more than 4%, no more than 3%, no more than 2%, no more than 1%, or less, or no diglycerides.

In some embodiments of any of the aspects, the omega-3 and/or omega-6 fatty acids of the compositions described herein comprise no more than 10% diglycerides, e.g., as a proportion of the total diglyceride+triglyceride content. In some embodiments of any of the aspects, the omega-3 fatty acids of the compositions described herein comprise no more than 10% diglycerides, e.g., as a proportion of the total diglyceride+triglyceride content. In some embodiments of any of the aspects, the omega-6 fatty acids of the compositions described herein comprise no more than 10% diglycerides, e.g., as a proportion of the total diglyceride+triglyceride content. In some embodiments of any of the aspects, the DHA of the compositions described herein comprise no more than 10% diglycerides, e.g., as a proportion of the total diglyceride+triglyceride content. In some embodiments of any of the aspects, the EPA of the compositions described herein comprise no more than 10% diglycerides, e.g., as a proportion of the total diglyceride+triglyceride content. In some embodiments of any of the aspects, the DHA and EPA, collectively, of the compositions described herein comprise no more than 10% diglycerides, e.g., as a proportion of the total diglyceride+triglyceride content. In some embodiments of any of the aspects, the ARA of the compositions described herein comprise no more than 10% diglycerides, e.g., as a proportion of the total diglyceride+triglyceride content. In some embodiments of any of the aspects, the total diglyceride+triglyceride content of the compositions described herein comprises no more than 10% diglycerides.

In some embodiments of any of the aspects, the omega-3 and/or omega-6 fatty acids of the compositions described herein comprise no diglycerides. In some embodiments of any of the aspects, the omega-3 fatty acids of the compositions described herein comprise no diglycerides. In some embodiments of any of the aspects, the omega-6 fatty acids of the compositions described herein comprise no diglycerides. In some embodiments of any of the aspects, the DHA of the compositions described herein comprise no diglycerides. In some embodiments of any of the aspects, the EPA of the compositions described herein comprise no diglycerides. In some embodiments of any of the aspects, the DHA and EPA, collectively, of the compositions described herein comprise no diglycerides. In some embodiments of any of the aspects, the ARA of the compositions described herein comprise no diglycerides. In some embodiments of any of the aspects, the compositions described herein comprise no diglycerides.

In some embodiments of any of the aspects, the omega-3 and/or omega-6 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the omega-3 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the omega-6 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the DHA of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the EPA of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the DHA and EPA, collectively, of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the ARA of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides, e.g., as a proportion of the total monoglyceride+diglyceride+triglyceride content. In some embodiments of any of the aspects, the total monoglyceride+diglyceride+triglyceride content of the compositions described herein comprises no more than 10%, no more than 9%, no more than 8%, no more than 7%, no more than 6%, no more than 5%, no more than 4%, no more than 3%, no more than 2%, no more than 1%, or less, or no monoglycerides.

In some embodiments of any of the aspects, the omega-3 and/or omega-6 fatty acids of the compositions described herein comprise no more than 10% monoglycerides, e.g., as a proportion of the total monoglyceride+diglyceride+triglyceride content. In some embodiments of any of the aspects, the omega-3 fatty acids of the compositions described herein comprise no more than 10% monoglycerides, e.g., as a proportion of the total monoglyceride+diglyceride+triglyceride content. In some embodiments of any of the aspects, the omega-6 fatty acids of the compositions described herein comprise no more than 10% monoglycerides, e.g., as a proportion of the total monoglyceride+diglyceride+triglyceride content. In some embodiments of any of the aspects, the DHA of the compositions described herein comprise no more than 10% monoglycerides, e.g., as a proportion of the total monoglyceride+diglyceride+triglyceride content. In some embodiments of any of the aspects, the EPA of the compositions described herein comprise no more than 10% monoglycerides, e.g., as a proportion of the total monoglyceride+diglyceride+triglyceride content. In some embodiments of any of the aspects, the DHA and EPA, collectively, of the compositions described herein comprise no more than 10% monoglycerides, e.g., as a proportion of the total monoglyceride+diglyceride+triglyceride content. In some embodiments of any of the aspects, the ARA of the compositions described herein comprise no more than 10% monoglycerides, e.g., as a proportion of the total monoglyceride+diglyceride+triglyceride content. In some embodiments of any of the aspects, the total monoglyceride+diglyceride+triglyceride content of the compositions described herein comprises no more than 10% monoglycerides.

In some embodiments of any of the aspects, the omega-3 and/or omega-6 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the omega-3 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the omega-6 fatty acids of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the DHA of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the EPA of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the DHA and EPA, collectively, of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the ARA of the compositions described herein comprise no more than 10%, no more than 9%, no more than 8%, no more than 7%, no more than 6%, no more than 5%, no more than 4%, no more than 3%, no more than 2%, no more than 1%, or less, or no monoglycerides+diglycerides, e.g., as a proportion of the total monoglyceride+diglyceride+triglyceride content. In some embodiments of any of the aspects, the total monoglyceride+diglyceride+triglyceride content of the compositions described herein comprises no more than 10%, no more than 9%, no more than 8%, no more than 7%, no more than 6%, no more than 5%, no more than 4%, no more than 3%, no more than 2%, no more than 1%, or less, or no monoglycerides+diglycerides.

In some embodiments of any of the aspects, the omega-3 and/or omega-6 fatty acids of the compositions described herein comprise no more than 10% monoglycerides+diglycerides, e.g., as a proportion of the total monoglyceride+diglyceride+triglyceride content. In some embodiments of any of the aspects, the omega-3 fatty acids of the compositions described herein comprise no more than 10% monoglycerides+diglycerides, e.g., as a proportion of the total monoglyceride+diglyceride+triglyceride content. In some embodiments of any of the aspects, the omega-6 fatty acids of the compositions described herein comprise no more than 10% monoglycerides+diglycerides, e.g., as a proportion of the total monoglyceride+diglyceride+triglyceride content. In some embodiments of any of the aspects, the DHA of the compositions described herein comprise no more than 10% monoglycerides+diglycerides, e.g., as a proportion of the total monoglyceride+diglyceride+triglyceride content. In some embodiments of any of the aspects, the EPA of the compositions described herein comprise no more than 10% monoglycerides+diglycerides, e.g., as a proportion of the total monoglyceride+diglyceride+triglyceride content. In some embodiments of any of the aspects, the DHA and EPA, collectively, of the compositions described herein comprise no more than 10% monoglycerides+diglycerides, e.g., as a proportion of the total monoglyceride+diglyceride+triglyceride content. In some embodiments of any of the aspects, the ARA of the compositions described herein comprise no more than 10% monoglycerides+diglycerides, e.g., as a proportion of the total monoglyceride+diglyceride+triglyceride content. In some embodiments of any of the aspects, the total monoglyceride+diglyceride+triglyceride content of the compositions described herein comprises no more than 10% monoglycerides+diglycerides.

In some embodiments of any of the aspects, the omega-3 and/or omega-6 fatty acids of the compositions described herein comprise no diglycerides. In some embodiments of any of the aspects, the omega-3 fatty acids of the compositions described herein comprise no diglycerides. In some embodiments of any of the aspects, the omega-6 fatty acids of the compositions described herein comprise no diglycerides. In some embodiments of any of the aspects, the DHA of the compositions described herein comprise no diglycerides. In some embodiments of any of the aspects, the EPA of the compositions described herein comprise no diglycerides. In some embodiments of any of the aspects, the DHA and EPA, collectively, of the compositions described herein comprise no diglycerides. In some embodiments of any of the aspects, the ARA of the compositions described herein comprise no diglycerides. In some embodiments of any of the aspects, the compositions described herein comprise no diglycerides.

In one aspect of any of the embodiments, the composition and the components thereof are not distilled or re-esterified. In one aspect of any of the embodiments, the omega-6 fatty acids are not distilled or re-esterified. In one aspect of any of the embodiments, the omega-3 fatty acids are not distilled or re-esterified. In one aspect of any of the embodiments, the ARA is not distilled or re-esterified. In one aspect of any of the embodiments, the DHA is not distilled or re-esterified. In one aspect of any of the embodiments, the EPA is not distilled or re-esterified. In one aspect of any of the embodiments, the DHA and EPA, collectively, are not distilled or re-esterified.

In some embodiments, the fatty acid(s) are obtained by a transesterification of an oil described herein (e.g., plant, fish, algal, or microbial oil). In some embodiments, the fatty acid(s) are obtained by a transesterification of an oil described herein (e.g., plant, fish, algal, or microbial oil), and subsequent physio-chemical purification processes, including urea fractionation followed by molecular distillation. In some embodiments, the crude fatty acid(s) may also be subjected to a stripping process for decreasing the amount of environmental pollutants and/or cholesterol before the transesterification. In another embodiment, the fatty acid(s) are obtained by using supercritical CO₂ extraction or chromatography techniques, for example to up-concentrate specific fatty acids.

In some embodiments of any of the aspects, the composition described herein, or the components thereof (e.g. an omega-3 fatty acid and/or omega-6 fatty acid oil) have a low sterol level. As used herein, the term “sterol” refers to a compound comprising the following structure and optionally further comprising additional functional groups or ring system modifications.

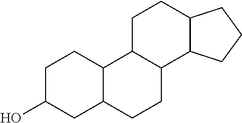 [SMILES: OC1CCC2C(CCC3C4CCCC4CCC23)C1]

In some embodiments, sterols can be steroids. Exemplary sterols include but are not limited to cholesterol, ergosterol, hydroxysteroid, phytosterols, steroids, and zoosterols.

In some embodiments of any of the aspects, the composition described herein, or the components thereof (e.g. an omega-3 fatty acid and/or omega-6 fatty acid oil) comprises no more than 10% (w/w) sterols. In some embodiments of any of the aspects, the composition described herein, or the components thereof (e.g. an omega-3 fatty acid and/or omega-6 fatty acid oil) comprises no more than 9% (w/w) sterols. In some embodiments of any of the aspects, the composition described herein, or the components thereof (e.g. an omega-3 fatty acid and/or omega-6 fatty acid oil) comprises no more than 8% (w/w) sterols. In some embodiments of any of the aspects, the composition described herein, or the components thereof (e.g. an omega-3 fatty acid and/or omega-6 fatty acid oil) comprises no more than 7% (w/w) sterols. In some embodiments of any of the aspects, the composition described herein, or the components thereof (e.g. an omega-3 fatty acid and/or omega-6 fatty acid oil) comprises no more than 6% (w/w) sterols. In some embodiments of any of the aspects, the composition described herein, or the components thereof (e.g. an omega-3 fatty acid and/or omega-6 fatty acid oil) comprises no more than 5% (w/w) sterols. In some embodiments of any of the aspects, the composition described herein, or the components thereof (e.g. an omega-3 fatty acid and/or omega-6 fatty acid oil) comprises no more than 4% (w/w) sterols. In some embodiments of any of the aspects, the composition described herein, or the components thereof (e.g. an omega-3 fatty acid and/or omega-6 fatty acid oil) comprises no more than 3% (w/w) sterols. In some embodiments of any of the aspects, the composition described herein, or the components thereof (e.g. an omega-3 fatty acid and/or omega-6 fatty acid oil) comprises no more than 2% (w/w) sterols. In some embodiments of any of the aspects, the composition described herein, or the components thereof (e.g. an omega-3 fatty acid and/or omega-6 fatty acid oil) comprises no more than 1.5% (w/w) sterols. In some embodiments of any of the aspects, the composition described herein, or the components thereof (e.g. an omega-3 fatty acid and/or omega-6 fatty acid oil) comprises no more than 1% (w/w) sterols.

In some embodiments of any of the aspects, the composition described herein, or the components thereof (e.g. an omega-3 fatty acid and/or omega-6 fatty acid oil) have a low phytosterol level. As used herein, “phytosterol” refers to sterols and stanols of plant origin, e.g., phytosteroids. Non-limiting examples of phytosterols can include β-sitosterol, campesterol, stigmasterol, sitostanol and campestanol.

In some embodiments of any of the aspects, the composition described herein, or the components thereof (e.g. an omega-3 fatty acid and/or omega-6 fatty acid oil) comprises no more than 120 mg/L phytosterols. In some embodiments of any of the aspects, the composition described herein, or the components thereof (e.g. an omega-3 fatty acid and/or omega-6 fatty acid oil) comprises no more than 110 mg/L phytosterols. In some embodiments of any of the aspects, the composition described herein, or the components thereof (e.g. an omega-3 fatty acid and/or omega-6 fatty acid oil) comprises no more than 100 mg/L phytosterols. In some embodiments of any of the aspects, the composition described herein, or the components thereof (e.g. an omega-3 fatty acid and/or omega-6 fatty acid oil) comprises no more than 80 mg/L phytosterols. In some embodiments of any of the aspects, the composition described herein, or the components thereof (e.g. an omega-3 fatty acid and/or omega-6 fatty acid oil) comprises no more than 70 mg/L phytosterols. In some embodiments of any of the aspects, the composition described herein, or the components thereof (e.g. an omega-3 fatty acid and/or omega-6 fatty acid oil) comprises no more than 60 mg/L phytosterols. In some embodiments of any of the aspects, the composition described herein, or the components thereof (e.g. an omega-3 fatty acid and/or omega-6 fatty acid oil) comprises no more than 50 mg/L phytosterols. In some embodiments of any of the aspects, the composition described herein, or the components thereof (e.g. an omega-3 fatty acid and/or omega-6 fatty acid oil) comprises no more than 45 mg/L phytosterols. In some embodiments of any of the aspects, the composition described herein, or the components thereof (e.g. an omega-3 fatty acid and/or omega-6 fatty acid oil) comprises no more than 40 mg/L phytosterols. In some embodiments of any of the aspects, the composition described herein, or the components thereof (e.g. an omega-3 fatty acid and/or omega-6 fatty acid oil) comprises no more than 35 mg/L phytosterols. In some embodiments of any of the aspects, the composition described herein, or the components thereof (e.g. an omega-3 fatty acid and/or omega-6 fatty acid oil) comprises no more than 30 mg/L phytosterols. In some embodiments of any of the aspects, the composition described herein, or the components thereof (e.g. an omega-3 fatty acid and/or omega-6 fatty acid oil) comprises no more than 25 mg/L phytosterols. In some embodiments of any of the aspects, the composition described herein, or the components thereof (e.g. an omega-3 fatty acid and/or omega-6 fatty acid oil) comprises no more than 20 mg/L phytosterols.

The level of phytosterols can be reduced or kept within a threshold described herein by various methods. For example, oils or compositions naturally low in phytosterols can be used. In some embodiments, a high phytosteroil oil can be combined with a low phytosterol oil in proportions that provide a final phytosterol content as described herein. In some embodiments, phytosterol can be removed from an oil, e.g., by purification.

In some embodiments of any of the aspects, the composition described herein, or the components thereof (e.g. an omega-3 fatty acid and/or omega-6 fatty acid oil) comprises no more than 20 mg/L stigmasterol. In some embodiments of any of the aspects, the composition described herein, or the components thereof (e.g. an omega-3 fatty acid and/or omega-6 fatty acid oil) comprises no more than 10 mg/L stigmasterol. In some embodiments of any of the aspects, the composition described herein, or the components thereof (e.g. an omega-3 fatty acid and/or omega-6 fatty acid oil) comprises no more than 5 mg/L stigmasterol. In some embodiments of any of the aspects, the composition described herein, or the components thereof (e.g. an omega-3 fatty acid and/or omega-6 fatty acid oil) comprises no more than 3 mg/L stigmasterol. In some embodiments of any of the aspects, the composition described herein, or the components thereof (e.g. an omega-3 fatty acid and/or omega-6 fatty acid oil) comprises no more than 2 mg/L stigmasterol. In some embodiments of any of the aspects, the composition described herein, or the components thereof (e.g. an omega-3 fatty acid and/or omega-6 fatty acid oil) comprises no more than 1 mg/L stigmasterol.

In some embodiments of any of the aspects, the composition described herein, or the components thereof (e.g. an omega-3 fatty acid and/or omega-6 fatty acid oil) comprises no more than 5% (w/w) stigmasterol. In some embodiments of any of the aspects, the composition described herein, or the components thereof (e.g. an omega-3 fatty acid and/or omega-6 fatty acid oil) comprises no more than 4% (w/w) stigmasterol. In some embodiments of any of the aspects, the composition described herein, or the components thereof (e.g. an omega-3 fatty acid and/or omega-6 fatty acid oil) comprises no more than 3% (w/w) stigmasterol. In some embodiments of any of the aspects, the composition described herein, or the components thereof (e.g. an omega-3 fatty acid and/or omega-6 fatty acid oil) comprises no more than 2% (w/w) stigmasterol. In some embodiments of any of the aspects, the composition described herein, or the components thereof (e.g. an omega-3 fatty acid and/or omega-6 fatty acid oil) comprises no more than 1% (w/w) stigmasterol. In some embodiments of any of the aspects, the composition described herein, or the components thereof (e.g. an omega-3 fatty acid and/or omega-6 fatty acid oil) comprises no more than 0.5% (w/w) stigmasterol.

In some embodiments of any of the aspects, the composition is formulated to comprise ARA at a dosage of 5-150 mg/kg/day. In some embodiments of any of the aspects, the composition is formulated to comprise DHA and/or EPA at a dosage of 10-200 mg/kg/day. In some embodiments of any of the aspects, the composition is formulated to comprise DHA and/or EPA at a dosage of 10 mg/kg/day to 3 g/kg/day. In some embodiments of any of the aspects, the composition is formulated to comprise ARA at a dosage of 5-150 mg/kg/day and DHA and/or EPA at a dosage of 10-200 mg/kg/day. In some embodiments of any of the aspects, the composition is formulated to comprise ARA at a dosage of 5-150 mg/kg/day and DHA and/or EPA at a dosage of 10 mg/kg/day-3 g/kg/day.

In some embodiments of any of the aspects, the composition is formulated to comprise ARA at a dosage of 10-120 mg/kg/day. In some embodiments of any of the aspects, the composition is formulated to comprise DHA and/or EPA at a dosage of 20-150 mg/kg/day. In some embodiments of any of the aspects, the composition is formulated to comprise ARA at a dosage of 10-120 mg/kg/day and/or DHA and/or EPA at a dosage of 20-150 mg/kg/day. In some embodiments of any of the aspects, the composition is formulated to comprise ARA at a dosage of 10-120 mg/kg/day and DHA and/or EPA at a dosage of 20-150 mg/kg/day.

In some embodiments of any of the aspects, the composition is formulated to comprise ARA at a dosage of 20-60 mg/kg/day. In some embodiments of any of the aspects, the composition is formulated to comprise DHA and/or at a dosage of 40-100 mg/kg/day. In some embodiments of any of the aspects, the composition is formulated to comprise ARA at a dosage of 20-60 mg/kg/day and/or DHA and/or ePA at a dosage of 40-100 mg/kg/day. In some embodiments of any of the aspects, the composition is formulated to comprise ARA at a dosage of 20-60 mg/kg/day and DHA and/or EPA at a dosage of 40-100 mg/kg/day.

In some embodiments of any of the aspects, the composition is formulated to comprise ARA at a dosage of 20-200 mg/kg/day. In some embodiments of any of the aspects, the composition is formulated to comprise DHA and/or at a dosage of 40-400 mg/kg/day. In some embodiments of any of the aspects, the composition is formulated to comprise ARA at a dosage of 20-200 mg/kg/day and/or DHA and/or ePA at a dosage of 40-400 mg/kg/day. In some embodiments of any of the aspects, the composition is formulated to comprise ARA at a dosage of 20-200 mg/kg/day and DHA and/or EPA at a dosage of 40-400 mg/kg/day.

In the foregoing doses, “kg” refers to the mass of the subject being administered the composition.

In some embodiments of any of the aspects, the composition is a lipid emulsion, e.g., a lipid in water emulsion.

In some embodiments of any of the aspects, the composition (e.g., the emulsion) is at least 5% oil in water. In some embodiments of any of the aspects, the composition (e.g., the emulsion) is at least 10% oil in water. In some embodiments of any of the aspects, the composition (e.g., the emulsion) is at least 15% oil in water. In some embodiments of any of the aspects, the composition (e.g., the emulsion) is at least 20% oil in water. In some embodiments of any of the aspects, the composition (e.g., the emulsion) is at least 25% oil in water. In some embodiments of any of the aspects, the composition (e.g., the emulsion) is at least 30% oil in water. In some embodiments of any of the aspects, the composition (e.g., the emulsion) is at least 35% oil in water. In some embodiments of any of the aspects, the composition (e.g., the emulsion) is at least 40% oil in water.

In some embodiments of any of the aspects, the composition (e.g., the emulsion) is about 10% to about 50% oil in water. In some embodiments of any of the aspects, the composition (e.g., the emulsion) is about 10% to about 40% oil in water. In some embodiments of any of the aspects, the composition (e.g., the emulsion) is about 10% to about 30% oil in water. In some embodiments of any of the aspects, the composition (e.g., the emulsion) is about 20% to about 50% oil in water. In some embodiments of any of the aspects, the composition (e.g., the emulsion) is about 20% to about 40% oil in water. In some embodiments of any of the aspects, the composition (e.g., the emulsion) is about 25% to about 35% oil in water.

In some embodiments of any of the aspects, the composition (e.g., the emulsion) is 10% to 50% oil in water. In some embodiments of any of the aspects, the composition (e.g., the emulsion) is 10% to 40% oil in water. In some embodiments of any of the aspects, the composition (e.g., the emulsion) is 10% to 30% oil in water. In some embodiments of any of the aspects, the composition (e.g., the emulsion) is 20% to 50% oil in water. In some embodiments of any of the aspects, the composition (e.g., the emulsion) is 20% to 40% oil in water. In some embodiments of any of the aspects, the composition (e.g., the emulsion) is 25% to 35% oil in water.

In some embodiments of any of the aspects, the omega-6 fatty acids (e.g., ARA) and omega-3 fatty acids (e.g., DHA and/or EPA) described herein comprise from about 5% w/v to about 50% w/v of total fats in the total volume of the composition. In some embodiments of any of the aspects, the omega-6 fatty acids (e.g., ARA) and omega-3 fatty acids (e.g., DHA and/or EPA) described herein comprise from about 15% w/v to about 25% w/v of total fats in the total volume of the composition. In some embodiments of any of the aspects, the omega-6 fatty acids (e.g., ARA) and omega-3 fatty acids (e.g., DHA and/or EPA) described herein comprise from about 5% w/v to about 50% w/v of total fats in the total volume of the composition. In some embodiments of any of the aspects, the omega-6 fatty acids (e.g., ARA) and omega-3 fatty acids (e.g., DHA and/or EPA) described herein comprise from about 20% w/v of total fats in the total volume of the composition. In some embodiments of any of the aspects, the omega-6 fatty acids (e.g., ARA) and omega-3 fatty acids (e.g., DHA and/or EPA) described herein comprise from 5% w/v to 50% w/v of total fats in the total volume of the composition. In some embodiments of any of the aspects, the omega-6 fatty acids (e.g., ARA) and omega-3 fatty acids (e.g., DHA and/or EPA) described herein comprise from 15% w/v to 25% w/v of total fats in the total volume of the composition. In some embodiments of any of the aspects, the omega-6 fatty acids (e.g., ARA) and omega-3 fatty acids (e.g., DHA and/or EPA) described herein comprise from 5% w/v to 50% w/v of total fats in the total volume of the composition. In some embodiments of any of the aspects, the omega-6 fatty acids (e.g., ARA) and omega-3 fatty acids (e.g., DHA and/or EPA) described herein comprise 20% w/v of total fats in the total volume of the composition.

The composition, e.g, the emulsion, described herein can be administered, e.g., orally, parenterally, or intravenously. In some embodiments of any of the aspects, the composition (e.g., the emulsion) described herein is formulated for oral administration. In some embodiments of any of the aspects, the composition (e.g., the emulsion) described herein is formulated for parenteral and/or intravenous administration.

In some embodiments of any of the aspects, the compositions described herein can be prepared by first emulsifying each component as a separate emulsion and then mixing or combining those emulsions together, e.g., by emulsifying an omega-3 fatty acid oil source (e.g., a fish or algal oil), emulsifying an omega-6 fatty acid oil source (e.g., a plant or fungal oil), and then mixing or combining those two emulsifications together to yield the final combined emulsion in the aforementioned combinations.

In some embodiments of any of the aspects, each emulsion can be individually formulated in the following manner via high-pressure homogenization: first a lipid dispersion is created using an egg phospholipid emulsifier that is added to heated, (75-90° C.), USP-grade sterile water for injection (SWFI), under high-speed shear mixing conditions. The temperature is allowed to decrease to 40-45° C. Sodium oleate is then added and shear mixing continued at 3900-4000 RPM for 40 minutes. Heated SWFI is then serially added to maintain the temperature at 40-45° C. Glycerin is added under continuous shear mixing. This results in a dispersion comprised of 12% egg phospholipid, 25% glycerin, and 0.3% sodium oleate. The crude dispersion is then transferred to a homogenizer and homogenized at 9000 psi at 40-45° C. for 20 cycles, filtered through a 0.45 μm membrane, and pH is adjusted to 10.4 with 0.5N sodium hydroxide (NaOH). All steps are performed under a nitrogen atmosphere. To compound the emulsions, the oil (e.g., omega-3 or omega-6 oil) is added to the dispersion agent in a thin stream under continuous shear mixing conditions at 3500-4500 RPM for 40-45 minutes, maintaining a 40-45° C. temperature. The resulting crude emulsions are transferred to the homogenizer and homogenized at 5000 psi and 40-45° C. for no less than 9 cycles of the emulsion. The pH of the emulsions are buffered to >8.8 using 0.1N NaOH. All steps of the compounding process are performed under a nitrogen atmosphere. The finished emulsions are aliquoted into glass serum vials and headspaces are flooded with nitrogen gas before sealing. All vials were heat sterilized. This allows optimal homogenization of each oil type to improve compliance with Chapter <729> of the United States Pharmacopoeia (USP) requirements that parenteral fat emulsions have a mean fat globule size <500 nm and a percent of fat globules >5 μm (PFAT5)≤0.05%.

In some embodiments of any of the aspects, the emulsions described herein can be a mixture of two or more emulsions. This approach can have the advantage of allowing additional components to be readily added to the mixture after the intial emulsification of the lipids. By way of non-limiting example, a 50:50 blend of an omega-3 oil and an omega-6 oil can be further supplemented with, e.g., additional oil emulsion, trigylcerides, DHA, EPA, ARA, or other lipids as needed, for a patient specific condition at the time of administration as opposed to the time of manufacture. This allows more flexibility for the prescriber to titrate the blend of oils for patient specific conditions and decreases the need for practitioners to maintain multiple emulsion combinations.

In some embodiments of any of the aspects, the compositions described herein can be prepared by first mixing the components together and then preparing an emulsification of the mixture, e.g., by mixing an omega-3 oil and an omega-6 oil, and then emulsifying the mixture. In some embodiments of any of the aspects, the emulsions described herein can be an emulsification of a mixture of lipid preparations.

To prepare the emulsions in accordance with the present invention, one or more emulsifying agents can be mixed with, e.g., the source of omega-3 oil, omega-6 oil, ARA, EPA, or DHA. Emulsifying agents for this purpose are generally phospholipids of natural, synthetic or semi-synthetic origin. A variety of suitable emulsifying agents are known in the art. Examples of suitable emulsifying agents include, but are not limited to, egg phosphatidylcholine, egg lecithin, L-α-dipalmitoyl phosphatidylcholine (DPPC), DL-α-dipalmitoyl phosphatidylethanolamine (DPPE), and dioleoyl phosphatidylcholine (DOPC). In accordance with the present invention, the total concentration of diglycerides and monoglyceride as well as free fatty acids in the emulsifier should be low in order to minimize the contribution to the total oil concentration of the emulsion. In one embodiment of the present invention, the total concentration of triglycerides as well as free fatty acids in the emulsifier is less than about 3.5%. In some embodiments of any of the aspects, lecithin is used as the emulsifying agent in the lipid emulsions. Alternatively, egg lecithin can be used as the emulsifying agent. Egg lecithin containing 80-85% phosphatidyl choline and less than about 3.5% of fat can also be used as an emulsifying agent. One skilled in the art will appreciate that other components may be present in the egg lecithin without adversely affecting the emulsifying properties. For example, the egg lecithin may contain one or more of phosphatidyl ethanolamine, lysophosphatidyl choline, lysophosphatidyl ethanolamine, sphingomeylin and other natural components.

In some embodiments of any of the aspects, an emulsion as described herein comprises between about 0.5% and about 5% (w/v) emulsifying agent. In some embodiments of any of the aspects, an emulsion as described herein comprises between about 0.6% and about 2% (w/v) emulsifying agent. In some embodiments of any of the aspects, an emulsion as described herein comprises between about 0.8% and about 1.8% (w/v) emulsifying agent. In some embodiments of any of the aspects, an emulsion as described herein comprises between about 1.0% and about 1.5% (w/v) emulsifying agent. In some embodiments of any of the aspects, an emulsion as described herein comprises about 1.2% (w/v) emulsifying agent.

The ratio of lecithin to oil in the emulsion is important in determining the size of the oil globules formed within the emulsion. In some embodiments of any of the aspects, the ratio of lecithin to oil is between about 1:4 and about 1:20. In some embodiments of any of the aspects, the ratio is between about 1:4 and about 1:18. In some embodiments of any of the aspects, the ratio is between about 1:4 and about 1:15. In some embodiments of any of the aspects, the ratio is between about 1:4 and about 1:10.

The composition, e.g., lipid emulsion, in accordance with the present invention can further comprise additional components such as, antioxidants, chelating agents, osmolality modifiers, buffers, neutralization agents and the like that improve the stability, uniformity and/or other properties of the composition (e.g., the emulsion). Suitable antioxidants that can be added to the lipid emulsions include, but are not limited to, alpha-tocopherol (vitamin E) and tocotrienols. As is known in the art, tocotrienols are a natural blend of tocotrienols and vitamin E extract concentrated from rice bran oil distillate. Tocotrienols have a similar structure to vitamin E and contain three double bonds in the carbon side chain of the molecule. In some embodiments of any of the aspects, the concentration of antioxidant added to the composition (e.g., the emulsion) is typically between about 0.002 and about 1.0% (w/v). In some embodiments of any of the aspects, the concentration of antioxidant used in the composition (e.g., the emulsion) is between about 0.02% and about 0.5% (w/v). In some embodiments of any of the aspects, tocotrienols are added to the composition (e.g., the emulsion) as an antioxidant. In some embodiments of any of the aspects, about 0.5% (w/v) tocotrienols are added to the composition (e.g., the emulsion). In some embodiments of any of the aspects, vitamin E is added to the composition (e.g., the emulsion) as an antioxidant. In some embodiments of any of the aspects, about 0.02% (w/v) vitamin E is added to the composition (e.g., the emulsion).

In some embodiments of any of the aspects, alpha-tocopherol is present in the emulsion at a level of at least 50 mg/L. In some embodiments of any of the aspects, alpha-tocopherol is present in the emulsion at a level of at least 75 mg/L. In some embodiments of any of the aspects, alpha-tocopherol is present in the emulsion at a level of at least 100 mg/L. In some embodiments of any of the aspects, alpha-tocopherol is present in the emulsion at a level of at least 120 mg/L. In some embodiments of any of the aspects, alpha-tocopherol is present in the emulsion at a level of at least 150 mg/L. In some embodiments of any of the aspects, alpha-tocopherol is present in the emulsion at a level of at least 200 mg/L.

The composition (e.g., the emulsion) can further comprise a chelating agent to improve the stability of the composition (e.g., the emulsion) and reduce the formation of oxidized fatty acids. Suitable chelating agents are known in the art and are those that are generally recognized as safe (GRAS) compounds. Examples include, but are not limited to, EDTA. In some embodiments of any of the aspects, the composition (e.g., the emulsion) comprises EDTA. In some embodiments of any of the aspects, the composition (e.g., the emulsion) comprises concentrations of EDTA between about 1×10⁻⁶ M and 5×10⁻⁵ M.

An osmolality modifier can also be incorporated into the composition (e.g., the emulsion) to adjust the osmolality of the composition (e.g., the emulsion) to a value suitable for parenteral administration. Amounts and types of osmolality modifiers for use in parenteral emulsions are well-known in the art. An example of a suitable osmolality modifier is glycerol. The concentration of osmolality modifier typically ranges from about 2% to about 5% (w/v). In some embodiments of any of the aspects, the amount of osmolality modifier added to the composition (e.g., the emulsion) is between about 2% and about 4%. In some embodiments of any of the aspects, the amount of osmolality modifier added to the composition (e.g., the emulsion) is between about 2% and about 3%. In some embodiments of any of the aspects, about 2.25% (w/v) glycerol is added to the composition (e.g., the emulsion) as an osmolality modifier. In some embodiments of any of the aspects, the final product is isotonic so as to allow infusion of the composition (e.g., the emulsion) through either a central or peripheral venous catheter.

The pH of the composition (e.g., the emulsion) can be adjusted through the use of buffers or neutralization agents. Emulsions with pH values close to physiological pH or above have been shown to be less prone to fatty acid peroxidation. One skilled in the art will appreciate that the pH of the composition (e.g., the emulsion) can be adjusted through the use of an appropriate base that neutralizes the negative charge on the fatty acids, through the use of an appropriate buffer, or a combination thereof. A variety of bases and buffers are suitable for use with the compositions (e.g., the emulsions) of the present invention. One skilled in the art will appreciate that the addition of buffer to the composition (e.g., the emulsion) will affect not only on the final pH, but also the ionic strength of the composition (e.g., the emulsion). High ionic strengths may negatively impact the zeta potential of the composition (e.g., the emulsion) (i.e. the surface charge of the oil globules) and are, therefore, not desirable. Selection of an appropriate buffer strength to provide a suitable pH is considered to be within the ordinary skills of a worker in the art. In some embodiments of any of the aspects, the pH of the composition (e.g., the emulsion) is adjusted using sodium hydroxide. In some embodiments of any of the aspects, the pH is adjusted with a buffer. In some embodiments of any of the aspects, the buffer is a phosphate buffer. In some embodiments of any of the aspects, both sodium hydroxide and a phosphate buffer are added to the composition (e.g., the emulsion). In some embodiments of any of the aspects, the final pH of the composition (e.g., the emulsion) is between about 6.0 and about 9.0. In some embodiments of any of the aspects, the pH of the composition (e.g., the emulsion) is between about 7.0 and about 8.5. In some embodiments of any of the aspects, the pH of emulsion is between about 7.0 and about 8.0.

In some embodiments of any of the aspects, the lipid emulsion can further comprise components for adjusting the stability of the composition (e.g., the emulsion), for example, amino acids or carbohydrates, such as fructose, glucose, or dextrose. The lipid emulsion can also be formulated to include nutrients such as glucose or dextrose, amino acids, vitamins, or other parenteral nutritional supplements. The formulation of the lipid emulsion to incorporate a therapeutic agent is also considered to be within the scope of the present invention. A “therapeutic agent” as used herein refers to a physiologically or pharmacologically active substance that produces a localized or systemic effect or effects in animals and refers generally to drugs, nutritional supplements, vitamins, minerals, enzymes, hormones, proteins, polypeptides, antigens and other therapeutically or diagnostically useful compounds.

In some embodiments of any of the aspects, the composition (e.g, the emulsion) can further comprise an additive of one or more additional fatty acids or a mixture thereof. In some embodiments of any of the aspects, the methods described herein can further comprise administering an additive of one or more additional fatty acids or a mixture thereof. The additive can comprise one or more fatty acids which are therapeutic for a disease, e.g., a disease which the subject is in need of treatment for. For example, DHA can be therapeutic for patients with cystic fibrosis, and an additive for a subject with cystic fibrosis can comprise DHA, e.g., beyond what is necessary for nutritional balance. In some embodiments of any of the aspects, the additive comprises fatty acids in a ratio or blend which is therapeutic. In some embodiments of any of the aspects, the additive is provided at a dose of nanograms to grams/kg/day. In some embodiments of any of the aspects, the additive is provided at a dose of 1 nanogram to 10 grams/kg/day. In some embodiments of any of the aspects, the additive is provided at a dose of 1 nanogram to 100 grams/kg/day. In some embodiments of any of the aspects, the additive is provided at a dose of 1 nanogram to 1000 grams/kg/day.

In some embodiments of any of the aspects, the composition further comprises one or more of Medium chain triglycerides (MCTs); egg lecithin; sunflower seed oil; sunflower lecithin; an emulsifier obtained from sunflower seed; and krill oil/krill lecithin. Sunflower lecithin is an emulsifier noted for a higher phosphotidyl choline content and is available commerically, e.g, from Lipoid. Krill oil or krill lecithin is self-emulsifying due to high phospholipid content.

As used herein, “medium-chain triglycerides” or “MCT” refer to triglycerides having fatty acids of 6-12 carbon atom chains. A triglyceride (“TG”) (also known as triacylglycerol or triacylglyceride) is a glyceride in which the glycerol is esterified with three fatty acids. MCTs can comprise fatty acids selected from, e.g., caprylic acid, capric acid, lauric acid, and caproic acid. MCTs can be derived from a plant such as a fruit or vegetable, for example, a plurality of plants. The description of the MCT for use in this disclosure can, for example, meet the requirements of EP monograph 0868, entitled “Triglycerides, Medium Chain” (Triglycerida saturate media) (EP 0868, 2008); which is incorporated by reference herein in its entirety. In some embodiments of any of the aspects, the MCT comprises omega-9 fatty acids and/or saturated fatty acids.

In some embodiments of any of the aspects, the compositions (e.g., emulsions) described herein can further comprise alpha-tocopherol. As used herein, “alpha-tocopherol” refers to the form of vitamin E having the structure of Formula I, in any of its stereoisomers.

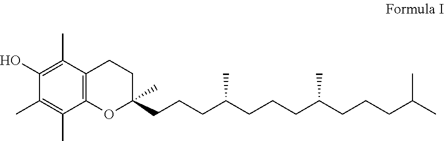 [SMILES: Cc1c(C)c2c(c(C)c1O)CC[C@@](C)(CCC[C@H](C)CCC[C@H](C)CCCC(C)C)O2]

In some embodiments of any of the aspects, alpha-tocopherol is present in the composition (e.g., the emulsion) at a level of at least 50 mg/L. In some embodiments of any of the aspects, alpha-tocopherol is present in the composition (e.g., the emulsion) at a level of at least 75 mg/L. In some embodiments of any of the aspects, alpha-tocopherol is present in the composition (e.g., the emulsion) at a level of at least 100 mg/L. In some embodiments of any of the aspects, alpha-tocopherol is present in the composition (e.g., the emulsion) at a level of at least 120 mg/L. In some embodiments of any of the aspects, alpha-tocopherol is present in the composition (e.g., the emulsion) at a level of at least 150 mg/L. In some embodiments of any of the aspects, alpha-tocopherol is present in the composition (e.g., the emulsion) at a level of at least 200 mg/L.

In some embodiments of any of the aspects, the composition (e.g., the emulsion) comprises alpha-tocopherol and other forms of vitamin E (e.g., beta tocopherol, gamma tocopherol, delta tocopherol, alpha tocotrienol, beta tocotrienol, gamma tocotrienol, and/or delta tocotrienol) at a ratio of at least 2:1 by weight. In some embodiments of any of the aspects, the composition (e.g., the emulsion) comprises alpha-tocopherol and other forms of vitamin E at a ratio of at least 3:1 by weight. In some embodiments of any of the aspects, the composition (e.g., the emulsion) comprises alpha-tocopherol and other forms of vitamin E at a ratio of at least 5:1 by weight. In some embodiments of any of the aspects, the composition (e.g., the emulsion) comprises alpha-tocopherol and other forms of vitamin E at a ratio of at least 10:1 by weight. In some embodiments of any of the aspects, the composition (e.g., the emulsion) does not comprise forms of vitamin E other than alpha-tocopherol.

In some embodiments of any of the aspects, a composition (e.g., an emulsion) as described herein can comprise one or more omega-6 fatty acids comprising at least Arachidonic acid (ARA) and one or more omega-3 fatty acids comprising at least Docosahexaenoic acid (DHA) and/or EPA. In some embodiments of any of the aspects, a composition (e.g., an emulsion) as described herein can consist essentially of one or more omega-6 fatty acids comprising at least Arachidonic acid (ARA) and one or more omega-3 fatty acids comprising at least Docosahexaenoic acid (DHA) and/or EPA. In some embodiments of any of the aspects, a composition (e.g., an emulsion) as described herein can consist of one or more omega-6 fatty acids comprising at least Arachidonic acid (ARA) and one or more omega-3 fatty acids comprising at least Docosahexaenoic acid (DHA) and/or EPA.

In some embodiments of any of the aspects, a composition (e.g., an emulsion) as described herein can comprise one or more omega-6 fatty acids comprising at least Arachidonic acid (ARA) and one or more omega-3 fatty acids comprising at least Docosahexaenoic acid (DHA). In some embodiments of any of the aspects, a composition (e.g., an emulsion) as described herein can consist essentially of one or more omega-6 fatty acids comprising at least Arachidonic acid (ARA) and one or more omega-3 fatty acids comprising at least Docosahexaenoic acid (DHA). In some embodiments of any of the aspects, a composition (e.g., an emulsion) as described herein can consist of one or more omega-6 fatty acids comprising at least Arachidonic acid (ARA) and one or more omega-3 fatty acids comprising at least Docosahexaenoic acid (DHA).

In some embodiments of any of the aspects, a composition (e.g., an emulsion) as described herein can comprise one or more omega-6 fatty acids comprising at least Arachidonic acid (ARA) and one or more omega-3 fatty acids comprising at least EPA. In some embodiments of any of the aspects, a composition (e.g., an emulsion) as described herein can consist essentially of one or more omega-6 fatty acids comprising at least Arachidonic acid (ARA) and one or more omega-3 fatty acids comprising at least EPA. In some embodiments of any of the aspects, a composition (e.g., an emulsion) as described herein can consist of one or more omega-6 fatty acids comprising at least Arachidonic acid (ARA) and one or more omega-3 fatty acids comprising at least EPA.

In some embodiments of any of the aspects, the omega-6 fatty acids comprise ARA. In some embodiments of any of the aspects, the omega-6 fatty acids consist essentially of ARA. In some embodiments of any of the aspects, the omega-6 fatty acids consist of ARA. In some embodiments of any of the aspects, the omega-3 fatty acids comprise DHA and EPA. In some embodiments of any of the aspects, the omega-3 fatty acids consist essentially of DHA and EPA. In some embodiments of any of the aspects, the omega-3 fatty acids consist of DHA and EPA. In some embodiments of any of the aspects, the omega-3 fatty acids comprise DHA. In some embodiments of any of the aspects, the omega-3 fatty acids consist essentially of DHA. In some embodiments of any of the aspects, the omega-3 fatty acids consist of DHA. In some embodiments of any of the aspects, the omega-3 fatty acids comprise EPA. In some embodiments of any of the aspects, the omega-3 fatty acids consist essentially of EPA. In some embodiments of any of the aspects, the omega-3 fatty acids consist of EPA

In some embodiments of any of the aspects, a composition (e.g., an emulsion) as described herein can comprise a) one or more omega-6 fatty acids comprising at least Arachidonic acid (ARA), b) one or more omega-3 fatty acids comprising at least Docosahexaenoic acid (DHA) and/or EPA, and c) an emulsifier (e.g., phospholipids and/or egg phospholipids). In some embodiments of any of the aspects, a composition (e.g., an emulsion) as described herein can consist essentially of a) one or more omega-6 fatty acids comprising at least Arachidonic acid (ARA), b) one or more omega-3 fatty acids comprising at least Docosahexaenoic acid (DHA) and/or EPA, and c) an emulsifier (e.g., phospholipids and/or egg phospholipids). In some embodiments of any of the aspects, a composition (e.g., an emulsion) as described herein can consist of a) one or more omega-6 fatty acids comprising at least Arachidonic acid (ARA), b) one or more omega-3 fatty acids comprising at least Docosahexaenoic acid (DHA) and/or EPA and c) an emulsifier (e.g., phospholipids and/or egg phospholipids).

In some embodiments of any of the aspects, a composition (e.g., an emulsion) as described herein can comprise a) one or more omega-6 fatty acids comprising at least Arachidonic acid (ARA), b) one or more omega-3 fatty acids comprising at least Docosahexaenoic acid (DHA) and/or EPA, and c) one or more of an emulsifier (e.g., phospholipids and/or egg phospholipids), glycerin, and sodium oleate. In some embodiments of any of the aspects, a composition (e.g., an emulsion) as described herein can consist essentially of a) one or more omega-6 fatty acids comprising at least Arachidonic acid (ARA), b) one or more omega-3 fatty acids comprising at least Docosahexaenoic acid (DHA) and/or EPA, and c) one or more of an emulsifier (e.g. phospholipids and/or egg phospholids), glycerin, and sodium oleate. In some embodiments of any of the aspects, a composition (e.g., an emulsion) as described herein can consist of a) one or more omega-6 fatty acids comprising at least Arachidonic acid (ARA), b) one or more omega-3 fatty acids comprising at least Docosahexaenoic acid (DHA) and/or EPA and c) one or more of an emulsifier (e.g. phospholipids and/or egg phospholids), glycerin, and sodium oleate.

In some embodiments of any of the aspects, a composition (e.g., an emulsion) as described herein can comprise a) one or more omega-6 fatty acids comprising at least Arachidonic acid (ARA), b) one or more omega-3 fatty acids comprising at least Docosahexaenoic acid (DHA) and/or EPA, and c) water. In some embodiments of any of the aspects, a composition (e.g., an emulsion) as described herein can consist essentially of a) one or more omega-6 fatty acids comprising at least Arachidonic acid (ARA), b) one or more omega-3 fatty acids comprising at least Docosahexaenoic acid (DHA) and/or EPA, and c) water. In some embodiments of any of the aspects, a composition (e.g., an emulsion) as described herein can consist of a) one or more omega-6 fatty acids comprising at least Arachidonic acid (ARA), b) one or more omega-3 fatty acids comprising at least Docosahexaenoic acid (DHA) and/or EPA and c) water.

In some embodiments of any of the aspects, a composition (e.g., an emulsion) as described herein can comprise a) one or more omega-6 fatty acids comprising at least Arachidonic acid (ARA), b) one or more omega-3 fatty acids comprising at least Docosahexaenoic acid (DHA) and/or EPA, c) water, and d) one or more of an emulsifier (e.g., phospholipids and/or egg phospholipids), glycerin, and sodium oleate. In some embodiments of any of the aspects, a composition (e.g., an emulsion) as described herein can consist essentially of a) one or more omega-6 fatty acids comprising at least Arachidonic acid (ARA), b) one or more omega-3 fatty acids comprising at least Docosahexaenoic acid (DHA) and/or EPA, c) water, and d) one or more of an emulsifier (e.g. phospholipids and/or egg phospholids), glycerin, and sodium oleate. In some embodiments of any of the aspects, a composition (e.g., an emulsion) as described herein can consist of a) one or more omega-6 fatty acids comprising at least Arachidonic acid (ARA), b) one or more omega-3 fatty acids comprising at least Docosahexaenoic acid (DHA) and/or EPA c) water, and d) one or more of an emulsifier (e.g. phospholipids and/or egg phospholids), glycerin, and sodium oleate.

In some embodiments of any of the aspects, the compositions (e.g., emulsions) described herein can further comprise one or more of an emulsifier (e.g. phospholipids and/or egg phospholids), glycerin, and sodium oleate. In some embodiments of any of the asepcts, the composition (e.g., the emulsion) described herein can comprise 0.5-2.5% egg phospholipid, 0.5-5.0% glycerin, and 0.005-0.1% sodium oleate.

In one aspect, described herein is a kit comprising a composition as described herein, e.g., a composition (e.g., an emulsion) as described herein. A kit is any manufacture (e.g., a package or container) comprising at least one reagent, e.g., the composition (e.g., the emulsion), being promoted, distributed, or sold as a unit for performing the methods described herein. The kits described herein can optionally comprise additional components useful for performing the methods described herein, e.g., needles, tubing, etc useful for administration by the desired route. By way of example, the kit can comprise fluids (e.g., buffers) suitable for use with the compositions (e.g., emulsions) described herein, an instructional material which describes performance of a method as described herein, and the like. A kit can further comprise devices and/or reagents for delivery of the composition as described herein. Additionally, the kit may comprise an instruction leaflet and/or may provide information as to dosages, administration frequency, etc.

The kits of the invention comprise one or more packages or containers containing the composition (e.g., the emulsion) in combination with a set of instructions, generally written instructions, relating to the use and dosage of the composition (e.g., the emulsion). The kits may further comprise additional containers containing one or more nutrients or therapeutic or diagnostic compounds that may be added to the composition (e.g., the emulsion) prior to administration. The packages containing the composition (e.g., the emulsion) may be in the form of unit doses or pharmacy bulk packages. The doses may be packaged in a format such that each dose is associated, for example, with a day of the week. There may also be associated with the kit a notice in the form prescribed by a governmental agency regulating the manufacture, use or sale of biological products, which notice reflects approval by the agency of manufacture, use or sale for human or animal administration.

Container design is also an important factor when manufacturing fat emulsions. For example, if the composition (e.g., the emulsion) is packaged in glass, the container can be filled with nitrogen before the actual emulsion is added. After addition of the composition (e.g., the emulsion), the glass container can be filled again with nitrogen to remove dead space when the cap is affixed. Such nitrogen filling prevents peroxide formation. If the product is packaged in plastic, a DEHP free container that is gas impermeable can be used. The container can also have the appropriate overwrap to minimize peroxide formation in the lipids as well as leaching of the plasticizer from the container into the product itself. In addition, if plastic is used, a desiccant can be included in with the bag as well as an indicator that notes if there is a air leak in the overwrap. In some embodiments of any of the aspects, the container can be latex-free.

In some embodiments, the kit comprises a bag, bottle, or syringe comprising a composition described herein. In some embodiments of any of the aspects, the composition is formulated for parenteral administration and is one or more of: sterile, free of visible particulate matter, and endotoxin-free. In some embodiments of any of the aspects, the composition is formulated for oral administration. In some embodiments of any of the aspects, a kit comprises a baby formula in a mixture or admixture with a composition described herein, e.g., for use by parents and caregivers. In some embodiments of any of the aspects, a kit comprises a baby formula in a mixture or admixture with a composition described herein, e.g., for use in a non-therapetic method.

In some embodiments of any of the aspects, the kit comprises a container comprising a composition described herein and a dosage device. The dosage device permits the user to measure or dispense a pre-determined dose. Exemplary, but non-limiting dosage devices can include a measuring cup, a syringe, a container comprising only a single dose, a spoon, and the like. In some embodiments of any of the aspects, the dosage device is sized to provide a dose of no more than 5 g fatty acids/kg/day. In some embodiments of any of the aspects, the dosage device is sized to provide a dose of no more than 4 g fatty acids/kg/day. In some embodiments of any of the aspects, the dosage device is sized to provide a dose of no more than 3 g fatty acids/kg/day. In some embodiments of any of the aspects, the dosage device is sized to provide a dose of no more than 5 cc/day. In some embodiments of any of the aspects, the dosage device is sized to provide a dose of no more than 4 cc/day. In some embodiments of any of the aspects, the dosage device is sized to provide a dose of no more than 3 cc/day.

In one aspect of any of the embodiments, described herein is a method comprising administering a formulation (e.g., a composition or emulsion) as described herein to a subject in need thereof. A subject in need of a formulation as described herein can be a subject in need of supplemental nutrition (either orally or parenterally), a subject in need of parenteral nutrition, or a subject in need of total parenteral nutrition. One of skill in the art can readily identify subjects who are in need of a formulation as described herein, e.g., subjects receiving parenteral nutrition include those who are evidencing symptoms are accepted as indicators that traditional PN or TPN should be reduced or stopped due to risk of side effects (including liver disease). Such subjects and their symptoms are described in detail, e.g., in Guidelines for the Provision and Assessment of Nutrition Support Therapy in the Adult Critically Ill Patient: Society of Critical Care Medicine (SCCM) and American Society for Parenteral and Enteral Nutrition (A.S.P.E.N.), Taylor et al., which is incorporated by reference herein in its entirety (available on the world wide web at journals.lww.com/ccmjournal/Fulltext/2016/02000/Guidelines_for_the_Provision_and_Assessment_of.20.aspx)

The compositions described herein can be utilized in at least two ways for two different goals. In the first approach, the compositions provided herein are administered parenterally as nutrition (e.g., by total parenteral nutrition), in order to provide fatty acids for growth and development. This approach is particularly applicable for premature infants, and most particularly for infants born at 34 weeks of pregnancy or earlier, where the digestive system is not yet developed sufficiently to permit fully enteral nutrition. In a second approach, the compositions provided herein are administered as a supplement (e.g., via a central or peripheral intravenous catheter, subcutaneous injection, or enteral administration), to provide additional fatty acids needs for growth and development. In this second approach, the compositions can provide more DHA/ARA than is available in, or is able to be digested from, other lipid emulsions or enteral human milk or infant formula.

In one aspect of any of the embodiments, described herein is a method of providing nutrition to a subject and/or promoting neurodevelopment in a subject, the method comprising administering the composition described herein to the subject. In some embodiments of any of the aspects, the neurodevelopment is neurodevelopment in the brain and/or eyes.

In some embodiments of any of the aspects, the administration treats, prevents, or reduces the risk of one or more conditions selected from the group consisting of: retinopathy; bronchopulmonary dysplasia; and perinatal sepsis. In one aspect of any of the embodiments, described herein is a method of treating, preventing, or reducing the risk of one or more conditions selected from the group consisting of: retinopathy; bronchopulmonary dysplasia; and perinatal sepsis, the method comprising administering the composition described herein to the subject. In some embodiments any of the aspects, the subject treated according to the methods described herein has, is diagnosed as having, or is at risk of retinopathy, bronchopulmonary dysplasia, or perinatal sepsis.

In some embodiments of any of the aspects, the patient administered a composition (e.g., an emulsion) described herein can be a subject in need of treatment for a condition selected from the group consisting of: hepatic steatosis; intestinal failure; parenteral nutrition-associated liver disease (PNALD); sepsis; cystic fibrosis; sickle cell anemia; pancreatitis; inflammatory bowel disease; Crohn's disease; biliary atresia; primary sclerosis cholangitis; an inflammatory infection; an inflammatory condition; systemic inflammatory response syndrome (SIRS); hypertriglyceridemia; severe hypertriglyceridemia; severe hepatic steatosis; retinopathy of prematurity; acute tubular necrosis; IgA nephropathies; ischemia-reperfusion injury; traumatic brain injury; multi-system organ failure; respiratory distress syndrome; acute myocardial infarction; myocardial infarction; status anginosus; status asthmaticus; status epilepticus; status lacunaris; inflammatory bowel disease; regional enteritis; ulcerative colitis; severe or debilitating arthritis; arthritis; psoriasis; severe psoriasis; burns; third degree burns; pancreatitis; acute pancreatitis; intestinal failure associated liver disease (IFALD); parenteral nutrition associated cholestasis (PNAC); essential fatty acid deficiency (EFAD); parenteral nutrition dependency complicated by soy allergy; local anesthetic systemic toxicity; treatment of systemic poisoning; need for a parenteral therapeutic drug vehicle or excipient; or allergy to lipid emulsions comprising ingredients other than MCTs and fish oil and/or omega-3 fatty acids. In some embodiments of any of the aspects, the patient administered a composition (e.g., an emulsion) described herein can be a subject having or diagnosed as having a condition selected from the group consisting of: hepatic steatosis; intestinal failure; parenteral nutrition-associated liver disease (PNALD); sepsis; cystic fibrosis; sickle cell anemia; pancreatitis; inflammatory bowel disease; Crohn's disease; biliary atresia; primary sclerosis cholangitis; an inflammatory infection; an inflammatory condition; systemic inflammatory response syndrome (SIRS); hypertriglyceridemia; severe hypertriglyceridemia; severe hepatic steatosis; retinopathy of prematurity; acute tubular necrosis; IgA nephropathies; ischemia-reperfusion injury; traumatic brain injury; multi-system organ failure; respiratory distress syndrome; acute myocardial infarction; myocardial infarction; status anginosus; status asthmaticus; status epilepticus; status lacunaris; inflammatory bowel disease; regional enteritis; ulcerative colitis; severe or debilitating arthritis; arthritis; psoriasis; severe psoriasis; burns; third degree burns; pancreatitis; acute pancreatitis; intestinal failure associated liver disease (IFALD); parenteral nutrition associated cholestasis (PNAC); essential fatty acid deficiency (EFAD); parenteral nutrition dependency complicated by soy allergy; local anesthetic systemic toxicity; treatment of systemic poisoning; or need for a parenteral therapeutic drug vehicle or excipient.

In some embodiments of any of the aspects, the subject administered a composition (e.g., an emulsion) as described herein has or is in need of treatment for a liver disease, e.g., fatty liver disease. As used herein “fatty-liver disease” refers to a disease wherein fat (hepatocytes) is excessively accumulated in the liver and can cause severe diseases such as chronic hepatitis and hepatic cirrhosis. In patients with fatty liver disease, lipids, particularly neutral fat, accumulate in hepatocytes to the extent that the amount exceeds the physiologically permissible range. From a biochemical point of view, a standard for judgment of fatty liver is that the weight of neutral fat is about 10% (100 mg/g wet weight) or more of the wet weight of hepatic tissue. Fatty liver disease is generally detected by observation of elevated serum levels of liver-specific enzymes such as the transaminases ALT and AST, which serve as indices of hepatocyte injury, as well as by presentation of symptoms, which include fatigue and pain in the region of the liver, though definitive diagnosis often requires a biopsy.

In some embodiments of any of the aspects, a subject administered a composition (e.g., an emulsion) as described herein has or is in need of treatment for PN associated or induced liver disease. This disease includes both biochemical, i.e., elevated serum aminotransferase, bilirubin, and alkaline phosphatase, and histologic alterations such as steatosis, steatohepatitis, lipidosis, cholestasis, fibrosis, and cirrhosis. The disease may be progressive and worsen with the course of PN administration and appears to be more prevalent in the pediatric population. Additional risk factors for this condition include prematurity, low birth weight, long-term use, the lack of concomitant oral intake, sepsis, and multiple operative procedures. Overall, the severity of PN-induced liver pathology is thought to be inversely related to the age of the patient.

In some embodiments of any of the aspects, the composition (e.g., the emulsion) described herein is administered parenterally or intravenously.

In some embodiments of any of the aspects, the composition (e.g., the emulsion) described herein is administered by parenteral administration (PN). In some embodiments of any of the aspects, the composition (e.g., the emulsion) described herein is administered by total parenteral administration (TPN). In some embodiments of any of the aspects, the subject administered a composition (e.g., an emulsion) described herein is in need of parenteral administration (PN). In some embodiments of any of the aspects, the subject administered a composition (e.g., an emulsion) described herein is in need of total parenteral administration (TPN). Methods of administering lipid emulsions to patients for PN applications or therapeutic benefit are known in the art. Typically the compositions (e.g., the emulsions) are administered by infusion over a suitable period of time. Appropriate dosages and administration; regimens can readily be determined by one skilled in the clinical arts.

In some embodiments of any of the aspects, the subject administered a composition (e.g., an emulsion) described herein for PN or TPN, during the period of treatment in which they are administered the composition (e.g., the emulsion) (e.g., the period of days or weeks in which they are administered the composition (e.g., the emulsion)), is not administered and/or permitted any oral nutrition. In some embodiments of any of the aspects, the subject administered a composition (e.g., an emulsion) described herein for PN or TPN, during the period of treatment in which they are administered the composition (e.g., the emulsion) (e.g., the period of days or weeks in which they are administered the composition (e.g., the emulsion)), is not administered and/or permitted any oral nutrition comprising fats and/or fatty acids.

In some embodiments of any of the aspects, the subject administered a composition (e.g., an emulsion) described herein for PN or TPN, during the period of treatment in which they are administered the composition (e.g., the emulsion) (e.g., the period of days or weeks in which they are administered the composition (e.g., the emulsion)), is not administered any other parenteral formulations. In some embodiments of any of the aspects, the subject administered a composition (e.g., an emulsion) described herein for PN or TPN, during the period of treatment in which they are administered the composition (e.g., the emulsion) (e.g., the period of days or weeks in which they are administered the composition (e.g., the emulsion)), is not administered any other parenteral formulations comprising fats and/or fatty acids.

In some embodiments of any of the aspects, the subject administered a composition (e.g., an emulsion) described herein for, during the period of treatment in which they are administered the composition (e.g., the emulsion) (e.g., the period of days or weeks in which they are administered the composition (e.g., the emulsion)), is not administered and/or permitted any other nutritional sources of fats and/or fatty acids.

In some embodiments of any of the aspects, the subject administered a composition (e.g., an emulsion) described herein, during the period of treatment in which they are administered the composition (e.g., the emulsion) (e.g., the period of days or weeks in which they are administered the composition (e.g., the emulsion)), is not administered and/or permitted any other nutritional sources of essential fatty acids. In some embodiments of any of the aspects, the subject administered a composition (e.g., an emulsion) described herein, during the period of treatment in which they are administered the composition (e.g., the emulsion) (e.g., the period of days or weeks in which they are administered the composition (e.g., the emulsion)), is not administered and/or permitted any other oral nutritional sources of essential fatty acids. In some embodiments of any of the aspects, the subject administered a composition (e.g., an emulsion) described herein, during the period of treatment in which they are administered the composition (e.g., the emulsion) (e.g., the period of days or weeks in which they are administered the composition (e.g., the emulsion)), is not administered and/or permitted any other parenteral nutritional sources of essential fatty acids.

In some embodiments of any of the aspects, the composition (e.g, the emulsion) described herein is administered orally. In some embodiments of any of the aspects, the subject administered a composition (e.g, an emulsion) described herein is in need of oral nutition. Methods of administering lipid emulsions to patients orally are known in the art. Oral administrion of an emulsified form of a composition described herein is particularly advantageous, as this can improve absorption in the digestive system.

In some embodiments of any of the aspects, the subject administered a composition (e.g., an emulsion) described herein, during the period of treatment in which they are administered the composition (e.g., the emulsion) (e.g., the period of days or weeks in which they are administered the composition (e.g., the emulsion)), is not administered and/or permitted any other nutritional sources which would be sufficient to maintain a nutritional balance. In some embodiments of any of the aspects, the subject administered a composition (e.g., an emulsion) described herein, during the period of treatment in which they are administered the composition (e.g., the emulsion) (e.g., the period of days or weeks in which they are administered the composition (e.g., the emulsion)), is not administered and/or permitted any other oral/enteral nutritional sources which would be sufficient to maintain a nutritional balance. In some embodiments of any of the aspects, the subject administered a composition (e.g., an emulsion) described herein, during the period of treatment in which they are administered the composition (e.g., the emulsion) (e.g., the period of days or weeks in which they are administered the composition (e.g., the emulsion)), is not administered and/or permitted any other parenteral nutritional sources which would be sufficient to maintain a nutritional balance. As used herein, “nutritional balance” refers to the maintenance of growth, development, and the lack nutritional deficiencies by provision of appropriate nutrition. Nutritional balance meets each individual's requirements without excessive provision of any particular nutrient that may produce an adverse outcome.

In some embodiments of any of the aspects, the patient does not receive or is not administered dextrose. In some embodiments of any of the aspects, the patient does not receive or is not administered a dextrose solution.

As described elsewhere herein, the compositions (e.g., emulsions) described herein are demonstrated to be suitable for monotherapy, e.g., they do not induce retinopathy, bronchopulmonary dysplasia, perinatal sepsis, essential fatty acid deficiency, inflammation, and/or other nutritional deficiencies when administered as a monotherapy. This is a characteristic not shared by all fat/fatty acid compositions which may be otherwise suitable for administration (e.g., parenteral administration). Accordingly, in some embodiments of any of the aspects, the composition (e.g., the emulsion) described herein is administered as a monotherapy for the condition the subject is in need of treatment for. In some embodiments of any of the aspects, the composition (e.g., the emulsion) described herein is administered as a monotherapy for nutritional needs, e.g., an anti-inflammatory without significant nutritional value could be administered concurrently, but the composition (e.g., the emulsion) would still be a monotherapy as regards nutritional needs. In some embodiments of any of the aspects, the composition (e.g., the emulsion) described herein is administered as a monotherapy as regards fatty acids, e.g., no other source of fatty acids is administered to or consumed by the subject.

In some embodiments of any of the aspects, the subject is a newborn or neonate. As used herein, the term “newborn” refers to an infant from the time of birth through the 28th day of life. In some embodiments of any of the aspects, the newborn is a human infant. In the embodiment that the newborn is a premature birth, the 28^{th} day is extended to include the number of days of premature birth.

In some embodiments of any of the aspects, the subject is an infant. As used herein, the term “infant” refers to a young from the time of birth to one year of age.

In some embodiments of any of the aspects, the subject is a premature newborn or infant. As used herein, the term “premature” refers to a human born after a gestation period of less than 38 weeks. In some embodiments of any of the aspects, a gestation period of less than 38 weeks refers to a menstrual age or gestational age of less than 38 weeks.

In some embodiments of any of the aspects, the subject is a premature newborn or infant born between 34 and 36 weeks of pregnancy (late preterm). In some embodiments of any of the aspects, the subject is a premature newborn or infant born between 32 and 34 weeks of pregnancy (moderately preterm). In some embodiments of any of the aspects, the subject is a premature newborn or infant born before 32 weeks of pregnancy (very preterm). In some embodiments of any of the aspects, the subject is a premature newborn or infant born at or before 25 weeks of pregnancy (extremely preterm).

In the case of premature infants, nutrition consideration and correction for gestational age is done up to the age of 2 years corrected age (not chronological age).

The last weeks of gestation provide critical inputs and nutrients required for neurological, retinal, and bronchopulmonary development. As demonstrated herein, administration of the compositions (e.g., emulsions) described herein are capable of effectively providing nutrition, modulating fatty acid levels and proportions in desirable ways, and reducing toxic effects. Additionally, administration of the compositions described herein can provide these beneficial and therapeutic effects across the blood-brain barrier, which is understood to reflect the effects experienced in the eye across the structurally similar blood-retinal barrier. Accordingly, described herein are therapeutic methods of treating premature birth, comprising administering a composition as described herein, e.g, according to a method described herein. In some aspects of any of the embodiments described herein are therapeutic methods of treating intraventricular hemorrhage, developmental cognitive disability, developmental delay, speech or language impairment, or vision impairment, comprising administering a composition as described herein, e.g, according to a method described herein. In some aspects of any of the embodiments described herein are therapeutic methods of treating intraventricular hemorrhage, developmental cognitive disability, developmental delay, speech or language impairment, or vision impairment, in a subject who was born prematurely, comprising administering a composition as described herein, e.g, according to a method described herein. In some aspects of any of the embodiments described herein are therapeutic methods of treating a neurological developmental deficit, comprising administering a composition as described herein, e.g, according to a method described herein. In some aspects of any of the embodiments described herein are therapeutic methods of treating a retinal developmental deficit, comprising administering a composition as described herein, e.g, according to a method described herein. In some aspects of any of the embodiments described herein are therapeutic methods of treating a bronchopulmonary developmental deficit, comprising administering a composition as described herein, e.g, according to a method described herein. In some aspects of any of the embodiments described herein are therapeutic methods of treating bronchopulmonary dysplasia, comprising administering a composition as described herein, e.g, according to a method described herein.

In some aspects of any of the embodiments, described herein is a composition (e.g., an emulsion) as described herein for use as a medicament. In some aspects of any of the embodiments, described herein is a composition (e.g., an emulsion) as described herein for use as a medicament in the treatment of premature birth. In some aspects of any of the embodiments, described herein is a composition (e.g., an emulsion) as described herein for use as a medicament in the treatment of a neurological developmental deficit. In some aspects of any of the embodiments, described herein is a composition (e.g., an emulsion) as described herein for use as a medicament in the treatment of a retinal developmental deficit. In some aspects of any of the embodiments, described herein is a composition (e.g., an emulsion) as described herein for use as a medicament in the treatment of a bronchopulmonary developmental deficit. In some aspects of any of the embodiments, described herein is a composition (e.g., an emulsion) as described herein for use as a medicament in the treatment of bronchopulmonary dysplasia. In some aspects of any of the embodiments, described herein is a composition (e.g., an emulsion) as described herein for use as a medicament in the treatment of premature birth. In some aspects of any of the embodiments, described herein is a composition (e.g., an emulsion) as described herein for use as a medicament in the treatment of intraventricular hemorrhage, developmental cognitive disability, developmental delay, speech or language impairment, or vision impairment. In some aspects of any of the embodiments, described herein is a composition (e.g., an emulsion) as described herein for use as a medicament in the treatment of intraventricular hemorrhage, developmental cognitive disability, developmental delay, speech or language impairment, or vision impairment, in a subject who was born prematurely.

In some aspects of any of the embodiments, described herein is a composition (e.g., an emulsion) as described herein for use as a non-therapeutic supplement. In some aspects of any of the embodiments, described herein is a composition (e.g., an emulsion) as described herein for use in a non-therapeutic method of improving or increasing the rate of brain development. In some aspects of any of the embodiments, described herein is a composition (e.g., an emulsion) as described herein for use in a non-therapeutic method of improving or increasing the rate of retinal development. In some aspects of any of the embodiments, described herein is a composition (e.g., an emulsion) as described herein for use in a non-therapeutic method of improving or increasing the rate of bronchopulmonary development. In some aspects of any of the embodiments, described herein is a composition (e.g., an emulsion) as described herein for use in a non-therapeutic method of improving or increasing the rate of infant growth and/or development.

Premature infants born at less than 34 weeks of pregnancy cannot adequately digest orally administered formulations. Accordingly, in one embodiment of any of the aspects, described herein is a method comprising parenterally administering a composition described herein to a premature infant born before 34 weeks of pregnancy. In one embodiment of any of the aspects, described herein is a method comprising parenterally administering a composition described herein to a premature infant born before 34 weeks of pregnancy, from birth until the age equivalent to 38 weeks of pregnancy. In one embodiment of any of the aspects, described herein is a method comprising parenterally administering a composition described herein to a premature infant born before 34 weeks of pregnancy and not administering the composition described herein orally. In one embodiment of any of the aspects, described herein is a method comprising parenterally administering a composition described herein to a premature infant born before 34 weeks of pregnancy, from birth until the age equivalent to 38 weeks of pregnancy, and not administering the composition described herein orally from birth until the age equivalent to 38 weeks of pregnancy.

In one embodiment of any of the aspects, described herein is a method comprising parenterally administering a composition described herein to a premature infant born before 34 weeks of pregnancy, from birth until the age equivalent to 38 weeks of pregnancy and then orally and/or parenterally administering the composition described herein (e.g,. from the age equivalent to 38 weeks of pregnancy until up to 2 years of age). In one embodiment of any of the aspects, described herein is a method comprising parenterally administering a composition described herein to a premature infant born before 34 weeks of pregnancy, from birth until the age equivalent to 38 weeks of pregnancy, and not administering the composition described herein orally from birth until the age equivalent to 37 weeks of pregnancy and then orally and/or parenterally administering the composition described herein (e.g,. from the age equivalent to 38 weeks of pregnancy until up to 2 years of age).

In one embodiment of any of the aspects, described herein is a method comprising parenterally administering a composition described herein to a premature infant born before 34 weeks of pregnancy, from birth until the age equivalent to 38 weeks of pregnancy and then orally administering the composition described herein (e.g,. from the age equivalent to 38 weeks of pregnancy until up to 2 years of age). In one embodiment of any of the aspects, described herein is a method comprising parenterally administering a composition described herein to a premature infant born before 34 weeks of pregnancy, from birth until the age equivalent to 38 weeks of pregnancy, and not administering the composition described herein orally from birth until the age equivalent to 38 weeks of pregnancy and then orally administering the composition described herein (e.g,. from the age equivalent to 38 weeks of pregnancy until up to 2 years of age).

Premature infants born between 34 and 38 weeks of pregnancy may be capable of digesting orally administered formulations, but may not be sufficiently efficient at digestion for only oral administration. Accordingly, in one embodiment of any of the aspects, described herein is a method comprising parenterally and/or orally administering a composition described herein to a premature infant born between 34 and 38 weeks of pregnancy. In one embodiment of any of the aspects, described herein is a method comprising parenterally and/or orally administering a composition described herein to a premature infant born between 34 and 38 weeks of pregnancy, from birth until the age equivalent to 38 weeks of pregnancy. In one embodiment of any of the aspects, described herein is a method comprising parenterally administering a composition described herein to a premature infant born between 34 and 38 weeks of pregnancy. In one embodiment of any of the aspects, described herein is a method comprising parenteraly administering a composition described herein to a premature infant born between 34 and 38 weeks of pregnancy, from birth until the age equivalent to 38 weeks of pregnancy. In one embodiment of any of the aspects, described herein is a method comprising orally administering a composition described herein to a premature infant born between 34 and 38 weeks of pregnancy. In one embodiment of any of the aspects, described herein is a method comprising orally administering a composition described herein to a premature infant born between 34 and 38 weeks of pregnancy, from birth until the age equivalent to 38 weeks of pregnancy. in one embodiment of any of the aspects, described herein is a method comprising parenterally and orally administering a composition described herein to a premature infant born between 34 and 38 weeks of pregnancy. In one embodiment of any of the aspects, described herein is a method comprising parenterally and orally administering a composition described herein to a premature infant born between 34 and 38 weeks of pregnancy, from birth until the age equivalent to 38 weeks of pregnancy.

For all infants, either those birthed at or after 38 weeks of pregnancy or those both premature, oral administration can be sufficient after the age equivalent to 38 weeks of pregnancy. In one embodiment of any of the aspects, described herein is a method comprising orally administering a composition described herein to an infant after the age equivalent to 38 weeks of pregnancy.

In some embodiments of any of the aspects, a composition (e.g., an emulsion) described herein can be administered at a dose of from about 0.5 g fatty acids/kg/day to about 5 g fatty acids/kg/day. In some embodiments of any of the aspects, a composition (e.g., an emulsion) described herein can be administered at a dose of from 0.5 g fatty acids/kg/day to 5 g fatty acids/kg/day. In some embodiments of any of the aspects, a composition (e.g., an emulsion) described herein can be administered at a dose of from about 1 g fatty acids/kg/day to about 3 g fatty acids/kg/day. In some embodiments of any of the aspects, a composition (e.g., an emulsion) described herein can be administered at a dose of from 1 g fatty acids/kg/day to 3 g fatty acids/kg/day. In some embodiments of any of the aspects, a composition (e.g., an emulsion) described herein can be administered at a dose of about 2 g fatty acids/kg/day.

In some embodiments of any of the aspects, a composition (e.g., an emulsion) described herein can be administered at a dose of no more than 5 g fatty acids/kg/day. In some embodiments of any of the aspects, a composition (e.g., an emulsion) described herein can be administered at a dose of no more than 4 g fatty acids/kg/day. In some embodiments of any of the aspects, a composition (e.g., an emulsion) described herein can be administered at a dose of no more than 3 g fatty acids/kg/day. In some embodiments of any of the aspects, a composition (e.g., an emulsion) described herein can be administered at a dose of no more than 2 g fatty acids/kg/day. In some embodiments of any of the aspects, a composition (e.g., an emulsion) described herein can be administered at a dose of no more than 1 g fatty acids/kg/day.

In some embodiments of any of the aspects, a composition (e.g., an emulsion) described herein is administered at a dose of 5-200 mg/kg/day of ARA and/or 10 mg/kg/day-3 g/kg/day of DHA and/or EPA. In some embodiments of any of the aspects, a composition (e.g., an emulsion) described herein is administered at a dose of 5-200 mg/kg/day of ARA. In some embodiments of any of the aspects, a composition (e.g., an emulsion) described herein is administered at a dose of 10 mg/kg/day-3 g/kg/day of DHA and/or EPA. In some embodiments of any of the aspects, a composition (e.g., an emulsion) described herein is administered at a dose of 5-200 mg/kg/day of ARA and 10 mg/kg/day-3 g/kg/day of DHA and/or EPA.

In some embodiments of any of the aspects, a composition (e.g., an emulsion) described herein is administered at a dose of 20-60 mg/kg/day of ARA and/or 40-100 mg/kg/day of DHA and/or EPA. In some embodiments of any of the aspects, a composition (e.g., an emulsion) described herein is administered at a dose of 20-60 mg/kg/day of ARA. In some embodiments of any of the aspects, a composition (e.g., an emulsion) described herein is administered at a dose of 40-100 mg/kg/day of DHA and/or EPA. In some embodiments of any of the aspects, a composition (e.g., an emulsion) described herein is administered at a dose of 20-60 mg/kg/day of ARA and 40-100 mg/kg/day of DHA and/or EPA.

In some embodiments of any of the aspects, a composition (e.g., an emulsion) described herein is administered at a dose of 10-120 mg/kg/day of ARA and/or 20-200 mg/kg/day of DHA and/or EPA. In some embodiments of any of the aspects, a composition (e.g., an emulsion) described herein is administered at a dose of 10-120 mg/kg/day of ARA. In some embodiments of any of the aspects, a composition (e.g., an emulsion) described herein is administered at a dose of 20-200 mg/kg/day of DHA and/or EPA. In some embodiments of any of the aspects, a composition (e.g., an emulsion) described herein is administered at a dose of 10-120 mg/kg/day of ARA and 20-200 mg/kg/day of DHA and/or EPA.

In some embodiments of any of the aspects, a composition (e.g., an emulsion) described herein is administered at a dose of 5-200 mg/kg/day of ARA and/or 10-300 mg/kg/day of DHA and/or EPA. In some embodiments of any of the aspects, a composition (e.g., an emulsion) described herein is administered at a dose of 5-200 mg/kg/day of ARA. In some embodiments of any of the aspects, a composition (e.g., an emulsion) described herein is administered at a dose of 10-300 mg/kg/day of DHA and/or EPA. In some embodiments of any of the aspects, a composition (e.g., an emulsion) described herein is administered at a dose of 5-200 mg/kg/day of ARA and 10-300 mg/kg/day of DHA and/or EPA. In some embodiments of any of the aspects, a composition (e.g., an emulsion) described herein is administered at a dose of 5-200 mg/kg/day of ARA and 10-400 mg/kg/day of DHA and/or EPA. In some embodiments of any of the aspects, a composition (e.g., an emulsion) described herein is administered at a dose of up to 200 mg/kg/day of ARA and up to 400 mg/kg/day of DHA and/or EPA.

In some embodiments of any of the aspects, a composition (e.g., an emulsion) described herein is administered at a dose of 5-200 mg/kg/day of ARA and/or 10-750 mg/kg/day of DHA and/or EPA. In some embodiments of any of the aspects, a composition (e.g., an emulsion) described herein is administered at a dose of 5-200 mg/kg/day of ARA. In some embodiments of any of the aspects, a composition (e.g., an emulsion) described herein is administered at a dose of 10-750 mg/kg/day of DHA and/or EPA. In some embodiments of any of the aspects, a composition (e.g., an emulsion) described herein is administered at a dose of 5-200 mg/kg/day of ARA and 10-750 mg/kg/day of DHA and/or EPA.

As described elsewhere herein, infants are sensitive to total administered volumes of formulations. For example, the current clinical standards for use of OMEGAVEN typically require a daily administered volume of approximately 10 cc/kg/day, which is higher than desired. In some embodiments of any of the aspects, the compositions and methods described herein relate to administering a total daily volume of a composition described herein of less than 10 cc/kg/day. In some embodiments of any of the aspects, the compositions and methods described herein relate to administering a total daily volume of a composition described herein of less than 9 cc/kg/day. In some embodiments of any of the aspects, the compositions and methods described herein relate to administering a total daily volume of a composition described herein of less than 8 cc/kg/day. In some embodiments of any of the aspects, the compositions and methods described herein relate to administering a total daily volume of a composition described herein of less than 7 cc/kg/day. In some embodiments of any of the aspects, the compositions and methods described herein relate to administering a total daily volume of a composition described herein of less than 6 cc/kg/day. In some embodiments of any of the aspects, the compositions and methods described herein relate to administering a total daily volume of a composition described herein of less than 5 cc/kg/day. In some embodiments of any of the aspects, the compositions and methods described herein relate to administering a total daily volume of a composition described herein of less than 4 cc/kg/day. In some embodiments of any of the aspects, the compositions and methods described herein relate to administering a total daily volume of a composition described herein of less than 3 cc/kg/day. In some embodiments of any of the aspects, the compositions and methods described herein relate to administering a total daily volume of a composition described herein of less than 2 cc/kg/day.

In some embodiments of any of the aspects, a composition (e.g., an emulsion) described herein can be administered at a dose of no more than 3 g fatty acids/kg/day and a total volume of no more than 5 cc/kg/day.

In some embodiments of any of the aspects, the administration of a composition (e.g., an emulsion) as described herein is continued for at least 3 days, e.g., 3 or more days, 4 or more days, 5 or more days, 7 or more days, 2 or more weeks, 3 or more weeks, 4 or more weeks, 6 or more weeks, 2 months or more, or 3 months or more. In some embodiments of any of the aspects, the administration of a composition (e.g., an emulsion) as described herein is continued for at least 3 weeks. In some embodiments of any of the aspects, the administration of a composition (e.g., an emulsion) as described herein is continued for at least 6 weeks. In some embodiments of any of the aspects, the administration of a composition (e.g., an emulsion) as described herein is continued for at least 3 months.

The compositions and methods described herein can be administered to a subject having or diagnosed as having a condition described herein. In some embodiments, the methods described herein comprise administering an effective amount of compositions described herein, e.g. a composition (e.g., an emulsion) to a subject in order to alleviate a symptom of a condition described herein. As used herein, “alleviating a symptom” is ameliorating any condition or symptom associated with the condition. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to oral, parenteral, or intravenous administration.

In some embodiments of any of the aspects, described herein is a prophylactic method of treatment. As used herein “prophylactic” refers to the timing and intent of a treatment relative to a disease or symptom, that is, the treatment is administered prior to clinical detection or diagnosis of that particular disease or symptom in order to protect the patient from the disease or symptom. Prophylactic treatment can encompass a reduction in the severity or speed of onset of the disease or symptom, or contribute to faster recovery from the disease or symptom. Accordingly, the methods described herein can be prophylactic relative to retinopathy, bronchopulmonary dysplasia, or perinatal sepsis. In some embodiments of any of the aspects, prophylactic treatment is not prevention of all symptoms or signs of a disease.

The term “effective amount” as used herein refers to the amount of a composition (e.g., an emulsion) described herein needed to alleviate at least one or more symptom of the disease or disorder, and relates to a sufficient amount of pharmacological composition to provide the desired effect. The term “therapeutically effective amount” therefore refers to an amount of a composition (e.g., an emulsion) described herein that is sufficient to provide a particular effect (e.g., nutritional, or anti-retinopathy, anti-bronchopulmonary dysplasia, or anti-perinatal sepsis effect) when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the LD50 (the dose lethal to 50% of the population) and the ED50 (the dose therapeutically effective in 50% of the population). The dosage can vary depending upon the dosage form employed and the route of administration utilized. The dose ratio between toxic and therapeutic effects is the therapeutic index and can be expressed as the ratio LD50/ED50. Compositions and methods that exhibit large therapeutic indices are preferred. A therapeutically effective dose can be estimated initially from cell culture assays. Also, a dose can be formulated in animal models to achieve a circulating plasma concentration range that includes the IC50 (i.e., the concentration of the composition (e.g., the emulsion) or components thereof, which achieves a half-maximal inhibition of symptoms) as determined in cell culture, or in an appropriate animal model. Levels in plasma can be measured, for example, by high performance liquid chromatography. The effects of any particular dosage can be monitored by a suitable bioassay, e.g., assay for liver function among others. The dosage can be determined by a physician and adjusted, as necessary, to suit observed effects of the treatment.

In some embodiments, the technology described herein relates to a pharmaceutical composition comprising a composition (e.g., an emulsion) described herein as described herein, and optionally a pharmaceutically acceptable carrier. In some embodiments, the active ingredients of the pharmaceutical composition comprise a composition (e.g., an emulsion) described herein as described herein. In some embodiments, the active ingredients of the pharmaceutical composition consist essentially of a composition (e.g., an emulsion) described herein as described herein. In some embodiments, the active ingredients of the pharmaceutical composition consist of a composition (e.g., an emulsion) described herein as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose, dextrose, and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) glycols, such as propylene glycol; (10) polyols, such as glycerin, sorbitol, mannitol and polyethylene glycol (PEG); (11) esters, such as ethyl oleate and ethyl laurate; (12) agar; (13) buffering agents, such as magnesium hydroxide and aluminum hydroxide; (14) alginic acid; (15) pyrogen-free water; (16) isotonic saline; (17) Ringer's solution; (18) ethyl alcohol; (19) pH buffered solutions; (20) polyesters, polycarbonates and/or polyanhydrides; (21) bulking agents, such as polypeptides and amino acids (22) serum component, such as serum albumin, HDL and LDL; (23) C₂-C₁₂ alcohols, such as ethanol; and (24) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the degradation of the active agents as described herein. It is noted that the composition (e.g., the emulsion) itself can comprise water. The composition (e.g., the emulsion) can be administered with other components used in parenteral nutrition solutions (e.g. dextrose, crystalline amino acids, trace elements, multivitamins, electrolytes and minerals).

In some embodiments, the pharmaceutical composition comprising a composition (e.g., an emulsion) described herein as described herein can be a parenteral dose form. Since administration of parenteral dosage forms typically bypasses the patient's natural defenses against contaminants, parenteral dosage forms are preferably sterile or capable of being sterilized prior to administration to a patient. Examples of parenteral dosage forms include, but are not limited to, solutions ready for injection, dry products ready to be dissolved or suspended in a pharmaceutically acceptable vehicle for injection, suspensions ready for injection, and emulsions. In addition, controlled-release parenteral dosage forms can be prepared for administration of a patient, including, but not limited to, DUROS®-type dosage forms and dose-dumping.

Pharmaceutical compositions comprising a composition (e.g., an emulsion) described herein can also be formulated to be suitable for oral administration, for example as discrete dosage forms, such as, but not limited to, tablets (including without limitation scored or coated tablets), pills, caplets, capsules, chewable tablets, powder packets, cachets, troches, wafers, or liquids, such as but not limited to, syrups, elixirs, solutions or suspensions in an aqueous liquid, a non-aqueous liquid, an oil-in-water emulsion, or a water-in-oil emulsion. Such compositions contain a predetermined amount of the pharmaceutically acceptable salt of the disclosed compounds, and may be prepared by methods of pharmacy well known to those skilled in the art. See generally, Remington: The Science and Practice of Pharmacy, 21st Ed., Lippincott, Williams, and Wilkins, Philadelphia PA. (2005).

The methods described herein can further comprise administering a second agent and/or treatment to the subject, e.g. as part of a combinatorial therapy. By way of non-limiting example the subject can also be administered a second agent and/or treatment known to be beneficial for subjects suffering from inflammation. Examples of such agents and/or treatments include, but are not limited to, non-steroidal anti-inflammatory drugs (NSAIDs-such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising a composition (e.g., an emulsion) described herein as described herein can be administered to a patient once. In certain embodiments, an effective dose of a composition comprising a composition (e.g., an emulsion) described herein can be administered to a patient repeatedly, e.g., daily or several times a day for a period of at least 3 days, at least 4 days, at least 5 days, at least 6 days, at least 1 week, at least 2 weeks, at least 3 week, at least 4 weeks, at least 6 weeks, at least 2 months, or at least 3 months.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the components of the composition (e.g., the emulsion). The desired dose or amount of activation can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising a composition (e.g., an emulsion) described herein can be administered over a period of time, such as over a 5 minute, 10 minute, 15 minute, 20 minute, or 25 minute period.

The dosage ranges for the administration of a composition (e.g., an emulsion) described herein, according to the methods described herein depend upon, for example, the form of the composition (e.g., the emulsion), its potency, and the extent to which symptoms, markers, or indicators of a condition described herein are desired to be reduced. The dosage should not be so large as to cause adverse side effects. Generally, the dosage will vary with the age, condition, and sex of the patient and can be determined by one of skill in the art. The dosage can also be adjusted by the individual physician in the event of any complication.

The efficacy of a composition (e.g., an emulsion) described herein in, e.g. the treatment of a condition described herein, or to induce a response as described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g. nutritional balance, inflammation, and/or liver function. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g. pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein. When using an experimental animal model, efficacy of treatment is evidenced when a statistically significant change in a marker is observed.

In vitro and animal model assays are provided herein which allow the assessment of a given dose of a composition (e.g., an emulsion) as described herein. By way of non-limiting example, the effects of a dose of a composition (e.g., an emulsion) can be assessed by administering the composition (e.g., the emulsion) orally or parenterally and then assessing serum fatty acid levels, and liver, spleen, and/or kidney histology.

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

For convenience, certain terms employed herein, in the specification, examples and appended claims are collected here.

In one respect, the present invention relates to the herein described compositions, methods, and respective component(s) thereof, as essential to the technology, yet open to the inclusion of unspecified elements, essential or not (“comprising). In some embodiments of any of the aspects, other elements to be included in the description of the composition, method or respective component thereof are limited to those that do not materially affect the basic and novel characteristic(s) of the technology (e.g., the composition, method, or respective component thereof “consists essentially of” the elements described herein). This applies equally to steps within a described method as well as compositions and components therein. In other embodiments of any of the aspects, the compositions, methods, and respective components thereof, described herein are intended to be exclusive of any element not deemed an essential element to the component, composition or method (e.g., the composition, method, or respective component thereof “consists of” the elements described herein). This applies equally to steps within a described method as well as compositions and components therein.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, a “increase” is a statistically significant increase in such level.

As used herein, a “subject” means a human or animal. Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of the diseases and conditions described herein. A subject can be male or female.

A subject can be one who has been previously diagnosed with or identified as suffering from or having a condition in need of treatment or one or more complications related to such a condition, and optionally, have already undergone treatment for the condition or the one or more complications related to the condition. Alternatively, a subject can also be one who has not been previously diagnosed as having the condition or one or more complications related to the condition. For example, a subject can be one who exhibits one or more risk factors for the condition or one or more complications related to the condition or a subject who does not exhibit risk factors.

A “subject in need” of treatment for a particular condition can be a subject having that condition, diagnosed as having that condition, or at risk of developing that condition.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. a condition, disease or disorder as described herein. The term “treating” includes reducing or alleviating at least one adverse effect or symptom of a condition, disease or disorder. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be an emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in in nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in immunology and molecular biology can be found in The Merck Manual of Diagnosis and Therapy, 19th Edition, published by Merck Sharp & Dohme Corp., 2011 (ISBN 978-0-911910-19-3); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), Taylor & Francis Limited, 2014 (ISBN 0815345305, 9780815345305); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

In some embodiments, the present technology may be defined in any of the following numbered paragraphs:

1. A composition comprising:
a) Arachidonic acid (ARA); and
b) Docosahexaenoic acid (DHA) and/or Eicosapentaenoic acid (EPA).

2. The composition of paragraph 1, comprising DHA.

3. The composition of paragraph 1, comprising EPA.

4. The composition of paragraph 1, comprising DHA and EPA.

5. The composition of any one of the preceding paragraphs, wherein the ratio of a) ARA to b) DHA and/or EPA is from 3:2 to 3:5 by weight.

6. The composition of any one of the preceding paragraphs, wherein the ratio of a) ARA to b) DHA and/or EPA is from 1:2 to 3:5 by weight.

7. The composition of any one of the preceding paragraphs, wherein the ratio of a) ARA to b) DHA and/or EPA is from 1:2 to 1:5 by weight.

8. The composition of any one of the preceding paragraphs, wherein the ratio of a) ARA to b) DHA and/or EPA is from 2:1 to 1:4 by weight.

9. The composition of any one of the preceding paragraphs, wherein the ratio of a) ARA to b) DHA and/or EPA is from 1:1 to 1:2 by weight.

10. The composition of any one of the preceding paragraphs, wherein the composition comprises further omega-6 fatty acids and/or omega-3 fatty acids.

11. The composition of paragraph 10, wherein the ARA and DHA and/or EPA comprise at least 60% of the total omega-3 fatty acids and omega-6 fatty acids.

12. The composition of paragraph 10, wherein the ARA and DHA and/or EPA comprise at least 70% of the total omega-3 fatty acids and omega-6 fatty acids.

13. The composition of paragraph 10, wherein the ARA and DHA and/or EPA comprise at least 80% of the total omega-3 fatty acids and omega-6 fatty acids.

14. The composition of paragraph 10, wherein the ARA and DHA and/or EPA comprise at least 90% of the total omega-3 fatty acids and omega-6 fatty acids.

15. The composition of any one of the preceding paragraphs, wherein the ARA comprises at least 20% of the total omega-6 fatty acids.

16. The composition of any one of the preceding paragraphs, wherein the ARA comprises at least 30% of the total omega-6 fatty acids.

17. The composition of any one of the preceding paragraphs, wherein the ARA comprises at least 40% of the total omega-6 fatty acids.

18. The composition of any one of the preceding paragraphs, wherein the DHA and/or EPA comprises at least 20% of the total omega-3 fatty acids.

19. The composition of any one of the preceding paragraphs, wherein the DHA and/or EPA comprises at least 30% of the total omega-3 fatty acids.

20. The composition of any one of the preceding paragraphs, wherein the DHA and/or EPA comprises at least 40% of the total omega-3 fatty acids.

21. The composition of any one of the preceding paragraphs, wherein the ratio of omega-6 fatty acids to omega-3 fatty acids is from 2:1 to 1:4 by weight.

22. The composition of any one of the preceding paragraphs, wherein the ratio of omega-6 fatty acids to omega-3 fatty acids is from 1:1 to 1:2 by weight.

23. The composition of any one of the preceding paragraphs, wherein the composition and the components thereof are not distilled or re-esterified.

24. The composition of one of the preceding paragraphs, wherein the total triglyceride+diglyceride content of the composition comprises no more than 10% diglyceride.

25. The composition of any one of paragraphs 23-24, wherein the composition comprises no diglyceride.

26. The composition of any one of the preceding paragraphs, wherein the DHA and/or EPA is not distilled or re-esterified.

27. The composition of any one of the preceding paragraphs, wherein the total triglyceride+diglyceride content of the DHA and/or EPA comprises no more than 10% diglyceride.

28. The composition of any one of the preceding paragraphs, wherein the DHA and/or EPA comprises no diglyceride.

29. The composition of any one of the preceding paragraphs, wherein the ARA is not distilled or re-esterified.

30. The composition of any one of the preceding paragraphs, wherein the total triglyceride+diglyceride content of the ARA comprises no more than 10% diglyceride.

31. The composition of any one of the preceding paragraphs, wherein the ARA comprises no diglyceride.

32. The composition of any one of the preceding paragraphs, wherein the composition comprises no more than 5% (w/w) sterols.

33. The composition of any one of the preceding paragraphs, wherein the composition comprises no more than 2% (w/w) sterols.

34. The composition of any one of the preceding paragraphs, wherein the composition comprises no more than 1.5% (w/w) sterols.

35. The composition of any one of the preceding paragraphs, wherein the composition comprises no more than 70 mg/L phytosterols.

36. The composition of any one of the preceding paragraphs, wherein the composition comprises no more than 35 mg/L phytosterols.

37. The composition of any one of the preceding paragraphs, wherein the composition comprises no more than 4% (w/w) stigmasterol.

38. The composition of any one of the preceding paragraphs, wherein the composition comprises no more than 10 mg/L stigmasterol.

39. The composition of any one of the preceding paragraphs, wherein the composition comprises, or the ARA is provided in the form of a plant or fungal oil.

40. The composition of paragraph 39, wherein the plant or fungal oil is not distilled or re-esterified.

41. The composition of any one of paragraphs 39-40, wherein the total triglyceride+diglyceride content of the plant or fungal oil comprises no more than 10% diglyceride.

42. The composition of any one of paragraphs 39-40, wherein the plant or fungal oil comprises no diglyceride.

43. The composition of any one of paragraphs 39-42, wherein the plant or fungal oil comprises no more than 5% (w/w) sterols, no more than 70 mg/L phytosterols, no more than 4% (w/w) stigmasterol, and/or no more than 10 mg/L stigmasterol.

44. The composition of any one of paragraphs 39-43, wherein the plant oil is soybean oil or olive oil, or the fungal oil is Mortierella alpina oil.

45. The composition of any one of the preceding paragraphs, wherein the composition comprises a fish oil, and/or the DHA and/or EPA is provided in the form of fish oil.

46. The composition of paragraph 45, wherein the fish oil is not distilled or re-esterified.

47. The composition of any one of paragraphs 45-46, wherein the total triglyceride+diglyceride content of the fish oil comprises no more than 10% diglyceride.

48. The composition of any one of paragraphs 45-46, wherein the fish oil comprises no diglyceride.

49. The composition of any one of paragraphs 45-48, wherein the fish oil comprises no more than 5% (w/w) sterols, no more than 70 mg/L phytosterols, no more than 4% (w/w) stigmasterol, and/or no more than 10 mg/L stigmasterol.

50. The composition of any one of the preceding paragraphs, wherein the composition comprises an algal oil, and/or the DHA and/or EPA is provided in the form of algal oil.

51. The composition of paragraph 50, wherein the algal oil is oil from Crypthecodinium cohnii.

52. The composition of any one of paragraphs 50-51, wherein the algal oil is not distilled or re-esterified.

53. The composition of any one of paragraphs 50-52, wherein the total triglyceride+diglyceride content of the algal oil comprises no more than 10% diglyceride.

54. The composition of any one of paragraphs 50-52, wherein the algal oil comprises no diglyceride.

55. The composition of any one of paragraphs 50-54, wherein the algal oil comprises no more than 5% (w/w) sterols, no more than 70 mg/L phytosterols, no more than 4% (w/w) stigmasterol, and/or no more than 10 mg/L stigmasterol.

56. The composition of any one of the preceding paragraphs, wherein the composition is a lipid emulsion.

57. The composition of paragraph 56, wherein the emulsion is at least 10% oil in water.

58. The composition of paragraph 56, wherein the emulsion is at least 20% oil in water.

59. The composition of paragraph 56, wherein the emulsion is at least 30% oil in water.

60. The composition of paragraph 56, wherein the emulsion is about 10% to about 50% oil in water.

61. The composition of paragraph 56, wherein the emulsion is about 10% to about 40% oil in water.

62. The composition of paragraph 56, wherein the emulsion is about 10% to about 30% oil in water.

63. The composition of paragraph 56, wherein the emulsion is about 20% to about 40% oil in water.

64. The composition of any one of the preceding paragraphs, wherein the composition further comprises one or more of:
Medium chain triglycerides (MCTs); egg lecithin; sunflower seed oil; and an emulsifier obtained from sunflower seed.

65. The composition of any one of the preceding paragraphs, wherein the composition is formulated to comprise a) ARA at a dosage of 20-60 mg/kg/day and/or b) DHA and/or EPA at a dosage of 40-100 mg/kg/day.

66. The composition of any one of the preceding paragraphs, wherein the composition is formulated to comprise a) ARA at a dosage of 20-60 mg/kg/day and/or b) DHA and/or EPA at a dosage of 10 mg/kg/day-3 g/kg/day.

67. The composition of any one of the preceding paragraphs, wherein the composition is formulated for parenteral or intravenous administration.

68. A method of providing nutrition to a subject and/or promoting neurodevelopment in a subject, the method comprising administering the composition of any of the preceding paragraphs to the subject.

69. The method of any one of the preceding paragraphs, wherein the composition is administered at a dose of no more than 5 g/kg/day.

70. The method of any one of the preceding paragraphs, wherein the composition is administered to provide a) ARA at a dosage of 20-60 mg/kg/day and/or b) DHA and/or EPA at a dosage of 40-100 mg/kg/day.

71. The method of any one of the preceding paragraphs, wherein the subject is an infant.

72. The method of any one of the preceding paragraphs, wherein the subject is a neonatal and/or premature infant.

73. The method of any one of the preceding paragraphs, wherein the neurodevelopment is neurodevelopment in the brain and/or eyes.

74. The method of any one of the preceding paragraphs, whereby the administration treats, prevents, or reduces the risk of one or more conditions selected from the group consisting of: retinopathy; bronchopulmonary dysplasia; and perinatal sepsis.

75. The method of any one of the preceding paragraphs, wherein the administration is parenteral and/or intravenous.

76. The method of any one of the preceding paragraphs, wherein the nutrition is parenteral nutrition or total parenteral nutrition.

77. The method of any one of the preceding paragraphs, wherein the administration is parenteral administration or total parenteral administration.

78. The method of any one of the preceding paragraphs, wherein the subject is in need of parenteral nutrition or total parenteral nutrition.

79. The method of any of the preceding paragraphs, wherein the patient does not receive oral nutrition.

80. The method of any of the preceding paragraphs, wherein the patient does not receive other parenteral formulations.

81. The method of any of the preceding paragraphs, wherein the patient does not receive oral nutrition which is sufficient to maintain a nutritional balance.

82. The method of any of any of the preceding paragraphs, wherein the patient does not receive other parenteral formulations which are sufficient to maintain a nutritional balance.

83. The method of any of the preceding paragraphs, wherein the patient does not receive other nutritional sources and/or parenteral nutritional sources of fatty acids.

84. The method of any of the preceding paragraphs, wherein the patient does not receive other nutritional sources and/or parenteral nutritional sources of essential fatty acids.

85. The method of any of the preceding paragraphs, wherein the composition of any of paragraphs 1-67 is administered as a monotherapy.

86. The method of any of the preceding paragraphs, wherein the composition of any of paragraphs 1-67 is administered as a monotherapy for nutritional needs.

87. The method of any of the preceding paragraphs, wherein the patient is a patient in need of treatment for a condition selected from the group consisting of:
hepatic steatosis; intestinal failure; parenteral nutrition-associated liver disease (PNALD); sepsis; cystic fibrosis; sickle cell anemia; pancreatitis; inflammatory bowel disease; Crohn's disease; biliary atresia; primary sclerosis cholangitis; an inflammatory infection; an inflammatory condition; systemic inflammatory response syndrome (SIRS); hypertriglyceridemia; severe hypertriglyceridemia; severe hepatic steatosis; retinopathy of prematurity; acute tubular necrosis; IgA nephropathies; ischemia-reperfusion injury; traumatic brain injury; multi-system organ failure; respiratory distress syndrome; acute myocardial infarction; myocardial infarction; status anginosus; status asthmaticus; status epilepticus; status lacunaris; inflammatory bowel disease; regional enteritis; ulcerative colitis; severe or debilitating arthritis; arthritis; psoriasis; severe psoriasis; burns; third degree burns; pancreatitis; acute pancreatitis; intestinal failure associated liver disease (IFALD), parenteral nutrition associated cholestasis (PNAC), essential fatty acid deficiency (EFAD), parenteral nutrition dependency complicated by soy allergy.

The technology described herein is further illustrated by the following examples which in no way should be construed as being further limiting.

### EXAMPLES

### Example 1

Parenteral nutrition (PN) is a life-saving treatment for patients with intestinal failure from insufficient gastrointestinal length or function. While lifesaving, PN presents significant clinical challenges in the neonatal population.

First, the standard of care lipid emulsions (fat source in PN) used are low in docosahexanoic acid (DHA) and arachidonic acid (ARA), fatty acids that are critical for neurodevelopment and accumulate rapidly in the brain during the third trimester and first year of life.

Second, these emulsions contribute to a spectrum of cholestasis and liver failure known as intestinal failure-associated liver disease (IFALD).

Third, administering PN requires very high fluid volumes the typical PN volume for premature neonates is 130 mL/kg/day. Fluid administration in the neonate is already extremely challenging due to fluid shifts, physiological changes in kidney function, and high insensible losses

Fourth, in addition to IFALD, key complications of prematurity (retinopathy of prematurity and bronchopulmonary dysplasia) have also been linked to use of standard lipid emulsions No lipid emulsion has ever previously been designed for or specifically studied in the neonatal population, despite most neonatal ICU patients requiring PN.

Described herein is the preparation of a new parenteral lipid emulsion designed to (1) supply necessary docosahexaenoic acid and arachidonic acid for neonatal growth and development, (2) minimize phytosterols and prevent hepatic toxicity, (3) decrease inflammatory and neurological complications of prematurity including bronchopulmonary dysplasia, retinopathy of prematurity, and cerebral palsy.

The lipid emulsion will be a unique composition of several oils, including a source of arachidonic acid (e g. ARASCO oil), DHA and/or EPA (e g e.g fish oil or DHASCO oil), and low phytosterol oils (e.g., phytosterol reduced soybean oil, olive oil, or other low phytosterol oils). The ARA dose will be sufficient to provide 20-60 mg/kg/day based upon total lipid emulsion dosing, while the the DHA and/or EPA dose will range from 40-100 mg/kg/day based upon estimated fetal accretion rates and requirement for normal development (Robinson and Martin, Seminars in Fetal & Neonatal Medicine, 2017). In addition, the emulsion will be at least a 20% oil in water emulsion, and can be more concentrated (e.g 25% or 30%) to minimize fluid administration requirement.

### Example 2

### Experiment 1: Generation of New Lipid Emulsions for Premature Neonates

Purpose: Generate new lipid emulsions that pass USP<729> standards with a fatty acid composition that is anti-inflammatory (i.e. low omega 6:omega 3 ratio), hepatoprotective (low in phytosterols and high in alpha-tocopherol), and contain sufficient arachidonic acid and docosahexaenoic acid to support growth and development.

Methods: Three oils were obtained: fish oil (Lipoid GmbH, Ludwigshafen, Germany), phytosterol-depleted soybean oil (Lipoid GmbH, Ludwigshafen, Germany), and arachidonic acid-rich fungal oil derived from Mortierella alpine (Cargill, Inc., Minneapolis, MN). Each oil was individually prepared as a 20% oil-in-water lipid emulsions using high pressure homogenization. First, a dispersion was generated. Frozen egg phospholipid was dispersed in sterile water for injection under high shear mixing, followed by sodium oleate and glycerin. This dispersion was then passed through a high-pressure homogenizer (PandaPlus 2000, GEA, Parma, Italy) for 20 cycles at 5000 PSI and then 10 cycles at 9000 PSI. Next, the dispersion was filtered through a 0.45 μm membrane and pH adjusted to 10.2 to 10.8. Oil was then added to the dispersion under high shear mixing in a thin stream, followed by high pressure homogenization at 9000 PSI for 10 cycles and 13000 PSI for 5 cycles. The fish oil and fungal oil emulsions were supplemented with alpha-tocopherol during high shear mixing to 150 mg/L, similar to the content in the low phytosterol soybean oil. The emulsion was then pH adjusted to 8.8-9.2 and bottled into sterile vials, which were subsequently autoclaved. All steps occurred under a nitrogen atmosphere to prevent peroxidation of the lipids. The final composition of the lipid emulsions was 200 g/L oil, 12 g/L egg phospholipid, 0.3 g/L sodium oleate, 25 g/L glycerin, and sterile water for injection. The lipid emulsions were sent to an independent laboratory to evaluate for USP <729> compliance (Micro Measurement Labs, Wheeling, IL). Following this, three new lipid emulsions (henceforth NLE A, NLE B, NLE C) were generated by mixing different ratios of the three single oil lipid emulsions, which were rebottled under sterile conditions with a nitrogen headspace and then autoclaved.

Results: Three 20% oil-in-water lipid emulsions were generated: fish oil lipid emulsion, low phytosterol soybean oil lipid emulsion, and arachidonic acid-rich lipid emulsion. All three lipid emulsions passed method I and II defined under USP <729> (Table 1). Following this, three new lipid emulsions were generated with varying ratios of the three single oil lipid emulsions. Table 2 shows the fatty acid composition of each new lipid emulsion. The three lipid emulsions (NLE A, B, and C) provided at 3 g/kg/day meet or exceed recommended minimum arachidonic acid intake (35-45 mg/kg/day) at 34.3 mg, 102.83 mg, and 137.1 mg respectively (dx.doi.org/10.1016/j.siny.2016.08.009). Similarly, NLE A, B, and C well exceed recommend minimum docosahexaenoic acid intake (55-60 mg/kg/day) at 230.6 mg, 195.1 mg, and 144.9 mg respectively.

TABLE 1
USP <729> Injectable lipid emulsion testing for fish oil, low phytosterol
soybean oil, and ARA-rich fungal oil lipid emulsions. All lipid emulsions passed
standards for PFAT % (pass < 0.05%) and DLS mean diameter (<500 nm).

| | | # of particles ≥ 5.02 um | | | | | | DLS/NICOMP Mean | | |
|---|---|---|---|---|---|---|---|---|---|---|
| | | but ≤ 50.45 um | | | PFAT5% | | | Diameter (nm) | | |
| Emulsion | Lot # | Run 1 | Run 2 | Run 3 | Run 1 | Run 2 | Run 3 | Run 1 | Run 2 | Run 3 |
| 20% Fish Oil Emulsion | 22-04 | 7,466 | 15,984 | 25,152 | 0.0358 | 0.0373 | 0.0386 | 237.3 | 240.4 | 239.6 |
| 20% Low Phytosterol | 22-05 | 1,678 | 4,089 | 5,945 | 0.0328 | 0.0350 | 0.0353 | 270.5 | 265.2 | 274.9 |
| Soybean Oil Emulsion | | | | | | | | | | |
| 20% ARA-rich Fungal | 22-06 | 1,686 | 3,392 | 4,979 | 0.0207 | 0.0114 | 0.0117 | 257.6 | 255.8 | 286.2 |
| Oil Emulsion | | | | | | | | | | |

TABLE 2
New lipid emulsions were generating using different propotions
of fish oil, low phytosterol soybean oil, and ARA fungal oil.

| Oil | NLE A | NLE B | NLE C |
|---|---|---|---|
| Fish Oil | 80% | 67.50% | 50% |
| Low Phytosterol Soy | 17.50% | 25% | 40% |
| ARA Fungal Oil | 2.50% | 7.50% | 10% |

TABLE 3
Fatty acid composition and tocopherol content of the three constituent oils and the new
lipid emulsions. Fatty acid composition and tocopherol testing of oils used in new lipid
emulsions was performed by Eurofins Microbiology Laboratories, North Kingstown, RI.

| | | | Low | | | | |
|---|---|---|---|---|---|---|---|
| | | Lipoid | Phytosterol | ARA | | | |
| | | Fish | Soybean | Fungal | | | |
| | | Oil | Oil | Oil | NLE A | NLE B | NLE C |
| Fatty Acid Composition % | | | | | | | |
| c14:0 | Myristic | 6.62% | 0.06% | 0.30% | 5.31% | 4.51% | 3.36% |
| c16:0 | Palmitic | 13.10% | 8.99% | 6.74% | 12.22% | 11.60% | 10.82% |
| c18:0 | Stearic | 2.37% | 3.83% | 5.65% | 2.71% | 2.98% | 3.28% |
| c18:1 | Vaccenic | 2.68% | 1.35% | 0.29% | 2.39% | 2.17% | 1.91% |
| c18:1 | Oleic | 7.39% | 20.10% | 8.76% | 9.65% | 10.67% | 12.61% |
| c18:2 | Linoleic | 1.04% | 49.40% | 4.45% | 9.59% | 13.39% | 20.73% |
| c18:3 | aLA | 0.53% | 5.79% | 0.03% | 1.44% | 1.81% | 2.58% |
| c20:4 | ARA | 0.80% | <0.02% | 41.74% | 1.68% | 3.67% | 4.57% |
| c20:5 | EPA | 15.73% | <0.02% | <0.06% | 12.58% | 10.62% | 7.87% |
| c22:6 | DHA | 10.58% | 0.03% | <0.02% | 8.47% | 7.15% | 5.30% |
| | total n3 | 32.11% | 5.89% | 0.22% | 26.72% | 23.16% | 18.43% |
| | total n6 | 2.85% | 49.52% | 53.79% | 12.29% | 18.34% | 26.61% |
| | total n9 | 8.44% | 20.54% | 9.76% | 10.59% | 11.56% | 13.41% |
| | n6:n3 | 0.09 | 8.41 | 244.50 | 0.46 | 0.79 | 1.44 |
| | ratio | | | | | | |
| Tocopherol Content mg/100 g | | | | | | | |
| Alpha | | 6.09 | 68.50 | 95.60 | 75.25 | 75.66 | 75.01 |
| tocopherol | | | | | | | |
| Total | | 6.09 | 70.60 | 95.60 | 75.62 | 76.18 | 75.85 |
| tocopherols | | | | | | | |

TABLE 4
Fatty acid provision of new lipid emulsions at 3 g/kg/d dosing.

| | | NLE A | NLE B | NLE C |
|---|---|---|---|---|
| Fatty Acids | | mg/kg/d at 3 g/kg dosing | | |
| c14:0 | Myristic | 159 | 135 | 101 |
| c16:0 | Palmitic | 367 | 348 | 325 |
| c18:0 | Stearic | 81 | 89 | 98 |
| c18:1 | Vaccenic | 72 | 65 | 57 |
| c18:1 | Oleic | 289 | 320 | 378 |
| c18:2 | Linoleic | 288 | 402 | 622 |
| c18:3 | aLA | 43 | 54 | 78 |
| c20:4 | ARA | 51 | 110 | 137 |
| c20:5 | EPA | 378 | 319 | 236 |
| c22:6 | DHA | 254 | 214 | 159 |

TABLE 5
Phytosterol and cholesterol content of existing lipid emulsions (Intralipid,
Smoflipid, and Omegaven) compared to phytosterol content of constituent
oils and new lipid emulsion formulations. Existing lipid emulsion phytosterol
content was obtained from Llop-Talaveron, J et al. (2020). Phytosterolaemia
associated with parenteral nutrition administration in adult patients. British
Journal of Nutrition, 123(12), 1365-1372. doi: 10.1017/S0007114520000574.
Phytosterol content testing of oils used in new lipid emulsions was performed
by Eurofins Microbiology Laboratories, North Kingstown, RI.

| | Total | | | | | | | | | |
|---|---|---|---|---|---|---|---|---|---|---|
| | phytosterols | | β-Sitosterol | | Campesterol | | Stigmasterol | | Cholesterol | |
| | (μg/ml) | | (μg/ml) | | (μg/ml) | | (μg/ml) | | (μg/ml) | |
| Fat emulsion | Mean | SD | Mean | SD | Mean | SD | Mean | SD | Mean | SD |
| Intralipid * | 422.4 | 130.5 | 240.9 | 59.2 | 55.2 | 33.8 | 115.3 | 45.5 | 313.9 | 77.6 |
| Smoflipid * | 124.2 | 15.3 | 93 | 9.9 | 9.6 | 3.2 | 16.1 | 3.9 | 455.8 | 112.4 |
| Omegaven * | 0 | 0 | 0 | 0 | 0 | 0 | 0 | 0 | 416.2 | 112.4 |
| Low | 74.3 | | 44.0 | | 6.8 | | 5.5 | | 0.9 | |
| Phytosterol | | | | | | | | | | |
| Soy | | | | | | | | | | |
| Lipoid Fish Oil | 87.2 | | 1.8 | | 3.6 | | 0.5 | | 1558.8 | |
| Cargill ARA | 326.5 | | 36.6 | | 18.6 | | 2.6 | | 1.5 | |
| Fungal Oil | | | | | | | | | | |
| NLE A | 91.0 | | 10.1 | | 4.5 | | 1.4 | | 1247.2 | |
| NLE B | 101.9 | | 15.0 | | 5.5 | | 1.9 | | 1052.5 | |
| NLE C | 106.0 | | 22.2 | | 6.4 | | 2.7 | | 779.9 | |

Discussion: Three single-oil lipid emulsions were generated that pass USP <729> standards using fish oil, low phytosterol soybean oil, and arachidonic acid-rich fungal oil. These emulsions were then used to generate three new lipid emulsions for subsequent experiments that exceed recommended docosahexaenoic acid and arachidonic acid intake for premature infants.

### Experiment 2: Four-Week Administration of New Lipid Emulsions as Only Source of Fat in Juvenile C57Bl/6J Mice

Purpose: Evaluate whether the long-term (4 week) provision of new lipid emulsions as a sole fat source to juvenile mice affects plasma, liver, and brain fatty acid composition, to support their use in the neonatal population as a source of docosahexaenoic acid and arachidonic acid that prevents essentially fatty acid deficiency.

Methods: Four-week old male C57Bl/6J mice (Jackson Laboratory, Bar Harbor, ME) were obtained and randomized to either standard rodent chow (Prolab Isopro RMH 3000, LabDiet, St. Louis, MO) or an ad lib liquid fat-free high carbohydrate diet (20% dextrose, 2% amino acids, 30 mEq/L sodium, 20 mEq/L potassium, 15 mEq/L calcium, 10 mEq/L magnesium, 10 mMol/L phosphate, 36.67 mEq/L chloride, 19.4 mEq/L acetate, multivitamins, trace elements). Chow mice received daily orogastric gavage of normal saline (10 ml/kg), while the liquid fat-free diet mice received daily orogastric gavage of one of four lipid emulsions (10 ml/kg, 2 g/kg/day fat) as the sole source of fatty acids. After four weeks (at eight weeks old), mice were euthanized with CO₂; liver and brain tissue was flash frozen in liquid nitrogen and stored at −80° for further analysis. Liver and frontal cortex samples (n=5/group) were sent to an independent CLIA-certified laboratory for fatty acid analysis via gas chromatography with flame ionization detection (Omegaquant, Sioux Falls, SD).

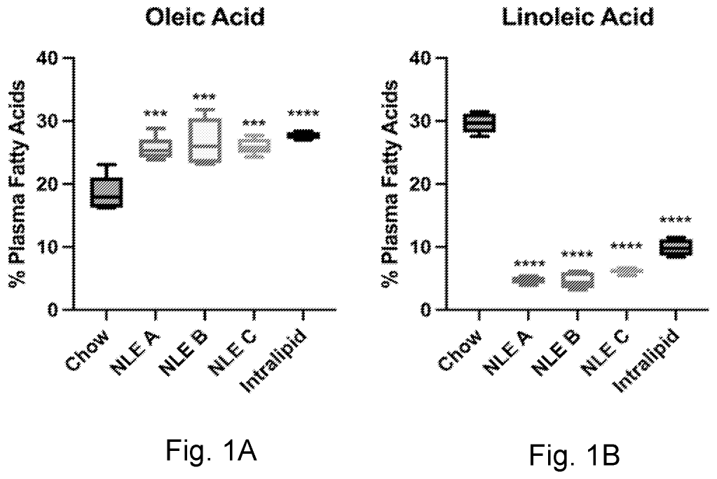

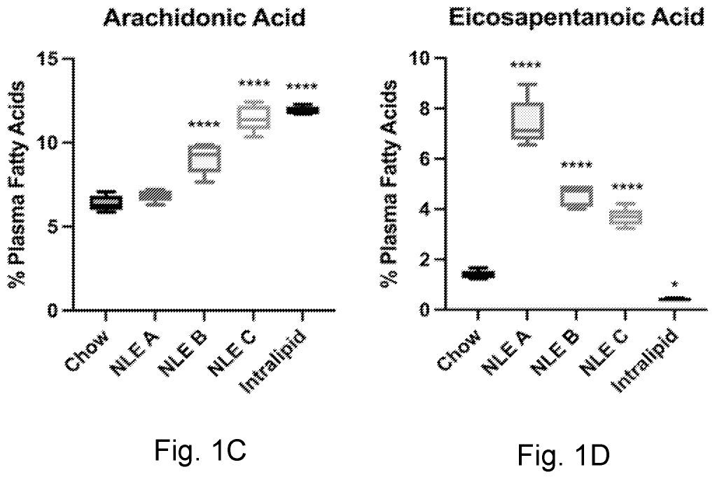

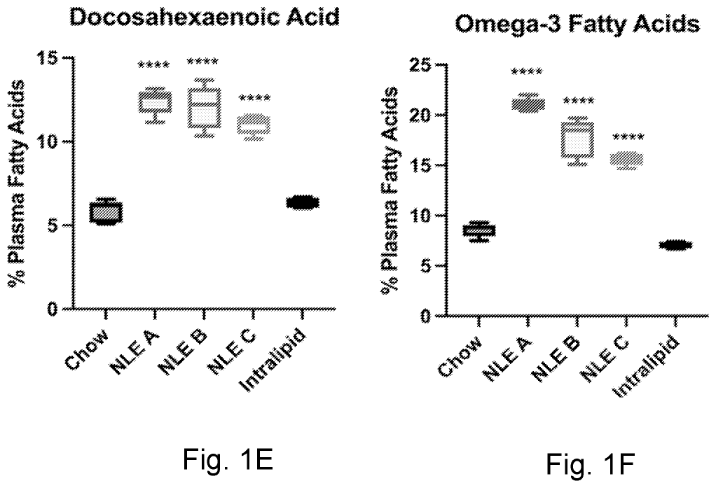

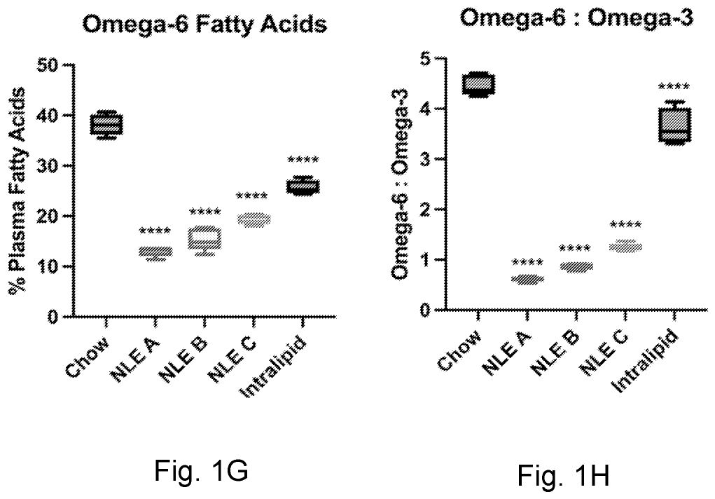

Results: The total fatty acid content in frontal cortex tissue was similar among groups (chow mean 32.7±1.0 μg/ml vs. Intralipid mean 34.2±0.4 μg/mg) while varying substantially in liver tissue (chow mean 27.3±2.2 μg/mg vs. Intralipid mean 70.1 μg/mg±22.0). Significant variability in the liver tissue fat content is attributable to greatly increased oleic acid content in the non-chow animals, particularly in the intralipid group. Thus, frontal cortex fatty acid composition was compared as a percentage of total cortex fatty acids, while liver fatty acid composition was compared as fatty acid content per unit liver tissue. FIGS. 1A-1H demonstrate the fatty acid composition of plasma, liver, and frontal cortex in the various groups.

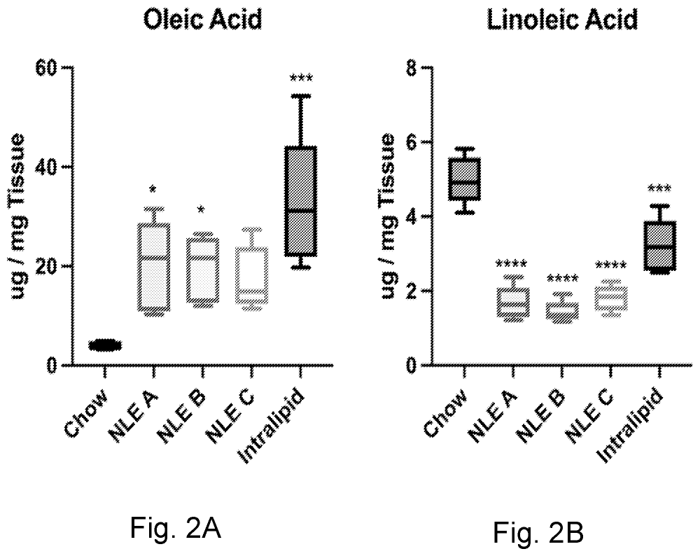

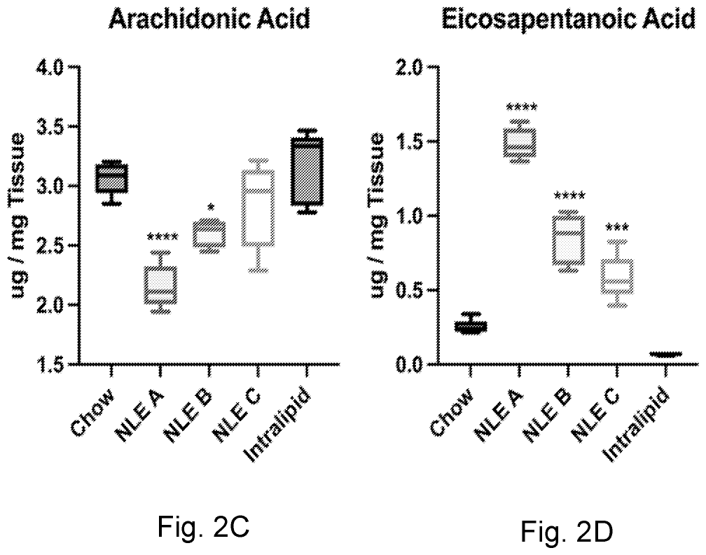

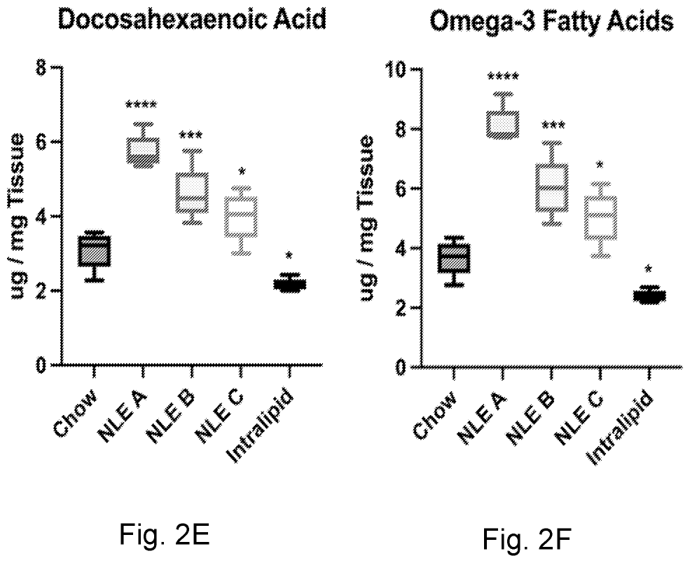

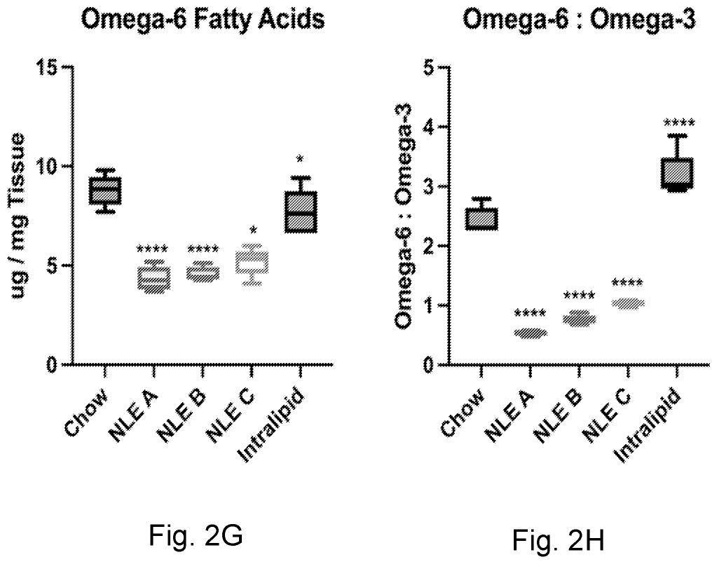

Mice in the NLE A, B, and C groups demonstrated elevated omega-3 fatty acids, decreased omega-6 fatty acids, and decreased omega-6:omega-3 ratio consistent with the provision of higher omega-3 fatty acids in plasma and liver tissue (FIGS. 2A-2H). In frontal cortex tissue, omega-3 fatty acids were similar between the chow control and the NLE A, NLE B, and NLE C groups. Intralipid demonstrated a pro-inflammatory fatty acid profile, with decreased omega-3 fatty acids and increased omega-6:omega-3 ratio in both liver and frontal cortex tissue compared to chow.

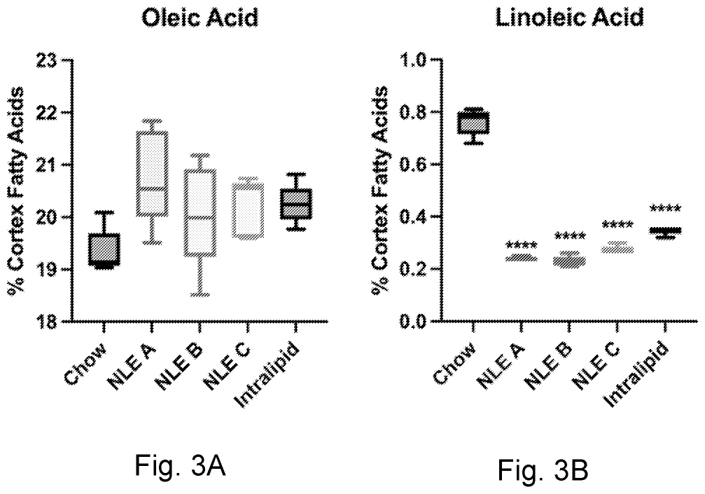

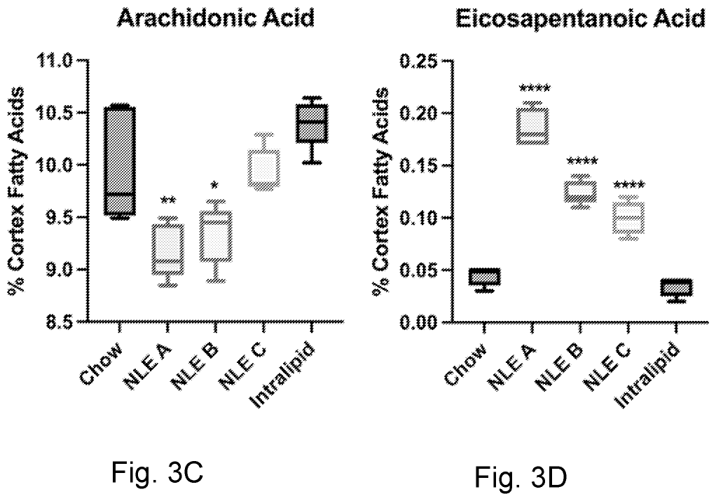

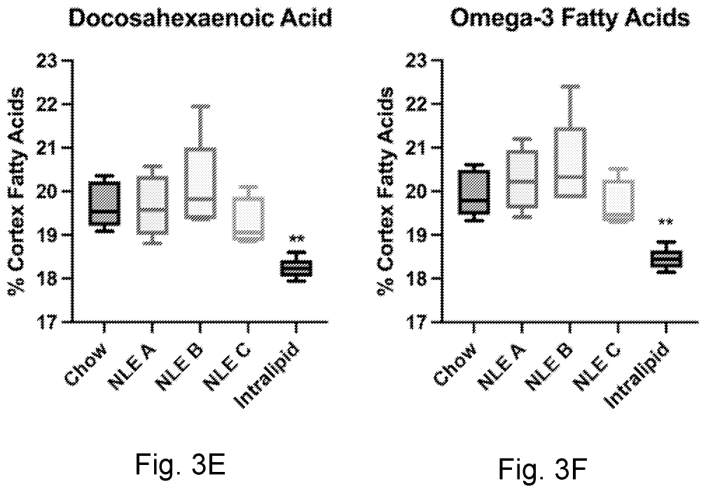

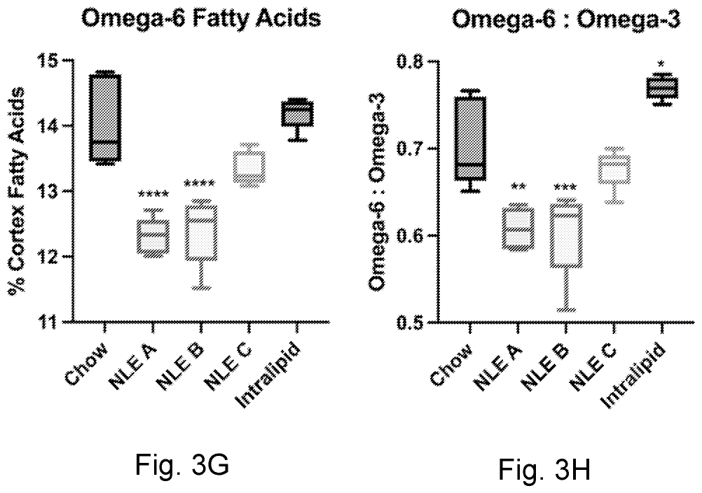

NLE A, B, and C mice all demonstrated elevated docosahexaenoic acid in plasma and liver tissue compared to chow and Intralipid, while maintaining similar docosahexaenoic acid in frontal cortex tissue compared to chow (FIGS. 3A-3H). Notably, Intralipid mice had substantially lower docosahexaenoic acid in liver and frontal cortex tissue than chow. NLE A, NLE B, and NLE C mice also had elevated eicosapentanoic acid in plasma, liver, and brain compared to chow and Intralipid mice. In plasma, mice in the NLE B, NLE C, and Intralipid groups have elevated arachidonic acid compared to chow, while NLE A is similar to chow. NLE A and NLE B have somewhat decreased arachidonic acid compared to chow in the liver and frontal cortex tissue.

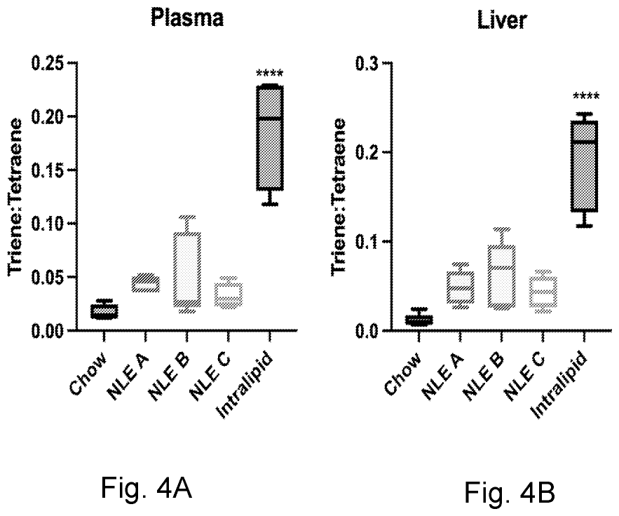

Triene: tetraene ratios were evaluated for prevention of biochemical essential fatty acid deficiency (≥0.2 is diagnostic for essential fatty acid deficiency). Notably, NLE A, B, and C all maintained normal triene: tetraene ratios in plasma and liver, which were similar to the chow control group, while the Intralipid group demonstrated significant elevations compared to chow control consistent with borderline essential fatty acid deficiency (FIGS. 4A-4B) (plasma 2/4 mice>0.2, mean 0.19±0.01, P<0.001; liver 3/5 mice>0.2, mean 0.19±0.05, P<0.001).

Conclusion: In juvenile mice provided long-term (4 week) daily lipid emulsion dosing as a sole fat source, NLE A, NLE B, and NLE C prevent essential fatty acid deficiency with similar triene: tetraene ratio to chow control, while Intralipid resulted in significantly elevated triene: tetraene ratio consistent with borderline essential fatty acid deficiency. Compared to chow control and Intralipid, NLE A, NLE B, and NLE C increase the omega-3 fatty acid content and decreases the omega-6:omega-3 ratio of plasma, liver, and frontal cortex, consistent with an anti-inflammatory fatty acid profile.

### Experiment 3: Administration of New Lipid Emulsions in a Murine Model of Parenteral Nutrition-Associated Liver Disease

Purpose: Evaluate whether the new lipid emulsions prevent biochemical liver injury and hepatosteatosis in a murine model of parenteral nutrition-induced liver disease.

Methods: Eight-week old male C57Bl/6J mice (Jackson Laboratory, Bar Harbor, ME) were obtained and randomized to either standard rodent chow (Prolab Isopro RMH 3000, LabDiet, St. Louis, MO) or an ad lib liquid fat-free high carbohydrate diet (20% dextrose, 2% amino acids, 30 mEq/L sodium, 20 mEq/L potassium, 15 mEq/L calcium, 10 mEq/L magnesium, 10 mMol/L phosphate, 36.67 mEq/L chloride, 19.4 mEq/L acetate, multivitamins, trace elements). Mice receiving the liquid fat-free high carbohydrate diet were administered one of five lipid emulsions or isovolumetric saline intravenously every other day via tail vein injection (4.8 g fat/kg body weight/dose), while chow mice received isovolumetric saline tail vein injections (Chow group). After 19 days, mice were euthanized with CO₂. Plasma was assessed for biochemical markers of liver injury using the Vetscan VS2 (Zoetis, Parsippany, NJ). Plasma fatty acid profiles were assessed as described previously (Carlson et al, 2019). Hematoxylin and eosin stained liver tissue was assessed by an independent board-certified veterinary pathologist using an established semi-quantitative scoring system for hepatic steatosis (Liang et al. Establishment of a General NAFLD Scoring System for Rodent Models and Comparison to Human Liver Pathology. PLoS ONE 9(12): e115922. doi: 10.1371/journal.pone.0115922). For normally distributed data, comparisons were made using one-way ANOVA tests and multiple comparisons between individual groups (against the HCD/saline group) made with Dunnett's multiple comparisons test. For non-normally distributed data, comparisons were made with the Kruskal-Wallis test and multiple comparisons between individual groups (against the HCD/saline group) made with Dunn's multiple comparisons test.

Results: Mice in all groups gained similar weight (P=0.12). Biochemical markers of livery injury are shown in FIGS. 5A-5C. Mice in the Omegaven, NLE A, NLE B, and NLE C groups all maintained a normal alanine aminotransferase level (and significantly lower than the saline control group), consistent with prevention of high carbohydrate diet-induced liver injury. Histologic evaluation of the livers was performed by an independent board-certified veterinary pathologist. Representative hematoxylin and eosin-stained liver sections are shown in FIG. 6. Mice receiving the high carbohydrate diet with NLE B or NLE C have no steatosis, while some mice receiving NLE A demonstrate mild micro- and macrovescular steatosis. Mice receiving a chow diet (with saline tail vein injection) have normal histology with no steatosis. All mice in the saline group had a lipidosis score of 2, which was significantly higher than the chow, NLE B, NLE C, and Omegaven groups which had no steatosis in any liver section (n=5/group, P=0.0051 for each comparison). There was no significant difference in lipidosis score between the saline group and the Intralipid group (2/5 liver sections had a lipidosis score of 3, P=0.61) or between the saline group and the NLE A group (2/5 liver sections had a lipidosis score of 2, P=0.27) (FIG. 7).

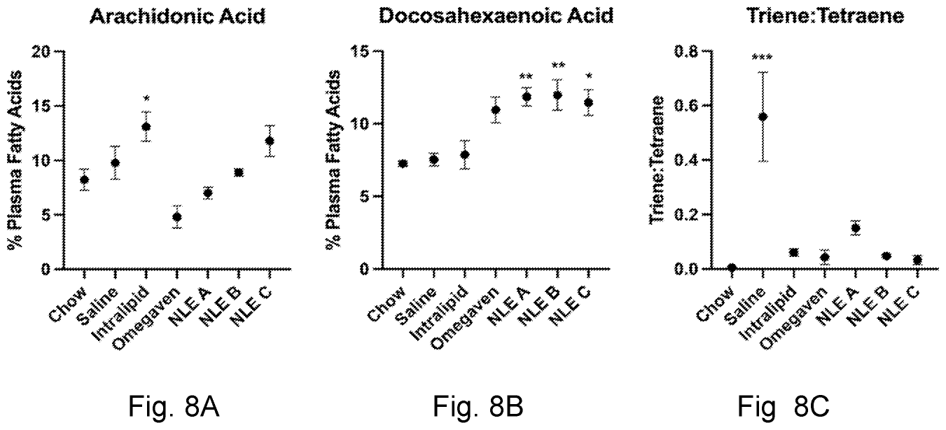

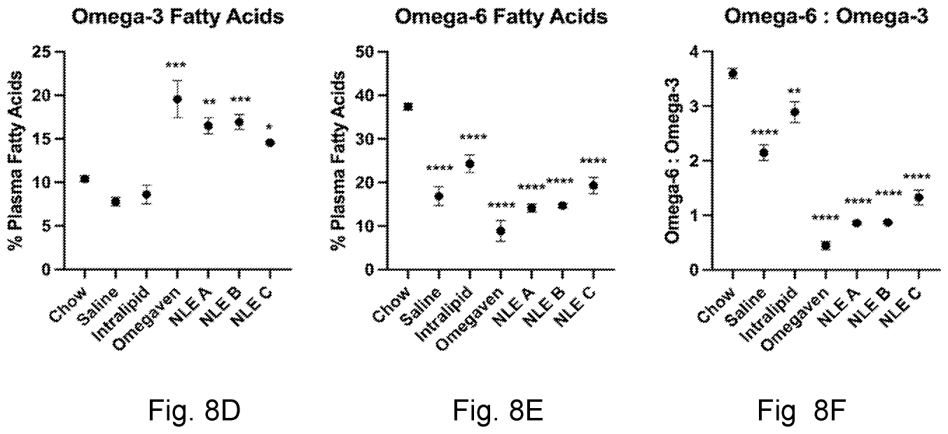

Compared to chow, mice receiving NLE A, NLE B, or NLE C demonstrated similar arachidonic acid, elevated docosahexaenoic acid, elevated omega-3 fatty acids, decreased omega-6 fatty acids, and decreased omega-6:omega-3 ratio (FIGS. 8A-8F). Mice receiving NLE A, NLE B, and NLE C also demonstrated similar (normal) triene:tetraene ratios to chow consistent with prevention of essential fatty acid deficiency. Mice in the saline group demonstrated markedly elevated triene: tetraene ratio consistent with essential fatty acid deficiency.

Conclusions: In a 19 day model of parenteral nutrition-induced liver injury and steatosis, administration of NLE A, NLE B, or NLE C via tail vein injection rescues mice from biochemical liver injury. Administration of NLE B or NLE C via tail vein injection prevents hepatic steatosis, with no steatosis noted in any sample. Administration of NLE A, NLE B, or NLE C prevented essential fatty acid deficiency with normal triene: tetraene ratios (FIGS. 8A-8F).

1. A composition comprising:
a) Arachidonic acid (ARA); and
b) Docosahexaenoic acid (DHA) and/or Eicosapentaenoic acid (EPA).

2. The composition of claim 1, comprising DHA.

3. The composition of claim 1, comprising EPA.

4. The composition of claim 1, comprising DHA and EPA.

5. (canceled)

6. The composition of claim 1, wherein the ratio of a) ARA to b) DHA and/or EPA is from 1:2 to 3:5 by weight.

7. (canceled)

8. The composition of claim 1, wherein the ratio of a) ARA to b) DHA and/or EPA is from 2:1 to 1:4 by weight.

9. (canceled)

10. The composition of claim 1, wherein the ratio of a) ARA to b) DHA and/or EPA is from 1:1 to 1:20 by weight.

11. (canceled)

12. The composition of claim 1, wherein the ratio of a) ARA to b) DHA and/or EPA is from 1:2 to 1:15 by weight.

13. (canceled)

14. The composition of claim 1, wherein the composition comprises further omega-6 fatty acids and/or omega-3 fatty acids.

15. The composition of claim 14, wherein the ARA and DHA and/or EPA comprise at least 10% of the total omega-3 fatty acids and omega-6 fatty acids.

16.-22. (canceled)

23. The composition of claim 1, wherein the ARA comprises at least 20% of the total omega-6 fatty acids.

24.-25. (canceled)

26. The composition of claim 1, wherein the DHA and/or EPA comprises at least 20% of the total omega-3 fatty acids.

27.-37. (canceled)

38. The composition of claim 1, wherein the ratio of omega-6 fatty acids to omega-3 fatty acids is from 8.41:1 to 1:4 by weight.

39.-55. (canceled)

56. The composition of claim 1, wherein the total triglyceride+diglyceride content of the DHA and/or EPA comprises no more than 10% diglyceride.

57.-62. (canceled)

63. The composition of claim 1, wherein the total triglyceride+diglyceride content of the ARA comprises no more than 10% diglyceride.

64.-68. (canceled)

69. The composition of claim 1, wherein the composition comprises no more than 5% (w/w) sterols.

70.-71. (canceled)

72. The composition of claim 1, wherein the composition comprises no more than 120 mg/L phytosterols.

73.-81. (canceled)

82. The composition of claim 1, wherein the composition comprises, or the ARA is provided in the form of, a plant or fungal oil;
wherein the composition comprises a fish oil, and/or the DHA and/or EPA is provided in the form of fish oil; and/or
wherein the composition comprises an algal oil, and/or the DHA and/or EPA is provided in the form of algal oil.

83.-115. (canceled)

116. A method of providing nutrition to a subject and/or promoting neurodevelopment in a subject, the method comprising administering the composition of claim 1 to the subject.

117. A method of treating premature birth, a neurological developmental deficit, retinal developmental deficit, bronchopulmonary developmental deficit, and/or bronchopulmonary dysplasia in a subject in need thereof, the method comprising administering the composition of claim 1 to the subject.

118.-172. (canceled)
